(19) United States
(12) Patent Application Publication
(10) Pub. No.: US 2019/0115105 A1
(43) Pub. Date: Apr. 18, 2019

(54) TANDEM DUPLICATOR PHENOTYPE (TDP) AS A DISTINCT GENOMIC CONFIGURATION IN CANCER AND USE THEREOF

(71) Applicant: The Jackson Laboratory, Bar Hor, ME (US)

(72) Inventors: Edison T. Liu, Bar HarBor, ME (US); Francesa Menghi, West Hartford, CT (US)

(73) Assignee: The Jackson Laboratory, Bar Hor, ME (US)

(21) Appl. No.: 16/087,467

(22) PCT Filed: Mar. 17, 2017
(86) PCT No.: PCT/US2017/022874

§ 371 Date: Sep. 21, 2018

Related U.S. Application Data
(60) Provisional application No. 62/312,802, filed on Mar. 24, 2016.

(51) Int. Cl. G16H 50/30; G16H 50/20; C12Q 1/6886; C12Q 1/6869; A61P 35/00; A61K 31/282

(52) U.S. Cl. CPC G16H 50/30; G16H 50/20; A61K 31/282; C12Q 1/6869; A61P 35/00; C12Q 1/6886

(57) ABSTRACT

The invention described herein provide a method to quantitatively determine the extent of tandem duplications (TDs) in certain cancer, and the use of the novel scoring metric to determine whether the cancers exhibiting tandem duplicator phenotype (TDP) and their increased susceptibility to platinum-based chemotherapeutic agents.

### REFERENCE TO RELATED APPLICATION

This application claims the benefit of the filing date of U.S. Provisional Application No. 62/312,802, filed on Mar. 24, 2016, the entire content of which is hereby incorporated by reference in their entirety.

### GOVERNMENT SUPPORT

This invention was made with government support under Grant No. P30CA034196 awarded by the National Cancer Institute, and Grant Nos. R21HG007554 and R21CA184851 awarded by the National Human Genome Research Institute and the National Cancer Institute, respectively. The U.S. Government has certain rights in this invention.

### BACKGROUND OF THE INVENTION

Cancer evolution is generally thought to result from the progressive accumulation of genomic lesions affecting key regulatory components of physiological cellular functions. Oncogenic changes can manifest as single nucleotide mutations, copy number alterations or variations (CNVs), such as deletions or duplications, and balanced rearrangements, including chromosomal translocations and inversions.

More recently, the systematic application of whole-genome sequencing (WGS) to the study of human cancer genomes, particularly the Next Generation Sequencing (NGS) studies, has uncovered more complex scenarios, where large portions of the genome are effected by a multitude of somatic structural variations, which either originate from a few unique catastrophic events (e.g., chromothripsis and chromoplexy) or result from the derangement of key molecular mechanisms leading to specific mutator phenotypes. These genome-wide structural variation patterns have been clearly associated with malignant phenotypes, although they are not known to be associated with a discernible driver mutation, and their potential to simultaneously de-regulate several oncogenic elements remains unclear.

Thus, despite their apparent relevance to the tumorigenic process, the causes of these genome-wide chromotypes, the cancer-driving oncogenic elements induced by these structural changes, and the clinical implications of these configurations remain unclear. Specifically, no favored therapeutic intervention has yet been discovered for any of these chromotypes. There is currently no clinical relevance for the presence of these genome-wide structural variation patterns.

Triple-negative breast cancer (TNBC) refers to any breast cancer that does not express the genes for estrogen receptor (ER), progesterone receptor (PR), and Her2/neu (ER⁻PR⁻HER2⁻). Most TNBC tumors exhibit aggressive behavior, distinct metastatic pattern, and very poor prognosis (compared to other breast cancer subtypes) following progression after standard chemotherapeutic regimens, regardless of the stage of disease at diagnosis (Cleere, Community Oncology 7(5): 203-211, 2010).

TNBC is more difficult to treat, and has limited treatment options, partly because no effective specific targeted therapy is readily available for TNBC, while the other breast cancer subtypes can benefit from targeted therapies directed against HER2 or ER. Thus TNBC treatment often requires combination therapies of surgery, radiotherapy, and/or chemotherapy.

The current standard of treatment for TNBC patients consists of cytotoxic therapies such as anthracyclines and taxanes (Cleere, 2010). A sequential combination of anthracycline, cyclophosphamide and taxane is the standard of care for moderate-to-high risk TNBC, with AC [doxorubicin, cyclophosphamide] followed by T [docetaxel], or simultaneous TAC [docetaxel, doxorubicin, cyclophosphamide] being the favored regimens.

Resistance to current standard therapies limits the available options for previously treated patients with metastatic TNBC to a small number of non-cross-resistant regimens such as cisplatin compounds, and there is currently no preferred standard chemotherapy for these patients (Andre and Zielinski, Annals of Oncology 23 (Supplement 6): vi46-vi51, 2012).

According to Cleere, recent clinical data for TNBC treatment reveal an important clinical feature of TNBC: patients' initial response to therapy is a crucial step in their treatment, and achieving a complete response is a major determining factor in their long-term survival. Thus, for better long term outcomes, TNBC, in particular, could benefit from using the most effective initial therapies capable of eradicating the disease (Cleere, 2010).

The importance of optimizing early-stage chemotherapy in TNBC is due to increased risk of recurrence within 3 years, increased risk of distant metastases and brain metastases with rapid progression from distant recurrence to death and lack of targets for therapy.

Also according to Cleere, despite recent advances in breast cancer treatment, relapse rates in TNBC remain high, with poor overall survival (OS). Such outcomes underscore the need for better up front treatment options for patients with this type of breast cancer (Cleere, 2010).

Several new treatment options are under investigation, including agents that target DNA damage and repair (e.g., platinum agents, PARP inhibitors, trabectedin (DNA-binding agent)), microtuble inhibition (e.g., ixabepilone), angiogenesis inhibition (e.g., Bevacizumab, sunitinib), EGFR targeting (e.g., Cetuximab, erlotinib), src targeting (e.g., Dasatinib), and mROR targeting (e.g., Temsirolimus, everolimus) (Cleere, 2010). For example, although some data suggests that platinum-based chemotherapy may increase response rates for early TNBC, further study is needed to better characterize the benefit of platinum agents in TNBC, including their effect on long-term survival and the extent of benefit in advanced TNBC (Cleere, 2010).

However, the specific adjuvant regimens that may be most effective for TNBC remain incompletely defined for both early stage and advanced disease (Wahba and Al-Hadaad, Cancer Biol Med 12:106-116, 2015). According to the same authors, TNBC is itself a heterogeneous group. Therefore, the identification of molecular biomarkers to predict response to specific chemotherapy is required to further improve treatment strategies with the current menu of chemotherapy options and future combinations with targeted therapies. This view is consistent with Andre and Zielinski (2012), which also call for improved treatment facilitated by biomarker-led understanding of subgroup molecular targets, which may predict benefit from currently approved agents, as well as newer targeted drugs.

In summary, current standard of treatment for TNBC patients consists of cytotoxic therapies such as anthracyclines and taxanes, while a few new treatment options are undergoing clinical investigation. However, relapse rates (especially early relapse within 3 years) in TNBC remain high, with poor OS, underscoring the importance and urgent unmet need to optimize early-stage chemotherapy in TNBC, with an emphasis on initial therapies capable of eradicating the disease.

### SUMMARY OF THE INVENTION

Promising data suggesting that platinum-based therapies (e.g., cisplatin and carboplatin) may be very effective in contrasting TNBC growth in the neoadjuvant (pre-operative) setting are being generated in ongoing clinical trials. However, they are still far from being considered standard of care options in the adjuvant setting.

Specific subgroups of patients with TNBC, such as BRCA1 mutation carriers, are more likely to benefit from platinum-base therapies, and tools to identify these better responsive patients are desirable for an optimal match between patient and treatment.

Data presented herein pertains to the study of a complex cancer genomic configuration, the tandem duplicator phenotype (TDP), which is characterized by the presence of a large number of somatic head-to-tail DNA segmental duplications (i.e., tandem duplications (TDs)) homogeneously distributed throughout the cancer genome. In a meta-analysis of over 3,000 cancer genomes, the most prevalent genetic features associated with this phenotype and those that may be responsible for its tumorigenic drive are identified. Furthermore, data presented herein shows an association between the extent of TDP and sensitivity to platinum-based chemotherapy in cell and primary xenograft models of triple negative breast cancer, thus providing a first indication of the utility of the TDP configuration as a predictive genomic biomarker in a clinical setting.

The present invention provides a method that comprises the steps of identifying or diagnosing a patient having a tumor that has TDP score greater than zero (i.e., positive TDP score). Such diagnosed patient candidate exhibits a greater sensitivity to a platinum-based therapeutic agent.

The present invention also provides a method of selecting a cancer patient as a candidate for treatment by a platinum-based therapeutic agent, comprising the step of identifying a cancer patient suffering from triple negative breast cancer, ovarian cancer, hepatocellular carcinoma, or endometrial carcinoma, wherein the identification of a cancer patient with a TDP score of greater than zero is predictive of increased responsiveness by the cancer patient for treatment by a platinum-based therapeutic agent.

Thus, in one aspect, the present invention provides a method of treating a cancer patient suffering from triple negative breast cancer, ovarian cancer, hepatocellular carcinoma, or endometrial carcinoma, the method comprising:
(a) obtaining a tumor sample from the cancer patient;
(b) determining tandem duplications of the tumor sample;
(c) determining a TDP Score using Formula (I):

$$TDP Score = TDP Raw Score + k = - \frac{{Σ}_{i} \vert {Obs}_{i} - {Exp}_{i} \vert}{TD} + k \qquad ( I )$$

wherein:
   TD is total number of tandem duplications,
   Obsᵢ is observed number of tandem duplications for each chromosome i,
   Expᵢ is expected number of tandem duplications for each chromosome i, and,
   k is 0.71; and
(d) administering a therapeutically effective amount of a platinum-based therapeutic agent to the patient, when the TDP Score is >0.

In certain embodiments, the determining step in step (b) is performed using whole-genome sequencing (WGS), SNP-array analysis, or both. For example, whole-genome sequencing (WGS) may be performed using Next Generation Sequencing (NGS), such as Next Generation Sequencing performed using Illumia HisSeq 2500 platform.

In certain embodiments, the total number of tandem duplications is mapped by breakpoint analysis.

In certain embodiments, the cancer is a triple negative breast cancer. The present invention discovered surprisingly that TNBC having a positive TDP score has high susceptibility against platinum-based therapeutic agents, particularly when such platinum-based therapeutic agents are used as front-line/primary treatment (as opposed to secondary treatment for, e.g., non-responsive or relapsed patients who have previously been treated by other non-platinum-based therapeutic agents). This provides a novel method of treatment for an unmet and long-felt need in this clinical crisis.

In certain embodiments, the cancer patient suffering from the triple negative breast cancer has not been treated previously with a chemotherapeutic agent.

In certain embodiments, the cancer patient suffering from the triple negative breast cancer has been treated with a chemotherapeutic agent other than a platinum-based therapeutic agent.

In certain embodiments, the cancer is an ovarian cancer. Preferably, the ovarian cancer is a serous ovarian cancer.

In certain embodiments, the cancer patient suffering from the ovarian cancer has not been treated previously with a chemotherapeutic agent.

In certain embodiments, the cancer patient suffering from the ovarian cancer has been treated with a chemotherapeutic agent other than platinum-based therapeutic agents.

In certain embodiments, the cancer is a hepatocellular carcinoma.

In certain embodiments, the cancer patient suffering from the hepatocellular carcinoma has not been treated previously with a chemotherapeutic agent.

In certain embodiments, the cancer patient suffering from the hepatocellular carcinoma has been treated with a chemotherapeutic agent other than platinum-based therapeutic agents.

In certain embodiments, the cancer is an endometrial carcinoma.

In certain embodiments, the cancer patient suffering from the endometrial carcinoma has not been treated previously with a chemotherapeutic agent.

In certain embodiments, the cancer patient suffering from the endometrial carcinoma has been treated with a chemotherapeutic agent other than platinum-based therapeutic agents.

In certain embodiments, the platinum-based therapeutic agent comprises cisplatin, carboplatin, oxaliplatin, nedaplatin, heptaplatin, lobaplatin, satraplatin, picoplatin, triplatin tetranitrate, phenanthriplatin, or a combination thereof.

In certain embodiments, the platinum-based therapeutic agent comprises cisplatin or carboplatin.

In certain embodiments, the platinum-based therapeutic agent is administered as a neoadjuvant (pre-operative) therapeutic agent.

In certain embodiments, the platinum-based therapeutic agent is administered as a adjuvant (post-operative) therapeutic agent.

Another aspect of the invention provides a method of identifying and selecting a cancer patient suffering from triple negative breast cancer, ovarian cancer, hepatocellular carcinoma, or endometrial carcinoma, as a candidate suitable for a platinum-based therapy, the method comprising:
(a) obtaining a tumor sample from the cancer patient;
(b) determining tandem duplications of the tumor sample;
(c) determining a TDP Score using Formula (I):

$$TDP Score = TDP Raw Score + k = - \frac{{Σ}_{i} \vert {Obs}_{i} - {Exp}_{i} \vert}{TD} + k \qquad ( I )$$

wherein:
   TD is total number of tandem duplications,
   Obsᵢ is observed number of tandem duplications for each chromosome i,
   Expᵢ is expected number of tandem duplications for each chromosome i, and,
   k is 0.71; and,
(d) identifying and selecting the patient as a candidate for the treatment of a platinum-based therapeutic agent, when the TDP Score is positive.

In certain embodiments, the method further comprises administering a therapeutically effective amount of the platinum-based therapy to the cancer patient.

In certain embodiments, the determining step in step (b) is performed using whole-genome sequencing (WGS), SNP-array analysis, or both. For example, the whole-genome sequencing (WGS) may be performed using Next Generation Sequencing (NGS), such as Next Generation Sequencing performed using Illumia HisSeq 2500 platform.

In certain embodiments, the cancer is a triple negative breast cancer.

In certain embodiments, the cancer is an ovarian cancer, such as a serous ovarian cancer.

In certain embodiments, the cancer is a hepatocellular carcinoma.

In certain embodiments, the cancer is an endometrial carcinoma.

In certain embodiments, the platinum-based therapeutic agent comprises cisplatin, carboplatin, oxaliplatin, nedaplatin, heptaplatin, lobaplatin, satraplatin, picoplatin, triplatin tetranitrate, phenanthriplatin, or a combination thereof.

In certain embodiments, the platinum-based therapeutic agent comprises cisplatin or carboplatin.

In certain embodiments, the platinum-based therapeutic agent is administered as a neoadjuvant (pre-operative) therapeutic agent.

In certain embodiments, the platinum-based therapeutic agent is administered as a adjuvant (post-operative) therapeutic agent.

Additional aspects and embodiments of the inventions are described in the paragraphs below:

In one aspect, the invention provides a method of treating a cancer in a patient having the cancer, comprising administering a therapeutically effective amount of platinum-based therapeutic agent to the patient, wherein the cancer is a TDP (tandem duplicator phenotype) cancer having a genomic configuration characterized by tandem duplications (TDs) evenly distributed across all chromosomes.

In certain embodiments, the cancer has a positive tandem duplicator phenotype score (TDP score) determined by Formula (I):

$$TDP Score = TDP Raw Score + k = - \frac{{Σ}_{i} \vert {Obs}_{i} - {Exp}_{i} \vert}{TD} + k \qquad ( I )$$

wherein:
   TD is total number of tandem duplications (e.g., tandem duplications mapped by breakpoint analysis),
   Obsᵢ is observed number of tandem duplications for each chromosome i,
   Expᵢ is expected number of tandem duplications for each chromosome i, and,
   k is a threshold value that normalizes the TDP Score for the TDP cancer to a positive value. For the purpose of this application, k is 0.71.

In certain embodiments, the distribution of the TDP Raw Score follows a trimodal pattern with a 1^{st}, a 2^{nd}, and a 3^{rd} mode, each mode independently having a peak and a standard deviation, and k equals the absolute value of the sum of the peak 2^{nd} mode TDP Raw Score and two standard deviations (SD) of the 2^{nd} mode.

In certain embodiments, the genomic configuration of the cancer is determined based on whole-genome sequencing (WGS) data, or SNP-array data (such as Affymetrix SNP 6.0 array data), or both.

In certain embodiments, the cancer is a triple negative breast cancer (TNBC), an ovarian cancer (e.g., a serous ovarian cancer), a hepatocellular carcinoma, or an endometrial carcinoma (e.g., a uterine corpus endometrial carcinoma (UCECs), or a cluster 4 endometrial carcinoma).

In certain embodiments, the cancer is not a prostate cancer, a glioblastoma, and a non-triple negative breast cancer (NTBC).

In certain embodiments, the cancer is characterized by: (i) genome-wide disruption of cancer genes; (ii) loss of cell cycle control and DNA damage repair; and/or (iii) increased sensitivity to cisplatin chemotherapy (in vitro and/or in vivo).

In certain embodiments, median span size of tandem duplications in the cancer is no more than about 1 Mb (or 1,000 kb), about 300 kb, about 200 kb, about 150 kb, about 100 kb, about 90 kb, about 50 kb, or about 10 kb.

In certain embodiments, span size of tandem duplications in the cancer is clustered around a size of about 10 kb, or about 250 kb, or both.

In certain embodiments, more than about 45%, 50%, 55%, 60%, 65%, 70%, 75%, 80%, or 85% (or 45-85%) of the tandem duplications in the cancer show overlapping microhomology between the two DNA segments contributing to the rearrangement junction.

In certain embodiments, more than about 45%, 50%, 55%, 60%, 65%, 70%, 75%, 80%, or 85% (or 45-85%) of the tandem duplications in the cancer utilize MH-mediated end joining (MMEJ) or microhomology-mediated break-induced replication as DNA repair mechanism to form the tandem duplications.

In certain embodiments, the tandem duplications in the cancer does not utilize nonallelic homologous repair (NAHR) as DNA repair mechanism to form the tandem duplications.

In certain embodiments, the cancer has a loss-of-function mutation in TP53, RAD51L1, WWOX, NF1, RB1, PTEN, and/or BRCAL

In certain embodiments, the cancer has a gain-of-function mutation in PAX8, ERBB2, ERBB3, TERC, STAT2, CDK2, MYC, and/or a DNA replication gene and/or a cell cycle gene (such as CCNE1, CDT1, MCM2, MCM6 and MCM10).

In certain embodiments, the platinum-based therapeutic agent comprises cisplatin, carboplatin, satraplatin, picoplatin, oxaliplatin, nedaplatin, lobaplatin, heptaplatin, Triplatin, LA-12, dicycloplatin, phosphaplatin, phenanthriplatin, any one or more of the above encapsulated within a macrocycle (such as cucurbit[n]urils, n-cyclodextrins and calix[n]arenes), a nanoparticle formulation thereof with a micelle/liposome (e.g., Aroplatin, SPI-77, LiPlaCis, Lipoplatin), polymer (e.g., ProLindac, and polyamidoamine (PAMAM) dendrimer bound platin), protein (e.g., transferrin-bound platin), metallic nanoparticle (e.g., gold or iron oxide nanoparticle-bound platin), or carbon nanotube scaffold, and/or an actively targeted platin thereof (such as platins tethered to a substrate or nutrient including vitamin, steroid, amino acid, and sugar; platins linked to an antibody or targeting peptide such as RGD sequence or TAT-fragment; and platins linked to a targeting aptamer).

In certain embodiments, the method further comprises administering to the patient a PARPi (Poly(ADP-ribose) polymerase inhibitor, such as CEP-6800).

In certain embodiments, the method further comprises selecting the patient for treatment based on the presence of the TDP cancer in the patient.

Another aspect of the invention provides a method of identifying a patient as a candidate for a platinum-based therapy for a cancer of the patient, the method comprising obtaining a TDP Score, based on Formula (I), of the cancer of the patient, and selecting the patient as the candidate for a platinum-based therapy if the TDP Score is positive, or is indicative of a genomic configuration characterized by tandem duplications (TDs) evenly distributed across all chromosomes.

In certain embodiments, the TDP Score is not indicative of a localized segmental amplifications with tandem duplications (TDs).

In certain embodiments, the distribution of the TDP Raw Score follows a trimodal pattern with a 1^{st}, a 2^{nd}, and a 3^{rd} mode, each mode independently having a peak and a standard deviation, and k equals the absolute value of the sum of the peak 2^{nd} mode TDP Raw Score and two standard deviations (SD) of the 2^{nd} mode.

In certain embodiments, the genomic configuration of the cancer is determined based on whole-genome sequencing (WGS) data, or SNP-array data (such as Affymetrix SNP 6.0 array data), or both.

In certain embodiments, the cancer is a triple negative breast cancer (TNBC), an ovarian cancer (e.g., a serous ovarian cancer), a hepatocellular carcinoma, or an endometrial carcinoma (e.g., a uterine corpus endometrial carcinoma (UCECs), or a cluster 4 endometrial carcinoma).

In certain embodiments, the cancer is not a prostate cancer, a glioblastoma, and a non-triple negative breast cancer (NTBC).

In certain embodiments, the platinum-based therapy comprises cisplatin, carboplatin, satraplatin, picoplatin, oxaliplatin, nedaplatin, lobaplatin, heptaplatin, Triplatin, LA-12, dicycloplatin, phosphaplatin, phenanthriplatin, any one or more of the above encapsulated within a macrocycle (such as cucurbit[n]urils, n-cyclodextrins and calix[n]arenes), a nanoparticle formulation thereof with a micelle/liposome (e.g., Aroplatin, SPI-77, LiPlaCis, Lipoplatin), polymer (e.g., ProLindac, and polyamidoamine (PAMAM) dendrimer bound platin), protein (e.g., transferrin-bound platin), metallic nanoparticle (e.g., gold or iron oxide nanoparticle-bound platin), or carbon nanotube scaffold, and/or an actively targeted platin thereof (such as platins tethered to a substrate or nutrient including vitamin, steroid, amino acid, and sugar; platins linked to an antibody or targeting peptide such as RGD sequence or TAT-fragment; and platins linked to a targeting aptamer).

In certain embodiments, the method further comprises administering to the patient the platinum-based therapy comprising cisplatin, carboplatin, satraplatin, picoplatin, oxaliplatin, nedaplatin, lobaplatin, heptaplatin, Triplatin, LA-12, dicycloplatin, phosphaplatin, phenanthriplatin, any one or more of the above encapsulated within a macrocycle (such as cucurbit[n]urils, n-cyclodextrins and calix[n]arenes), a nanoparticle formulation thereof with a micelle/liposome (e.g., Aroplatin, SPI-77, LiPlaCis, Lipoplatin), polymer (e.g., ProLindac, and polyamidoamine (PAMAM) dendrimer bound platin), protein (e.g., transferrin-bound platin), metallic nanoparticle (e.g., gold or iron oxide nanoparticle-bound platin), or carbon nanotube scaffold, and/or an actively targeted platin thereof (such as platins tethered to a substrate or nutrient including vitamin, steroid, amino acid, and sugar; platins linked to an antibody or targeting peptide such as RGD sequence or TAT-fragment; and platins linked to a targeting aptamer).

In certain embodiments, the method further comprises administering to the patient a PARPi (Poly(ADP-ribose) polymerase inhibitor, such as CEP-6800).

Another aspect of the invention provides a method of predicting the outcome of a cancer treatment in a patient having a cancer, wherein the cancer treatment comprises administering a therapeutically effective amount of a platinum-based therapeutic agent to the patient, the method comprising: determining a TDP Score, based on Formula (I), of the cancer, wherein a positive TDP Score is indicative of a favorable outcome, and a negative TDP Score is indicative of an unfavorable outcome.

In certain embodiments, the method further comprises administering, or continuing to administer, the therapeutically effective amount of the platinum-based therapeutic agent to the patient if the TDP Score is positive.

In certain embodiments, the method further comprises discontinuing further treatment of the patient with the platinum-based therapeutic agent if the TDP Score is negative.

In another aspect, the present invention provides a method of treating a cancer patient suffering from adrenocortical carcinoma, esophageal carcinoma, stomach adeno-carcinoma, lung squamous cell, or pancreatic adeno-carcinomas, the method comprising:
(a) obtaining a tumor sample from the cancer patient;
(b) determining tandem duplications of the tumor sample;
(c) determining a TDP Score using Formula (I):

$$TDP Score = TDP Raw Score + k = - \frac{{Σ}_{i} \vert {Obs}_{i} - {Exp}_{i} \vert}{TD} + k \qquad ( I )$$

wherein:
   TD is total number of tandem duplications,
   Obsᵢ is observed number of tandem duplications for each chromosome i,
   Expᵢ is expected number of tandem duplications for each chromosome i, and,
   k is 0.71; and
(d) administering a therapeutically effective amount of a platinum-based therapeutic agent to the patient, when the TDP Score is >0.

Another aspect of the invention provides a method of identifying and selecting a cancer patient suffering from adrenocortical carcinoma, esophageal carcinoma, stomach adenocarcinoma, lung squamous cell, or pancreatic adeno-carcinomas, as a candidate suitable for a platinum-based therapy, the method comprising:
(a) obtaining a tumor sample from the cancer patient;
(b) determining tandem duplications of the tumor sample;
(c) determining a TDP Score using Formula (I):

$$TDP Score = TDP Raw Score + k = - \frac{{Σ}_{i} \vert {Obs}_{i} - {Exp}_{i} \vert}{TD} + k \qquad ( I )$$

wherein:
   TD is total number of tandem duplications,
   Obsᵢ is observed number of tandem duplications for each chromosome i,
   Expᵢ is expected number of tandem duplications for each chromosome i, and,
   k is 0.71; and,
(d) identifying and selecting the patient as a candidate for the treatment of a platinum-based therapeutic agent, when the TDP Score is positive.

It should be understood that all embodiments described herein, including those described in the numbered paragraphs and those only in the examples, can be combined with any one or more other embodiment(s) unless explicitly disclaimed.

### BRIEF DESCRIPTION OF THE DRAWINGS

FIGS. 1A and 1B show Tandem duplicator phenotype (TDP) scoring and sample classification. FIG. 1A shows Circos plots showing structural variations of representative cancer genomes with different levels of TDP scores. For each plot, sample ID, TDP score, and number of tandem duplications over the total number of detected rearrangements are indicated (top to bottom). Structural variations were classified based on the four basic discordant paired-end mappings as tandem duplications (red), deletions (blue), unpaired inversions (green), or inter-chromosomal translocations (gray). FIG. 1B shows Trimodal distribution of the TDP score values across the 277 cancer samples examined.

FIGS. 2A-2D show genomic features of TDs in TDP and non-TDP tumors. FIG. 2A shows correlation of TDP score and median TD span size across the 277 tumor genomes analyzed by WGS. Horizontal lines indicate the overall median span size for the TDP and the non-TDP sample subgroups. A P-value was computed using the Student's t-test. FIG. 2B shows TD span distributions for the TDP and the non-TDP sample groups. TDP samples feature TDs with span peaks at ˜10 and ˜300 Kb. Non-TDP samples feature a much larger TD span range which homogeneously ranges from ˜1 to ˜10 Mb. A P-value for the distance between the two empirical distributions was generated using the Two-sample Kolmogorov-Smirnov test. FIG. 2C shows sequence analysis of TD breakpoints across TDP (n=4) and non-TDP (n=7) TNBC cell line genomes. OR and P-values were computed using the Fisher's exact test. FIG. 2D shows replication timing (RT) of genes located inside or on the boundary of TDs in TDP and non-TDP samples based on the breast cancer dataset. RT is expressed on a scale of 100 (early) to 1500 (late). P-values were computed based on the Mann-Whitney U test.

FIGS. 3A-3B show that the TDP is characterized by the coordinated perturbation of several cancer genes. FIG. 3A shows fold change in gene expression (breast tumor/normal breast) for genes frequently located inside or at the boundary of TDs in TDP tumors. (P-values by Mann-Whitney U Test). FIG. 3B shows that genes frequently affected by a TD breakpoint are enriched in anti-cancer genes (left), whereas genes frequently spanned by a TD are enriched in pro-cancer genes (middle). Short span TDs appear to most frequently interfere with anti-cancer as opposed to pro-cancer gene integrity (right). (P-values by Fisher's exact test).

FIGS. 4A-4G show that loss of the TP53 and BRCA1 tumor suppressor genes in the context of abnormal DNA replication may provide a permissive background for the insurgence of the TDP. FIG. 4A shows that TP53 mutation rate is recurrently higher in TDP compared to non-TDP samples. Odds ratio (OR) and corresponding P-values refer to the enrichment of TDP samples for samples with gene disruption. Percentages of TDP and non-TDP samples carrying the gene disruption are indicated in purple and green, respectively. FIGS. 4B-4C show that DNA-replication genes are consistently up-regulated in TDP vs. non-TDP samples. FIG. 4B shows top 10 GO terms significantly enriched in up-regulated genes (TDP vs. non-TDP) across the four different datasets analyzed. FIG. 4C shows heatmap of individual gene expression levels. Tumor samples are sorted based on tumor type and increasing TDP score. Only the 23 DEGs closely involved in DNA replication are shown. FIG. 4D shows that TDP samples are significantly enriched in BRCA1-low expressors across different tumor types. The threshold for low BRCA1 expression was defined based on the bimodal distribution of BRCA1 transcriptional levels in each individual dataset. Graph annotations are as in FIG. 4A. FIGS. 4E-4F show expression levels of the BRCA1 gene in TDP (purple) and non-TDP (green) triple negative breast cancer cell lines (FIG. 4E) and PDXs (FIG. 4F). TDP scores for these genomes were computed based on WGS data. BRCA1 somatic mutational status is indicated in brackets (mt, mutated; wt, wild type; na, not available). Pearson correlation coefficients (R) and their corresponding P-values are reported in each graph. Box-plots of BRCA1 expression values for TDP and non-TDP sample groups, log₂ fold changes and Student's t-test P-values are shown to the right. FIG. 4G shows that TOP samples are enriched for BRCA1-deficient tumors in both the TNB and OV datasets. BRCA1-loss is defined by the presence of germline or somatic mutations, or promoter methylation.

FIGS. 5A-5B show the TDP as a genomic marker for drug sensitivity. FIG. 5A shows that TDP scores correlate with cisplatin or carboplatin sensitivities in TNBC cell lines. Pearson correlation coefficients (R) and their corresponding P-values are reported in the graph. FIG. 5B shows that TDP scores associate with cisplatin sensitivity in vivo. Waterfall plots representing cisplatin response for eight TNB PDX models sorted by decreasing values of TDP scores. Response calls are indicated underneath each bar and were computed based on adapted RECIST criteria as described the examples.

FIGS. 6A-6C show structural variation (SV)-based score distributions and TDP status assignment. FIG. 6A shows trimodal distribution of TDP scores (n=266 samples with detected TDs) and cutoff for TDP classification. The trimodal distribution of TDP scores (top graph) were resolved using the normalmixEM function of the mixtools package in R. The fraction of samples belonging to each one of the three underlying normal distributions as well as the median and standard deviation (SD) values of each curve are shown in the table. The cutoff value to classify TDP samples is set to −0.71, which corresponds to the median+2×SDs of the second distribution. For better visualization, TDP scores were then centered around 0, as shown in FIG. 6C. FIG. 6B shows Scatter plot of TDP scores and TD numbers across tumor types (n=266 cancer genomes analyzed by WGS). A color code differentiates between tumor types that are TDP-enriched (red), TDP-depleted (blue) or with no significant TDP prevalence (grey), as indicated in Table 1. FIG. 6C shows distribution of the four basic structural variation scores across all cancer samples (n=277). A calculation analogous to the one used to compute TDP scores was applied to other structural variation types (deletion, inversion, and inter-chromosomal translocation). Only the distribution of TD scores (red) shows a clear sample sub-population characterized by distinctively higher scores.

FIGS. 7A-7F show TDP status prediction using array-based copy number data. TD-like segmental duplications were defined as copy number segments ranging between 1 Kb and 2 Mb in length, which showed an increase in copy number compared to both their neighboring segments (log₂ copy number (CN) ratio>=0.3), in a genomic region of otherwise homogenous copy number (difference in log₂ CN ratios between the two flanking segments<=0.3). FIGS. 7B-7C show Scatter plots of the number of TD (FIG. 7B) and TDP score (FIG. 7C) as predicted by whole-genome sequencing (WGS) or SNP-array copy number analysis for each one of the 81 TCGA cancer samples for which both types of data were available. (FIG. 7D) Sensitivity and specificity of TDP predictions based on copy number data. The TDP classification obtained based on WGS data is used as reference. (FIGS. 7E-7F) A more stringent differentiation between TDP and non-TDP samples improves the sensitivity (0.80) of TDP sample detection using SNP-array data, while maintaining a high degree of specificity (0.94). TDP tumors are defined as samples whose TDP score is higher than 0, as previously defined for WG-sequenced genomes. However, non-TDP samples are identified relative to a non-TDP SNP-array-based threshold computed based on the trimodal distribution of TDP scores across the entire SNP-array dataset (n=3,535 samples, threshold=−0.4).

FIGS. 8A-8G show molecular features of the genomic regions affected by TD breakpoints in TDP cancer genomes. (FIG. 8A) TD breakpoints cluster in gene-dense regions. Scatter plot showing a positive correlation between gene density and TD breakpoint density, computed per 10 Mb overlapping windows (1 Mb offset) along the entire genome. The combined TD coordinate data corresponding to the total of 50 TDP tumor genomes identified via WGS (including all available tumor types) were used in this analysis. Pearson correlation coefficient (R) and its corresponding P-value are reported in the graphs. (FIG. 8B) TDs are more likely to engage gene bodies than intergenic regions. Histogram bars represent the fraction of TD breakpoints which map within gene bodies in TDP genomes. A red line indicate the overall fraction of the genome occupied by gene bodies (including coding and non-coding sequences). ***, P-value<0.0001, computed using the binomial test. (FIG. 8C) Genes that are frequently located at the boundaries of TDs in TDP breast cancer genomes are generally expressed at high levels in the normal breast epithelium. Density plots represent the distribution of gene expression levels in normal breast tissue samples from the TCGA dataset (n=106). Median values for each distribution are indicated by dashed lines. A P-value (versus all RefSeq genes, n=20,502) was computed using the Mann-Whitney U Test. (FIG. 8D) Pol2 binding site enrichment in the proximity of breast cancer TD break points. Histogram bars correspond to the average odds ratio of 43 Pol2 ChlPseq data sets. ***, P<0.0001. (FIGS. 8E-8F) Histone modification mark enrichment/depletion in the proximity of breast cancer TD breakpoints. The results shown correspond to ChIP-seq datasets generated from the HMEC (FIG. 8E) and the vHMEC (FIG. 8F) cell lines. ***, P<0.0001. (FIG. 8G) Enrichment odds ratios for different histone modification marks in the proximity of breast cancer TD breakpoints in TDP breast tumors (n=23 tumors). ChIP-seq data for both the HMEC (top) and the vHMEC (bottom) cell lines are shown. Each bin on the horizontal axis represents a range of non-overlapping distances, e.g., a mark between 10 Kb and 20 Kb correspond to the enrichment in regions>10 Kb but<20 Kb from the nearest TD breakpoint.

FIGS. 9A-9E show that TD-like features specifically affect tumor suppressor genes and oncogenes. (FIG. 9A) Data from 418 TDP genomes assessed by SNP-array (TNB, NTNB, OV and UCEC datasets). P-values and odds ratios were computed using the Fisher's Exact test. (FIG. 9B) Histograms of frequencies for genes found at the boundaries (left) or inside (right) TD-like features in TDP tumors. Thresholds for frequency significance were defined based on 1,000 random gene sampling as described in Materials and Methods. Specific examples oncogenes (red) and tumor suppressor genes (blue) are indicated by arrows together with the number of unique TDP tumors in which they are affected. (FIG. 9C) Heatmap of co-occurrences for the top 25 genes found inside (red) and at the boundaries (blue) of TD-like features in TDP tumors. The top known cancer genes are indicated with the percentage of samples in which they are affected. (FIG. 9D) Co-occurrences are likely for genes that map within a short distance of each other and are therefore affected by the same TDs. The top 25 TD-inside genes shown in (FIG. 9C) are clustered based on chromosomal location. (FIG. 9E) Overview of all TD-like features at specific chromosomal loci. TD-like features are color-coded based on their effect on the gene of interest depicted in each graph (i.e., PAX8 (top) and PTEN (bottom)): gray, no effect; red, gene duplication (the target gene is located inside the TD); blue, gene disruption (the target gene located at the TD boundary, i.e., BP).

FIGS. 10A-10B show that short span TDs cause TSG disruption. (FIG. 10A) Short span TDs (<100 Kb) are more likely to fall completely within gene bodies than expected by chance. Short span TD genomic coordinates (n=3,086, based on WGS data from 50 TDP cancer genomes) were randomly permuted 1,000 times, preserving their sizes. At each permutation, the percentage of TDs integrally falling within gene bodies was recorded to generate the expected distribution. A red vertical line indicates the observed percentage of gene-embedded TDs, which exceeds all of the 1,000 permuted values. (FIG. 10B) UCSC Genome Browser screen shot showing the location of two short span TDs affecting the integrity of the PTEN tumor suppressor gene on Chr 10.

FIG. 11 shows TDP sample do not consistently show a higher mutation burden compared to non-TDP samples. Boxplots represent distributions in the number of unique genes per sample which are affected by non-silent somatic mutations. Although there is a significant increase in the overall number of mutations detected in TDP compared to non-TDP samples in the two breast cancer data sets analyzed and, with a more modest significant in the OV dataset, the trend was completely reversed in the UCEC dataset. TDP status was assigned based on SNP-array data. P-values were computed by using the Mann-Whitney U Test.

FIGS. 12A-12D show loss of BRCA1, but not of BRCA2, in TDP tumors. (FIG. 12A) Box plot of BRCA1 expression values for the TNB dataset. The BRCA1 gene is significantly down-regulated in TDP compared to non-TDP samples. Adj., adjusted. (FIG. 12B) Bimodal distribution of BRCA1 expression values was resolved to identify low-expressors. Low BRCA1 expressors are significantly enriched for TDP samples. (FIG. 12C) BRCA1 expression levels are inversely correlated with BRCA1 promoter methylation levels in the TNB and OV datasets (Pearson correlation: R=−0.61, P=2.3E-07 for the TNB dataset; R=−0.74, P<1.0E-05 for the OV dataset). The 10% most highly methylated samples at the BRCA1 promoter are indicated in red. (FIG. 12D) Contrary to the BRCA1 gene, the BRCA2 gene is more frequently mutated in non-TDP compared with TDP tumors across different tumor types. Only somatic mutations were analyzed for the UCEC dataset.

FIG. 13 shows that TDP-associated overexpression of DNA replication genes does not depend on their duplication status. Frequently up-regulated DNA replication genes that are also often affected by TDs across TDP samples were tested to assess whether their expression levels could be explained by the presence of TDs that increased their copy number status. For each gene, TDP samples with TDs spanning its entire length were removed from the analysis of differential gene expression. In all four cases, differences in expression levels between non-TDP and TDP tumors remained significant. ***, P<0.0001, Mann-Whitney U Test.

FIGS. 14A-14B show molecular and functional features discriminating between TDs found in TDP and non-TDP cancer genomes. (FIG. 14A) Graphic summary. (FIG. 14B) Oncoprints for the 90 TNBC samples for which RNAseq, SNP-array, and mutation data were available. BRCA1 down-regulation was defined in FIG. 12B. CCNE1 and CDT1 up-regulation was defined as a >2-fold increase in expression compared to the average gene expression level across all TNB non-TDP tumors. 13/33 TDP tumors show perturbation of three or four of the candidate genes, whereas only 2/57 non-TDP tumors do. Odds ratio=17.2, P-value=2.1E-05 (Fisher's exact test).

### DETAILED DESCRIPTION OF THE INVENTION

Described herein is a robust genomic metric able to identify cancers with a genomic configuration called tandem duplicator phenotype (TDP) characterized by frequent and distributed (e.g., relatively evenly distributed) tandem duplications (TDs). Enriched in certain triple negative breast, ovarian, endometrial, and liver cancers, among others, TDP tumors conjointly exhibit TP53-mutations, low expression of BRCA1, and increased expression of certain DNA replication genes pointing at re-replication in a defective checkpoint environment as a plausible causal mechanism. The resultant TDs in TDP augment global oncogene expression and disrupt tumor suppressor genes. Importantly, the TDP strongly correlates with platin-based chemotherapy (e.g., cisplatin) sensitivity, in both triple negative breast cancer cell lines and primary patient-derived xenografts (PDX). Thus, the TDP is a common cancer chromotype that coordinately alters oncogene/tumor suppressor expression with use as a marker for chemotherapeutic response.

Thus, in one aspect, the invention described herein provides a method of treating a cancer patient suffering from triple negative breast cancer, ovarian cancer, hepatocellular carcinoma, or endometrial carcinoma, the method comprising:
(a) obtaining a tumor sample from the cancer patient;
(b) determining tandem duplications of the tumor sample;
(c) determining a TDP Score using Formula (I):

$$TDP Score = TDP Raw Score + k = - \frac{{Σ}_{i} \vert {Obs}_{i} - {Exp}_{i} \vert}{TD} + k \qquad ( I )$$

wherein:
   TD is total number of tandem duplications (e.g., tandem duplications mapped by breakpoint analysis),
   Obsᵢ is observed number of tandem duplications for each chromosome i,
   Expᵢ is expected number of tandem duplications for each chromosome i, and,
   k is 0.71; and
(d) administering a therapeutically effective amount of a platinum-based therapeutic agent to the patient, when the TDP Score is >0.

As used herein, the “tumor sample” from the patient comprises, consists of, or consists essentially of diseased tissue from the cancer in the patient. The diseased tissue from the cancer may be from a primary cancer or a metastatic cancer.

As used herein, “determining tandem duplications” (of the tumor sample) refers to determining the total number of tandem duplications, as well as the distribution of the tandem duplications on each chromosome, in the genome of the tumor sample. For example, whole-genome sequencing (WGS) may provide a wealth of information about the point mutations and the various genomic aberrations in the cancer, including but not limited to deletion, insertion, inversion, and chromosomal translocations and/or rearrangements. The parameters that are useful for calculating the TDP Raw Score include the total number of tandem duplications in the genome, as well as the distribution of the tandem duplications on each chromosome.

As used herein, “therapeutically effective” means that the amount of platinum-based therapeutic agent administered that is sufficient to produce a clinical improvement in reducing cancer symptoms, such as a decrease in tumor cells, or a clinical sign, or an increase in feelings of well-being. It is contemplated that the dosing regimens for the compositions comprising a platinum-based therapeutic agent of the present invention are therapeutically effective.

In certain embodiments, the total number of tandem duplications is mapped by breakpoint analysis.

A related aspect of the invention provides a method of treating a cancer in a patient having the cancer, comprising administering a therapeutically effective amount of platinum-based therapeutic agent to the patient, wherein the cancer is a TDP (tandem duplicator phenotype) cancer having a genomic configuration characterized by tandem duplications (TDs) evenly distributed across all chromosomes. In certain embodiments, the cancer has a positive tandem duplicator phenotype score (TDP score) determined by Formula (I):

$$TDP Score = TDP Raw Score + k = - \frac{{Σ}_{i} \vert {Obs}_{i} - {Exp}_{i} \vert}{TD} + k \qquad ( I )$$

wherein:
TD is the total number of tandem duplications (e.g., tandem duplications mapped by breakpoint analysis),
Obsᵢ is observed number of tandem duplications for each chromosome i,
Expᵢ is expected number of tandem duplications for each chromosome i, and,
k is a threshold value that normalizes the TDP Score for the TDP cancer to a positive value. For the purpose of this application, k is 0.71.

$$TDP Raw Score = - \frac{{Σ}_{i} \vert {Obs}_{i} - {Exp}_{i} \vert}{TD} .$$

According to Formula (I),

Based on the total number of tandem duplications, each chromosome of the host genome under analysis, due to the known lengths of the respective chromosomes, will have an “expected” number of tandem duplications if the TDs are perfectly evenly distributed throughout the entire genome. Meanwhile, the observed number of actual TDs on each chromosome, obtained based on WGS, may or may not match the expected TD numbers.

In a cancer sample in which the TDs are perfectly evenly distributed throughout the entire genome, the Obsᵢ value and Expᵢ value are identical for each chromosome. Thus the TDP Raw Score is 0 (the maximum possible TDP Raw Score). On the other hand, for cancers with most TDs clustered on one or a few chromosomes, relatively large absolute values of Obsᵢ−Expᵢ are expected for each chromosome, resulting in a negative TDP Raw Score.

In certain embodiments, the distribution of the TDP Raw Score follows a trimodal pattern with a 1^{st}, a 2^{nd}, and a 3^{rd} mode, each mode independently having a peak and a standard deviation, and k equals the absolute value of the sum of the peak 2^{nd} mode TDP Raw Score and two standard deviations (SD) of the 2^{nd} mode.

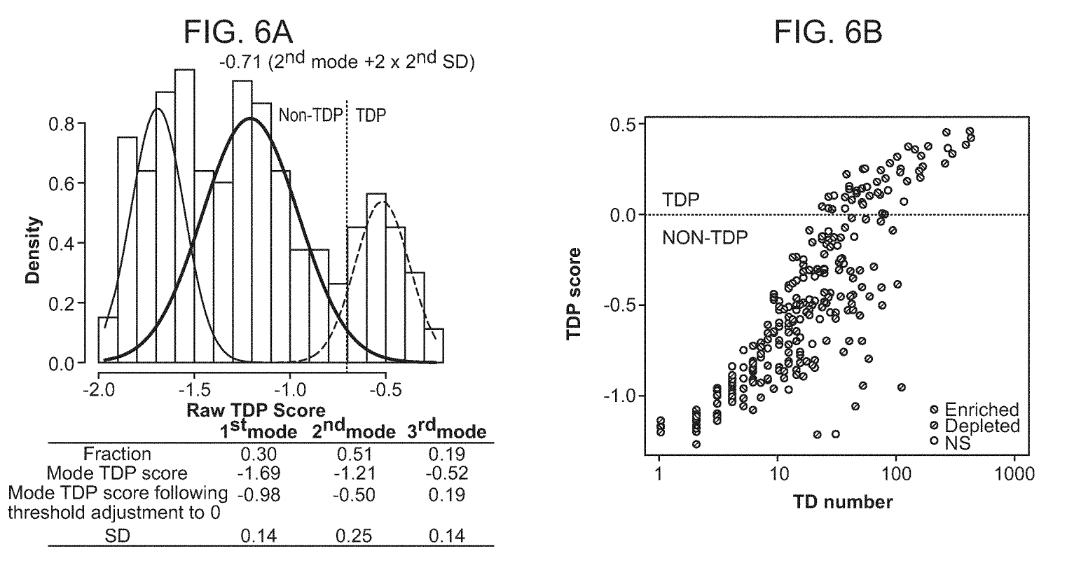

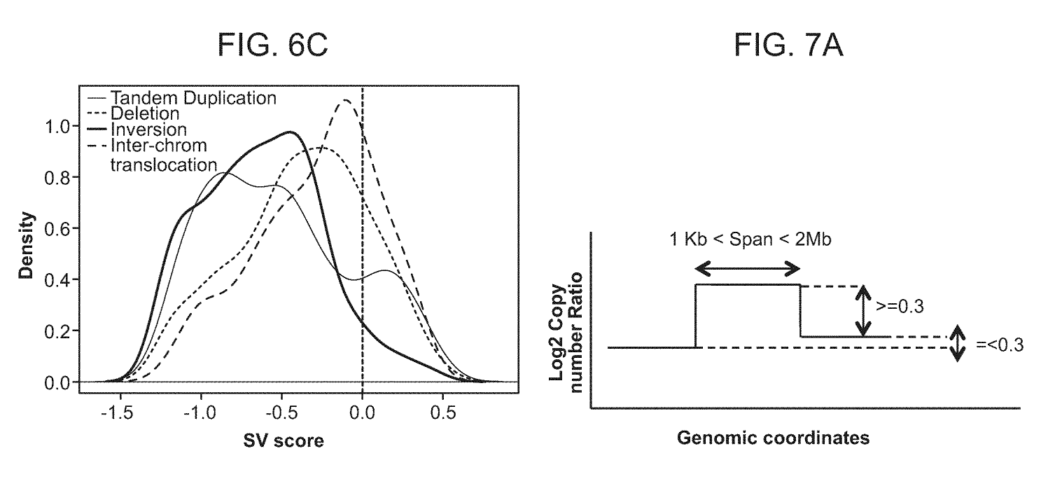

As illustrated in FIG. 6A, and partially explained in its legend, the trimodal distribution of TDP scores can be resolved using the normalmixEM function of the mixtools package in R, or an equivalent software. The fraction of samples belonging to each one of the three underlying normal distributions, as well as the median and standard deviation (SD) values of each curve can also be determined. In the data shown in FIG. 6A, the 1^{st} mode of TDP Raw Score distribution has a peak value of −1.69, and a SD of 0.14; the 2^{nd} mode of TDP Raw Score distribution has a peak value of −1.21, and a SD of 0.25; and the 3^{rd} mode of TDP Raw Score distribution has a peak value of −0.52, and a SD of 0.14. Thus the sum of the peak 2^{nd} mode TDP Raw Score (−1.21) and two standard deviations (SD) of the 2^{nd} mode (2×0.25) is −1.21+0.50=−0.71, which absolute value is 0.71 (k). Thus in certain embodiments, k is about 0.71.

With the above k value, the re-centered TDP Score, based on the TDP Raw Score and k value, is 0 or above for all TDP cancers in FIG. 6A, and a negative value for all non-TDP cancers in FIG. 6A.

In certain embodiments, the genomic configuration of the cancer, such as tandem duplications of the tumor sample, is determined based on whole-genome sequencing (WGS) data.

Whole-genome sequencing (WGS) is the process of determining the complete DNA sequence of an organism's genome at a single time. In this application, WGS is used to determine the genomic structures, including the presence of TD, inversion, deletion, etc., in a tumor cell.

Many high-throughput sequencing technologies (e.g., next-generation sequencing technologies) (NGS) are available for WGS. In certain embodiments, whole-genome sequencing can be conveniently performed by the Illumia HiSeq 2500 high throughput-sequencing instrument of Illumina.

Other suitable NGS methods include (but not limited to): massively parallel signature sequencing (MPSS), polony sequencing, 454 pyrosequencing, SOLiD sequencing, ion torrent semiconductor sequencing, DNA nanoball sequencing, heliscope single molecule sequencing, single molecule real time (SMRT) sequencing, nanopore DNA sequencing, tunnelling currents DNA sequencing, sequencing by hybridization, sequencing with mass spectrometry, microfluidic Sanger sequencing, microscopy-based techniques, RNAP sequencing, and in vitro virus high-throughput sequencing.

In certain embodiments, the genomic configuration of the cancer, such as tandem duplications of the tumor sample, is determined based on single nucleotide polymorphism (SNP)-array data (such as Affymetrix SNP 6.0 array data), or both WGS data and SNP-array data.

SNP (single nucleotide polymorphism), a variation at a single site in DNA, is the most frequent type of variation in the genome (e.g., about 85 million SNPs have been identified in the human genome). SNP array is a type of DNA microarray used to detect polymorphisms and copy number changes within a population.

The basic principles of SNP array are the same as the DNA microarray. These are the convergence of DNA hybridization, fluorescence microscopy, and solid surface DNA capture. The three mandatory components of the SNP arrays are: an array containing immobilized allele-specific oligonucleotide (ASO) probes; fragmented nucleic acid sequences of target, labelled with fluorescent dyes; and a detection system that records and interprets the hybridization signal.

The ASO probes are often chosen based on sequencing of a representative panel of individuals: positions found to vary in the panel at a specified frequency are used as the basis for probes. SNP chips are generally described by the number of SNP positions they assay. Two probes must be used for each SNP position to detect both alleles; if only one probe were used, experimental failure would be indistinguishable from homozygosity of the non-probed allele.

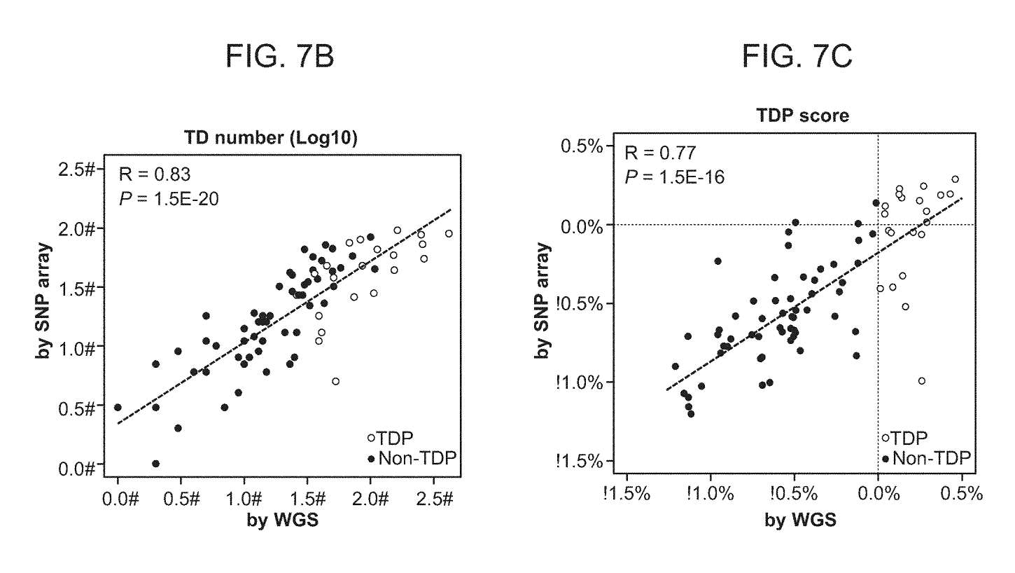

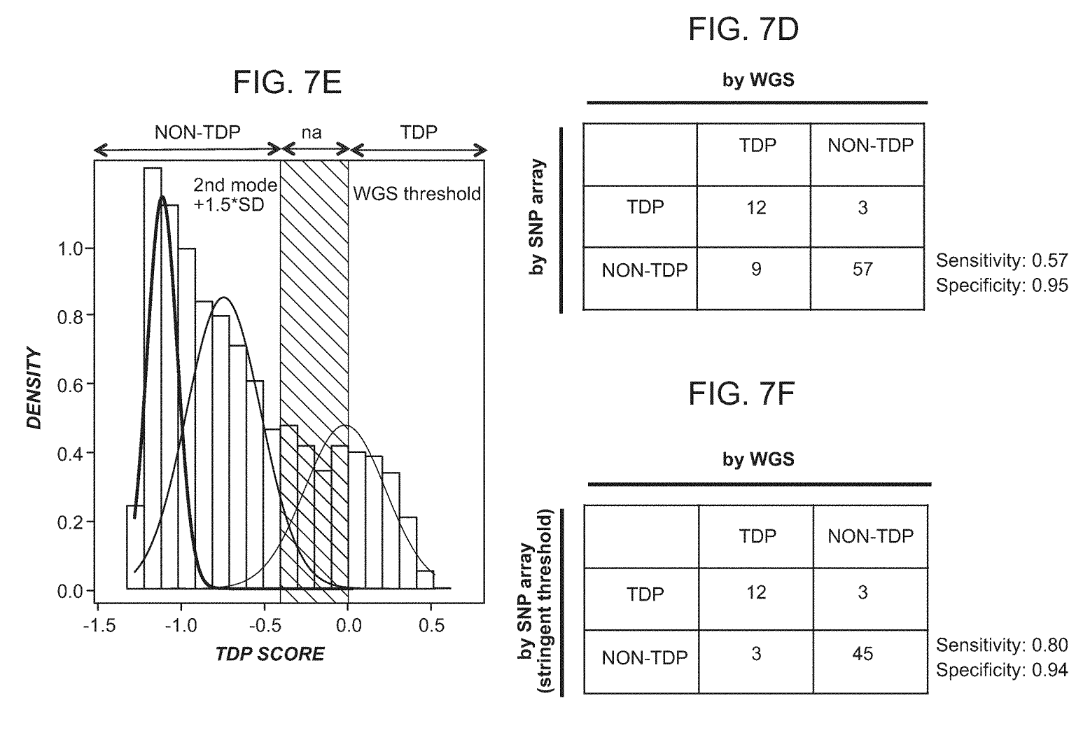

For example, as illustrated in FIG. 7A, TD-like segmental duplications can be defined as copy number segments ranging between 1 Kb and 2 Mb in length, which showed an increase in copy number compared to both their neighboring segments (e.g., log₂ copy number (CN) ratio>=0.3), in a genomic region of otherwise homogenous copy number (e.g., difference in log₂ CN ratios between the two flanking segments<=0.3). Once tandem duplications are so identified, TDP score can be calculated in substantially the same way as it is using WGS data according to Formula 1.

In certain embodiments, when SNP-array data is used, a more stringent differentiation between TDP and non-TDP samples may be used to improve the sensitivity of TDP sample detection using SNP-array data, while maintaining a high degree of specificity. See FIG. 7E. In this embodiment, TDP tumors are defined as samples whose TDP Score is higher than 0, as previously defined for WG-sequenced genomes. However, non-TDP samples are identified relative to a non-TDP SNP-array-based threshold computed based on the trimodal distribution of TDP scores across the entire SNP-array dataset (e.g., in FIG. 7E, n=3,535 samples, threshold=−0.4, which is arrived by adding 1.5 fold of SD for the 2^{nd} mode to the peak 2^{nd} mode TDP Score).

In certain embodiments, the cancer is a triple negative breast cancer (TNBC), an ovarian cancer (e.g., a serous ovarian cancer), a hepatocellular carcinoma, or an endometrial carcinoma (e.g., a uterine corpus endometrial carcinoma (UCECs), or a cluster 4 endometrial carcinoma). In certain embodiments, the cancer is not a prostate cancer. In certain embodiments, the cancer is not a glioblastoma. In certain embodiments, the cancer is not a non-triple negative breast cancer (NTBC).

In certain embodiments, the cancer is an adrenocortical carcinoma, esophageal carcinoma, stomach adeno-carcinoma, lung squamous cell, or pancreatic adeno-carcinomas.

In certain embodiments, the cancer is characterized by: (i) genome-wide disruption of cancer genes; (ii) loss of cell cycle control and DNA damage repair; and/or (iii) increased sensitivity to cisplatin chemotherapy (in vitro and/or in vivo).

In certain embodiments, median span size of tandem duplications in the cancer is no more than about 1 Mb (or 1,000 kb), about 500 kb, about 400 kb, about 300 kb, about 200 kb, about 150 kb, about 100 kb, about 90 kb, about 50 kb, or about 10 kb.

In certain embodiments, span size of tandem duplications in the cancer is clustered around a size of about 10 kb, or about 250 kb, or both.

In certain embodiments, the distribution of span size of tandem duplications in the (TDP) cancer genomes has two peaks, at about 10 kb and about 250 kb.

In certain embodiments, more than about 45%, 50%, 55%, 60%, 65%, 70%, 75%, 80%, or 85% (e.g., about 45-85%, about 50-80%, about 55-75%, about 60-75%, about 70-75%, or about 72%) of the tandem duplications in the cancer show overlapping microhomology between the two DNA segments contributing to the rearrangement junction.

In certain embodiments, more than about 45%, 50%, 55%, 60%, 65%, 70%, 75%, 80%, or 85% (e.g., about 45-85%, about 50-80%, about 55-75%, about 60-75%, about 70-75%, or about 72%) of the tandem duplications in the cancer utilize MH-mediated end joining (MMEJ) or microhomology-mediated break-induced replication as DNA repair mechanism to form the tandem duplications.

In certain embodiments, the tandem duplications in the cancer does not utilize nonallelic homologous repair (NAHR) as DNA repair mechanism to form the tandem duplications.

In certain embodiments, the cancer has a loss-of-function mutation in TP53, RAD51L1, WWOX, NF1, RB1, PTEN, and/or BRCAL

In certain embodiments, the cancer has a gain-of-function mutation in PAX8, ERBB2, ERBB3, TERC, STAT2, CDK2, MYC, and/or a DNA replication gene and/or a cell cycle gene (such as CCNE1, CDT1, MCM2, MCM6 and MCM10).

In certain embodiments, the platinum-based therapeutic agent comprises cisplatin, carboplatin, satraplatin, picoplatin, oxaliplatin, nedaplatin, lobaplatin, heptaplatin, Triplatin, LA-12, dicycloplatin, phosphaplatin, and/or phenanthriplatin.

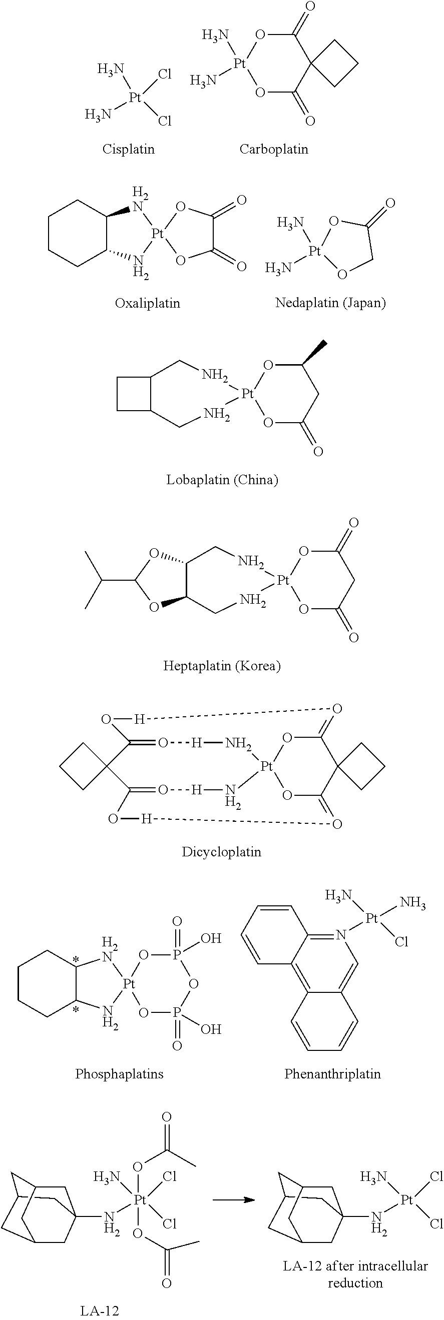

In certain embodiments, the platinum-based therapeutic agent comprises cisplatin, carboplatin, oxaliplatin, nedaplatin, heptaplatin, lobaplatin, satraplatin, picoplatin, triplatin tetranitrate, phenanthriplatin, or a combination thereof.

In certain embodiments, the platinum-based therapeutic agent comprises cisplatin.

In certain embodiments, the platinum-based therapeutic agent comprises carboplatin.

The problem of protein binding and peptide mediated degradation can in part be solved through the encapsulation of the platin drugs within macrocycles. A macrocycle is a short polymer that has ring-closed during synthesis to form a single loop structure. Macrocycles usually contain a hydrophobic cavity that can be accessed through one or more portals. The encapsulation of drugs in the cavity is controlled through hydrophobic effects as well as hydrogen bonds and/or ion-dipole bonds at the portals.

There are three main families of macrocycles relevant to the delivery of platin-based drugs. These include cucurbit[n]urils, n-cyclodextrins and calix[n]arenes, where n indicates the number of subunits that make up the macrocycles.

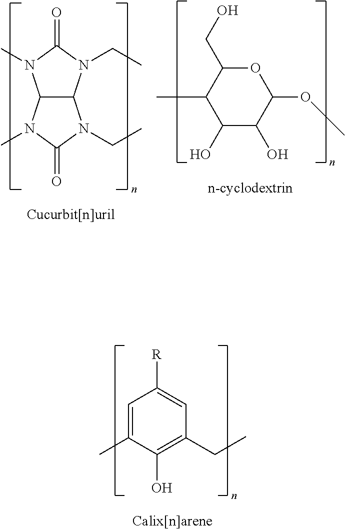

Thus in certain embodiments, the platinum-based therapeutic agent comprises any one or more of the above (cisplatin, carboplatin, satraplatin, picoplatin, oxaliplatin, nedaplatin, lobaplatin, heptaplatin, Triplatin, LA-12, dicycloplatin, phosphaplatin, and/or phenanthriplatin) encapsulated within a macrocycle. The macrocycle may be cucurbit[n]urils (e.g., n is 6, 7, or 8), n-cyclodextrins (e.g., n is 6, 7, or 8), or calix[n]arenes (e.g., n=4 for p-sulfonatocalixarene). For calix[n]arenes, R can be a variety of groups, preferably an anionic SO₃ group.

Cucurbit[n]urils, abbreviated as CB[n], are made by reacting glycoluril with formaldehyde in concentrated acid solutions. The product is a mixture of different sized cucurbit[n]urils with between 5 and 14 subunits. While not highly soluble in pure water, they become soluble upon forming host-guest complexes with drugs and in solutions with high salt concentrations, such as blood serum, and gastric and nasal fluids. Cucurbit[n]urils are relatively non-toxic, and can be formulated into oral tablets, topical creams and eye drop solutions. For drug delivery the homologues of six, seven and eight subunits are of most importance as these have a cavity that is ideally sized to store and release platins. At least 15 platin-based compounds have been examined with cucurbit[n]urils, including the cisplatin, oxaliplatin and triplatin.

In certain embodiments, cisplatin is complexed with cucurbit[n]urils, such as CB[7]. Cisplatin binds into the cavity of CB[7] so that the chloride ligands project into the cavity of the macrocycle. Such binding is stabilized by multiple hydrogen-bonds from the drug's ammine hydrogens to the CB[7]'s oxygens at its portals, thus greatly reducing cisplatin's rate of reaction with proteins and peptides and in vivo makes the drug statistically more effective in treating tumor xenografts, including xenografts resistant to cisplatin.

In certain embodiments, oxaliplatin is complexed with cucurbit[n]urils, such as CB[7]. Oxaliplatin forms host-guest complexes with CB[7] in such a way that the hydrophobic diaminocyclohexane ring is located within the macrocycle with the labile oxalate ligand protruding from one portal. The result is a much more stable formulation of the drug in both the solid and solution states, and which is 15-fold less reactive with methionine compared with normal oxaliplatin.

In certain embodiments, triplatin is complexed with cucurbit[n]urils, such as CB[7]. Triplatin has three platinum atoms joined via two diaminoalkane ligands. When encapsulated by cucurbit[n]urils, they form a 2:1 CB[n]-to-drug host-guest complex, where the macrocycles are located over the bridging ligands. In certain embodiments, the cytotoxicity and toxicity of triplatin is tuned by varying the size of the cucurbit[n]uril used. Similar to cisplatin and oxaliplatin, encapsulation of triplatin and other multinuclear platins by cucurbit[n]urils significantly reduces their reactivity, particularly with thiol-containing peptides.

Cyclodextrins are a family of oligosaccharide macrocycles approved for use in pharmaceutical dosage formulations. Examples of medicines that include n-cyclodextrins as delivery vehicles include Bridion, Zeldox IM and Movectro. In certain embodiments, cyclodextrins come in sizes of up to ten subunits, preferably six, seven or eight subunits, designated α-, β- and γ-cyclodextrin, respectively. All are soluble in water, although β-cyclodextrin is nephrotoxic and is not used in i.v. formulations. Every n-cyclodextrin contains a central cavity that is accessible through two portals.

Unlike cucurbit[n]urils, the portals of cyclodextrins are not symmetrical; they contain one major portal and one minor portal. One favorable characteristic of n-cyclodextrins, is the ease with which they can be functionalized. Groups to change their lipophilicity/hydrophilicity, or cancer targeting groups, are easily attached using standard chemical techniques. The partial encapsulation of three different platin agents by a carboxylated form of β-cyclodextrin slowed their degradation by glutathione by at least threefold.

Calix[n]arenes are a family of truncated bowl shaped macrocycles of para-substituted phenol monomers linked by methylene bridges. The hydrophobic cavity is accessible through only one portal as the bottom of the macrocycle is closed off by extensive hydrogen bonding between the phenol hydroxide groups. Native calix[n] arenes are soluble only in organic solvents with only one water soluble derivative known; p-sulfonatocalix[n]arenes, where n=4-8. An additional benefit of the negative charges of the sulfate groups, beyond making the macrocycle water soluble, is that they help to form host-guest complexes with positively charged drugs. In certain embodiments, the calix[n]arene is p-sulfonatocalix[n]arene, which is relatively non-toxic and have shown considerable potential in drug delivery.

The nature of the host-guest complexes formed with calix[n]arenes are dependent of the type of platin agent used. For mononuclear complexes a unique 2:2 complex is formed, where two platin molecules stack on top of each other and where each of their ends are covered by a calixarene molecule. The result is a supramolecular complex that resembles a molecular medicine capsule that is highly efficient at preventing drug degradation by glutathione. In contrast, dinuclear platinum agents form 1:1 host-guest complexes where the bridging ligand is located within the cavity of the calixarene and the platinum groups are located at the portal where they form iondipole and hydrogen bonds with the sulfate groups. In this configuration, the calix[n]arene provides no steric protection for dinuclear platins from glutathione attack and, as such, is not as useful for slowing drug degradation. Thus in certain embodiments, Calix[n]arenes are used for mononuclear platin drugs, like oxaliplatin.

In certain embodiments, to overcome poor selectivity for cancerous tissue compared with normal tissue, nanoparticle formulations are developed to better target cancerous tissue due to the enhanced permeability and retention (EPR) effect. The EPR effect is a function of the rapid growth of solid cancers where they develop large gaps between the endothelial cells, which trap and retain nanoparticles; these gaps are not present in normal tissue. There are a variety of scaffolds that can be utilized as nanoparticle delivery vehicles for platins, including micelles and liposomes, some polymers, metallic nanoparticles and carbon nanotubes.

Thus in certain embodiments, the platinum-based therapeutic agent is a nanoparticle formulation with a micelle or liposome scaffold (e.g., Aroplatin, SPI-77, LiPlaCis, Lipoplatin). One of the most successful methods of nanoparticle formulation for chemotherapeutic drug delivery are micelles and liposomes. Examples of successful drugs using liposomal formulations are doxorubicin (Doxil) and vincristine (VincaXome). Aroplatin and SPI-77 are liposomal formulations of platin drugs that underwent clinical trials. LiPlaCis is a liposomal formulation of cisplatin encapsulated in pro-anticancer ether lipids. This liposome is designed to release cisplatin inside cancer cells upon its degradation by the secretory phospholipase A2 (sPLA2) enzyme, which is overexpressed in many different types of cancers and therefore provides some specificity for tumor tissue over normal tissue. A similar formulation that contains oxaliplatin instead of cisplatin is LiPloxa. Lipoplatin is a liposomal formulation of cisplatin. Compared with cisplatin, it has higher uptake into tumors in vivo and has significantly fewer and less severe side effects. In certain embodiments, lipoplatin is used in combination with paclitaxel for the first-line treatment of advanced ovarian cancer. In certain embodiments, lipoplatin is used in combination with gemcitabine for pancreatic cancer.

In certain embodiments, the platinum-based therapeutic agent is a nanoparticle formulation with a polymer scaffold (e.g., ProLindac, and polyamidoamine (PAMAM) dendrimer bound platin). Polymer-based nanoparticles for the delivery of platin drugs can come in a variety of forms from polydispersed linear polymers that roughly roll up into a nanoparticle shape, to the high ordered and monodispersed polymers called dendrimers.

For example, ProLindac is a polymer formulation of platin that yields the same active component as oxaliplatin inside of cancer cells. It's made from the highly hydrophilic and biocompatible polymer hydroxypropylmethacrylamide. Attachment of the platin to the polymer is stable at blood serum pH, but upon entering the lower pH environment of a cancer cell, the platin drug is slowly aquated and goes on to bind DNA. ProLindac has undergone a number of phase I and II trials.

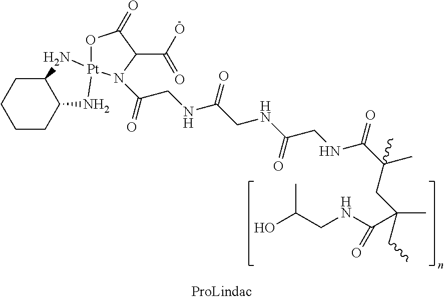

Dendrimers are highly branched synthetic polymers that are made using a step-by-step reaction and are highly useful in drug delivery. They can be synthesized in a variety of sizes, often referred to as generations, which represent the number of branching points in the polymer from the central core. They are able to bind drugs in a variety of ways, including electrostatic interactions, pocket binding and chemical tethering to the dendrimer surface. For platins the most studied and useful are the polyamidoamine (PAMAM) dendrimers. Full generation PAMAM dendrimers have amine surface groups that can be used to irreversibly bind platinum, thus using the dendrimer as part of the structure of the drug rather than as a delivery vehicle. Half-generation PAMAM dendrimers have carboxylate surface groups and can bind cationic platin drugs via electrostatic interactions. Alternatively, the carboxylate groups on the surface of dendrimers can be used to tether the platins. Upon aquation inside cancer cells, the dendrimer releases the active component of cisplatin or oxaliplatin.

In certain embodiments, the dendrimers are without amine groups within the branches. Two examples of such dentrimers are ester and thiol-based dendrimers with terminal hydroxyl or carboxylate groups that can be used as drug delivery vehicles.

In certain embodiments, the platinum-based therapeutic agent is a nanoparticle formulation with a protein scaffold (e.g., transferrin-bound platin). For example, transferrin is a major protein found in blood serum that can act as a delivery vehicle for platins, because many cancer types overexpress transferrin receptors on their cell surfaces. Transferrin is normally responsible for transporting iron around the body and has two high affinity Fe³⁺ sites. When the protein is bound to two iron atoms, it becomes holo-transferrin, and when not bound by iron, it is apo-transferrin. Both forms of the protein can be used as drug delivery vehicles. For example, cisplatin can bind to the hydroxyl group of the threonine 457 residue, which is located in the iron binding pocket, although other binding sites are thought to exist. As such, cisplatin binds the protein competitively with iron, and a higher loading of cisplatin is achieved with apo-transferrin (e.g., 22 cisplatin molecules per protein) compared with holo-transferrin (e.g., 15 cisplatin molecules per protein). In certain embodiments, each transferrin binds less than 15 cisplatin molecules in a subject formulation. In certain embodiments, each transferrin binds about 3-7 cisplatin molecules in a subject formulation.

In certain embodiments, the platinum-based therapeutic agent is a nanoparticle formulation with a metallic nanoparticle scaffold (e.g., gold or iron oxide nanoparticle-bound platin). Nanoparticles made from metals, such as gold, platinum, or iron oxide, can be produced in a variety of shapes, including spheres, rods and pyramids, and bowls. All of these have shown potential as drug delivery vehicles and in other medical applications such as diagnostics and photothermal therapy. Gold and iron oxide nanoparticles have both been examined as delivery vehicles for platin drugs.

In certain embodiments, a platin drug is loaded onto the surface of gold nanoparticles directly. In certain embodiments, a platin drug is tethered to the nanoparticle surface using thiol-based chemical linkers. On solid gold nanoparticles, thiols are known to bind strongly to the surface and in the process form monolayers. In certain embodiments, dithiols are used in the linker to further strengthen the bond of the tether to the nanoparticles. In certain embodiments, a carboxylate functional group is used on the other end of the tether to facilitate attachment of a platin drug. In certain embodiments, a thiol-modified cyclodextrin is attached to the surface of the gold nanoparticle, while a platin drug is modified with an adamantine ligand, which forms a host-guest complex with the cyclodextrin by binding within its cavity. Upon entering the cells, the platin is reduced from platinum(IV) to platinum(II), thus releasing the platin.

In certain embodiments, iron oxide nanoparticles are used in platin drug delivery. As iron remains susceptible to magnetic fields in its oxidized state, such nanoparticles can be actively transported to solid cancers in the body using external magnetic fields. In certain embodiments, the iron oxide nanoparticle is pacified by the addition of a gold coating to reduce reaction and breaking down in vivo, without affecting the magnetic properties of the iron oxide.

In certain embodiments, the platinum-based therapeutic agent is a nanoparticle formulation with a carbon nanotube scaffold. Carbon nanotubes are an allotrope of carbon and are long, cylinder-like molecules. Carbon nanotubes are passively selective for cancer cells due to their size; they can have lengths between 10 and 1000 nm. Such carbon nanotubes can deliver platin drugs in three different ways: i) the drug (e.g., cisplatin, nedaplatin, carboplatin and oxaliplatin) can be stored/encapsulated within the cavity of the nanotube, ii) the drug can be directly attached to the surface of the nanotubes that have been functionalized with carboxylic acid or amine groups, or iii) the drug can be attached through the use of a chemical tether.

In certain embodiments, in open-ended carbon nanotubes, platin drugs are loaded into the cavity of the tubes through simple diffusion. In certain embodiments, in carbon nanohorns (carbon nanotubes with rounded ends that cap the particles), holes are created in the tubes by heating them up to 500° C. before the drug is loaded. Release of platins from the nanotubes is controlled either through diffusion or through the use of iron nanoparticle caps.

In certain embodiments, platins are directly attached to the surface of functionalized carbon nanotubes. In certain embodiments, the tubes are synthesized with surface carboxylate groups, and the platin drugs are attached through direct coordination. In this case, release of the drug is dependent on aquation inside of cancer cells. In certain embodiments, the carbon nanotubes are functionalized with amine surface groups, and a platinum(IV) drug with carboxylate ligands is attached through the formation of an amide bond. Drug release is achieved when the platin is reduced from platinum(IV) to platinum(II) inside the cancer cell.

In certain embodiments, the platin drugs are attached to carbon nanotubes through the use of a tether held to the surface of the tubes through hydrophobic effects. The drug is released within cancer cells when it is reduced to platinum(II).

In certain embodiments, the platinum-based therapeutic agent is actively targeted to a target location with a targeting agent, which selectively recognize and bind to proteins and peptides on the surface of cancer cells. For example, some receptors can be for essential nutrients needed for the growth of the cancer, and are overexpressed on cancer cells (but may not be specific for cancer cells, thus providing pseudo-active targeting). For other receptors that may be unique to individual cancer cells, their targeting provides a very selective drug delivery.

For example, the platin composition may be tethered to a substrate or nutrient including vitamin, steroid, amino acid, and/or sugar. Many cancerous cells overexpress folate receptors on their surface to ensure a high supply of folic acid, which is a vitamin used by cells to synthesize nucleobases for the production of DNA and is thus essential for cell proliferation. Thus in certain embodiments, folate is used as a pseudo-selective targeting agent for many different types of cancers. Folic acid contains two carboxylic acid groups that can be directly coordinated to the active components of cisplatin and oxaliplatin. Alternatively, cisplatin can be conjugated to folic acid through the use of a PEG spacer. In either case, aquation of the drug inside the cell yields the active component of cisplatin. Folate has also been attached to the surface of different delivery vehicles, like carbon nanotubes and micelles, for the delivery of platins.

In certain embodiments, estrogens and the related family of hormones are used as targeting agents in the treatment of several types of cancer, since estrogen receptors are highly expressed in breast (60-70%), uterus (70-73%) and ovarian (60%) cancers, and provide pseudo-selective target for platin drugs. In certain embodiments, the estrogen related hormone is 17β-estradiol, which can be modified to provide accessible amine groups as suitable functional groups for the direct attachment of a platin drug. As the platin is coordinated via amine ligands, the estradiol is not removed during aquation and thus remains a permanent part of the drug upon its binding to DNA.

In certain embodiments, the platin composition is linked to an antibody, or a targeting peptide such as RGD sequence or TAT-fragment. Antibodies and short peptides, especially cell penetrating peptides, provide higher selectivity for cancer cells than what can be achieved using nutrients. For example, trastuzumab is a monoclonal antibody that recognizes and binds HER2 receptors on certain breast cancers. A cisplatin-like platin has been successfully conjugated to trastuzumab to form an antibody-drug complex (ADC). By varying the linker used to tether the platin to the antibody, 2-3 or more drug molecules could be attached to each antibody. In certain embodiments, tethering of platinum to the antibody does not affect the immunoreactivity of the antibody.

In certain embodiments, a range of different targeting peptides are used to improve drug uptake into cells. For example, the 12-residue peptide (TMGFTAPRFPHY), known as PH1, is selective for the tyrosine kinase-based Tie2 receptor that is overexpressed on a number of different cancer cell lines. It can be and has been tethered to the surface of cisplatin containing liposomes to improve the drug's selectivity for cancerous tissue. Integrin receptors can be targeted by peptides that contain Arg-Gly-Asp (RGD) sequences in their structure. A platinum(IV) derivative of picoplatin can be and has been tethered to cyclic and RAFT versions of RGD. An 11-residue fragment of the TAT protein (YGRKKRRQRRR), derived from HIV-1, is useful for carrying drugs across cell membranes. A platinum(IV) derivative of oxaliplatin can be and has been tethered to a TAT fragment using solid-phase chemistry. Either one or two platins can be tethered to a single peptide and both are more active in cancer cells compared with oxaliplatin complexes without the peptide.

In certain embodiments, the platin composition is linked to a targeting aptamer. Aptamers are short (usually 20-100 bases) DNA or RNA strands that bind proteins and peptides with high target selectivity. They are generated through the process of systematic evolution of ligands by exponential enrichment (e.g., SELEX). In many ways aptamers can be superior to antibodies as they are just as selective and have identical binding affinities but are also non-immunogenic, more easily synthesized and can be generated against practically any target. Aptamers can be and have been used as active targeting agents for a number for platin-based drugs. For example, cisplatin has been encapsulated into liposomes to which the AS1411 aptamer was tethered to the surface. This aptamer is specific for nucleolin, which is a cell proliferation protein overexpressed on the surface of many types of cancer cells. In certain embodiments, cholesterol is conjugated to the end of the aptamer to facilitate attachment to the surface of the liposome when it is absorbed into the lipid bilayer. In another example, Aplatinum(IV) complex, similar to LA-12, which yields cisplatin when reduced inside the cell, has been included in liposomes that are actively targeted to prostate cancer cells via the attachment of the prostate specific membrane antigen selective aptamer called A10. For this delivery system, the aptamer is attached to the surface of the liposome through an amide bond between the carboxylate groups on the liposome surface and an amine attached to the 3′ end of the aptamer.

In certain embodiments, inert platin compounds, such as phenanthroline-based platins, can be used as potential drugs, since these agents are capable of reversibly intercalating between the base-pairs in double stranded DNA structures such as those available in many aptamers.

In certain embodiments, the aptamer is sgc8c, which folds to form regions of double helical DNA in solution, and which is selective for T cell leukemia. This aptamer forms a loop and stem structure into which the platin complex, PHENEN, can be intercalated for simultaneous targeting and drug delivery.

In certain embodiments, the platinum-based therapeutic agent is any one described in Apps et al., The state-of-play and future of platinum drugs. Endocrine-Related Cancer 22:R219-R233, 2015 (incorporated herein by reference).

In certain embodiments, the method further comprises administering to the patient a PARPi (Poly(ADP-ribose) polymerase inhibitor, such as CEP-6800).

In certain embodiments, the method further comprises selecting the patient for treatment based on the presence of the TDP cancer in the patient.

In certain embodiments, the patient is a human, a non-human primate, a non-human mammal, a rodent (e.g., rat, mouse, hamster, rabbit, etc.), a farm/livestock animal (cattle, horse, goat, sheep, pig, camel etc.).

Another aspect of the invention provides a method of identifying and selecting a cancer patient suffering from triple negative breast cancer, ovarian cancer, hepatocellular carcinoma, or endometrial carcinoma, as a candidate suitable for a platinum-based therapy, the method comprising:
(a) obtaining a tumor sample from the cancer patient;
(b) determining tandem duplications of the tumor sample;
(c) determining a TDP Score using Formula (I):

$$TDP Score = TDP Raw Score + k = - \frac{{Σ}_{i} \vert {Obs}_{i} - {Exp}_{i} \vert}{TD} + k \qquad ( I )$$

wherein:
   TD is total number of tandem duplications,
   Obsᵢ is observed number of tandem duplications for each chromosome i,
   Expᵢ is expected number of tandem duplications for each chromosome i, and,
   k is 0.71; and,
(d) identifying and selecting the patient as a candidate for the treatment of a platinum-based therapeutic agent, when the TDP Score is positive.

A related aspect of the invention provides a method of identifying (and selecting) a patient as a candidate for a platinum-based therapy for a cancer of the patient, the method comprising obtaining a TDP Score, based on Formula (I), of the cancer of the patient, and selecting the patient as the candidate for a platinum-based therapy if the TDP Score is positive, or is indicative of a genomic configuration characterized by tandem duplications (TDs) evenly distributed across all chromosomes.

In certain embodiments, the TDP Score is not indicative of a localized segmental amplifications with tandem duplications (TDs).

In certain embodiments, the distribution of the TDP Raw Score follows a trimodal pattern with a 1^{st}, a 2^{nd}, and a 3^{rd} mode, each mode independently having a peak and a standard deviation, and k equals the absolute value of the sum of the peak 2^{nd} mode TDP Raw Score and two standard deviations (SD) of the 2^{nd} mode.

In certain embodiments, the genomic configuration of the cancer, such as tandem duplication, is determined based on whole-genome sequencing (WGS) data, or SNP-array data (such as Affymetrix SNP 6.0 array data), or both. For example, the whole-genome sequencing (WGS) may be performed using Next Generation Sequencing (NGS), such as Next Generation Sequencing performed using Illumia HisSeq 2500 platform.

In certain embodiments, the cancer is a triple negative breast cancer (TNBC), an ovarian cancer (e.g., a serous ovarian cancer), a hepatocellular carcinoma, or an endometrial carcinoma (e.g., a uterine corpus endometrial carcinoma (UCECs), or a cluster 4 endometrial carcinoma).

In certain embodiments, the cancer is not a prostate cancer, not a glioblastoma, and not a non-triple negative breast cancer (NTBC).

In certain embodiments, the cancer is an adrenocortical carcinoma, esophageal carcinoma, stomach adeno-carcinoma, lung squamous cell, or pancreatic adeno-carcinomas.

In certain embodiments, the patient suffering from the cancer has not been treated previously with a chemotherapeutic agent.

In certain embodiments, the patient suffering from the cancer has been treated with a chemotherapeutic agent other than a platinum-based therapeutic agent.

In certain embodiments, the platinum-based therapy comprises cisplatin, carboplatin, satraplatin, picoplatin, oxaliplatin, nedaplatin, lobaplatin, heptaplatin, Triplatin, LA-12, dicycloplatin, phosphaplatin, phenanthriplatin, any one or more of the above encapsulated within a macrocycle (such as cucurbit[n]urils, n-cyclodextrins and calix[n]arenes), a nanoparticle formulation thereof with a micelle/liposome (e.g., Aroplatin, SPI-77, LiPlaCis, Lipoplatin), polymer (e.g., ProLindac, and polyamidoamine (PAMAM) dendrimer bound platin), protein (e.g., transferrin-bound platin), metallic nanoparticle (e.g., gold or iron oxide nanoparticle-bound platin), or carbon nanotube scaffold, and/or an actively targeted platin thereof (such as platins tethered to a substrate or nutrient including vitamin, steroid, amino acid, and sugar; platins linked to an antibody or targeting peptide such as RGD sequence or TAT-fragment; and platins linked to a targeting aptamer).

In certain embodiments, the platinum-based therapeutic agent comprises cisplatin, carboplatin, oxaliplatin, nedaplatin, heptaplatin, lobaplatin, satraplatin, picoplatin, triplatin tetranitrate, phenanthriplatin, or a combination thereof.

In certain embodiments, the platinum-based therapeutic agent comprises cisplatin.

In certain embodiments, the platinum-based therapeutic agent comprises carboplatin.

In certain embodiments, the method further comprises administering a therapeutically effective amount of the platinum-based therapy to the cancer patient. In certain embodiments, the platinum-based therapy comprising cisplatin, carboplatin, satraplatin, picoplatin, oxaliplatin, nedaplatin, lobaplatin, heptaplatin, Triplatin, LA-12, dicycloplatin, phosphaplatin, phenanthriplatin, any one or more of the above encapsulated within a macrocycle (such as cucurbit[n]urils, n-cyclodextrins and calix[n]arenes), a nanoparticle formulation thereof with a micelle/liposome (e.g., Aroplatin, SPI-77, LiPlaCis, Lipoplatin), polymer (e.g., ProLindac, and polyamidoamine (PAMAM) dendrimer bound platin), protein (e.g., transferrin-bound platin), metallic nanoparticle (e.g., gold or iron oxide nanoparticle-bound platin), or carbon nanotube scaffold, and/or an actively targeted platin thereof (such as platins tethered to a substrate or nutrient including vitamin, steroid, amino acid, and sugar; platins linked to an antibody or targeting peptide such as RGD sequence or TAT-fragment; and platins linked to a targeting aptamer).

In certain embodiments, the platinum-based therapeutic agent comprises cisplatin, carboplatin, oxaliplatin, nedaplatin, heptaplatin, lobaplatin, satraplatin, picoplatin, triplatin tetranitrate, phenanthriplatin, or a combination thereof.

In certain embodiments, the platinum-based therapeutic agent comprises cisplatin.

In certain embodiments, the platinum-based therapeutic agent comprises carboplatin.

In certain embodiments, the method further comprises administering to the patient a PARPi (Poly(ADP-ribose) polymerase inhibitor, such as CEP-6800).

Yet another aspect of the invention provides a method of predicting the outcome of a cancer treatment in a patient having a cancer, wherein the cancer treatment comprises administering a therapeutically effective amount of a platinum-based therapeutic agent to the patient, the method comprising: determining a TDP Score, based on Formula (I), of the cancer, wherein a positive TDP Score is indicative of a favorable outcome, and a negative TDP Score is indicative of an unfavorable outcome.

In certain embodiments, the method further comprises administering, or continuing to administer, the therapeutically effective amount of the platinum-based therapeutic agent to the patient if the TDP Score is positive.

In certain embodiments, the method further comprises discontinuing further treatment of the patient with the platinum-based therapeutic agent if the TDP Score is negative.

It should be understood that any of the embodiment described herein, including those only described under one aspect of the invention or only in the Example section, can be readily combined with any one or more other such embodiments, unless such combination is explicitly disclaimed or inapplicable (e.g., mutual exclusive).

### Next Generation Sequencing

As used herein, “Next Generation Sequencing (NGS),” also used interchangeably with “high-throughput sequencing (HTP),” refers to a collection of modern sequencing methods capable of producing large amount of sequencing information over a short period of time, compared to tradition DNA sequencing methods such as the Sanger sequencing (“DNA polymerase based chain-termination sequencing”) and the Maxam-Gilbert sequencing methods (“DNA sequencing by chemical degradation”).

HTP/NGS sequencing is usually used in genome-scale sequencing, resequencing, transcriptome profiling (RNA-Seq), DNA-protein interactions (ChIP-sequencing), and epigenome characterization. Such sequencing technologies may have different mechanisms, but all are capable of producing thousands or millions of sequences concurrently by, for example, parallelizing the sequencing process. High-throughput sequencing technologies are intended to lower the cost of DNA sequencing beyond what is possible with the traditional Sanger dye-terminator methods. Some high-throughput sequencing methods carry out as many as 500,000 sequencing-by-synthesis operations run in parallel.

In certain embodiments, the HTP/NGS is massively parallel signature sequencing (MPSS). MPSS is the first of the high-throughput sequencing technologies developed in the 1990s at Lynx Therapeutics. It was a bead-based method that used a complex approach of adapter ligation followed by adapter decoding, reading the sequence in increments of four nucleotides. This method made it susceptible to sequence-specific bias or loss of specific sequences. MPSS eventually led to the development of the simpler sequencing-by-synthesis approach, and the essential properties of the MPSS output are typical of later high-throughput data types, including hundreds of thousands of short DNA sequences.

In certain embodiments, the HTP/NGS is polony sequencing, which combined an in vitro paired-tag library with emulsion PCR, an automated microscope, and ligation-based sequencing chemistry to sequence an E. coli genome in 2005, at an accuracy of >99.9999%, and a cost approximately 1/9 that of Sanger sequencing. The technology was eventually incorporated into the Applied Biosystems (acquired by Life Technologies, now part of Thermo Fisher Scientific) SOLiD platform.

In certain embodiments, the HTP/NGS is 454 pyrosequencing, a parallelized version of pyrosequencing initially developed by 454 Life Sciences (acquired by Roche Diagnostics). The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

In certain embodiments, the HTP/NGS is Illumina (Solexa) sequencing, such as Illumina (Solexa) sequencing performed on an Illumina HiSeq 2500 sequencer, or an Illumina MiSeq sequencer.

Illumina (Solexa) sequencing is a HTP/NGS sequencing method based on reversible dye-terminators technology, and engineered polymerases. It is partly based on the reversible terminated chemistry concept, and a version of the massivelly parallel sequencing technology based on “DNA Clusters” or “DNA colonies” which involves the clonal amplification of DNA on a surface. In this method, DNA molecules and primers are first attached on a slide or flow cell, and amplified with polymerase so that local clonal DNA colonies, later coined “DNA clusters,” are formed. To determine the sequence, four types of reversible terminator bases (RT-bases) are added and non-incorporated nucleotides are washed away. A camera takes images of the fluorescently labeled nucleotides. Then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). For example, in 2012, with cameras operating at more than 10 MHz A/D conversion rates and available optics, fluidics and enzymatics, throughput can be multiples of 1 million nucleotides/second, corresponding roughly to 1 human genome equivalent at 1× coverage per hour per instrument, and 1 human genome re-sequenced (at approx. 30×) per day per instrument (equipped with a single camera).

In certain embodiments, the HTP/NGS is SOLiD sequencing, which employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. Commercial systems for SOLiD sequencing are available by Applied Biosystem (now a Life Technologies brand).

In certain embodiments, the HTP/NGS is ion torrent semiconductor sequencing, developed by Ion Torrent Systems Inc. (now owned by Life Technologies) as a system based on using standard sequencing chemistry, but with a novel, semiconductor based detection system. This method of sequencing is based on the detection of hydrogen ions that are released during the polymerisation of DNA, as opposed to the optical methods used in other sequencing systems. A microwell containing a template DNA strand to be sequenced is flooded with a single type of nucleotide. If the introduced nucleotide is complementary to the leading template nucleotide, it is incorporated into the growing complementary strand. This causes the release of a hydrogen ion that triggers a hypersensitive ion sensor, which indicates that a reaction has occurred. If homopolymer repeats are present in the template sequence, multiple nucleotides will be incorporated in a single cycle. This leads to a corresponding number of released hydrogens and a proportionally higher electronic signal.

In certain embodiments, the HTP/NGS is DNA nanoball sequencing, a type of high throughput sequencing technology used to determine the entire genomic sequence of an organism. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other high-throughput sequencing platforms. However, only short sequences of DNA are determined from each DNA nanoball which makes mapping the short reads to a reference genome somewhat more difficult. This technology has been used for multiple genome sequencing projects.

In certain embodiments, the HTP/NGS is Heliscope single molecule sequencing, a method of single-molecule sequencing developed by Helicos Biosciences (bankrupt since 2009). It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are short, averaging 35 bp, and are performed by the Heliscope sequencer. In 2009, a human genome was sequenced using the Heliscope.

In certain embodiments, the HTP/NGS is Single molecule real time (SMRT) sequencing, based on the sequencing by synthesis approach. In this method, the DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide upon its incorporation into the DNA strand, leaving an unmodified DNA strand. According to Pacific Biosciences, the SMRT technology developer, this methodology allows detection of nucleotide modifications (such as cytosine methylation), through the observation of polymerase kinetics. This approach allows reads of 20,000 nucleotides or more, with average read lengths of 5 kilobases. In 2015, Pacific Biosciences announced the launch of a new sequencing instrument called the Sequel System, with 1 million ZMWs compared to 150,000 ZMWs in the PacBio RS II instrument.

In certain embodiments, the HTP/NGS is Nanopore DNA sequencing. In this method, the DNA passing through the nanopore changes its ion current. This change is dependent on the shape, size and length of the DNA sequence. Each type of the nucleotide blocks the ion flow through the pore for a different period of time. The method does not require modified nucleotides and is performed in real time.

Early industrial research into this method was based on a technique called “Exonuclease sequencing,” where the readout of electrical signals occurring at nucleotides passing by alpha-hemolysin pores covalently bound with cyclodextrin. However, the subsequently commercial method, “strand sequencing” sequencing DNA bases in an intact strand.

Two main areas of nanopore sequencing in development are solid state nanopore sequencing, and protein based nanopore sequencing. Protein nanopore sequencing utilizes membrane protein complexes such as ∝-Hemolysin, MspA (Mycobacterium Smegmatis Porin A) or CssG, which show great promise given their ability to distinguish between individual and groups of nucleotides. In contrast, solid-state nanopore sequencing utilizes synthetic materials such as silicon nitride and aluminum oxide and it is preferred for its superior mechanical ability and thermal and chemical stability. The fabrication method is essential for this type of sequencing given that the nanopore array can contain hundreds of pores with diameters smaller than eight nanometers.

The concept originated from the idea that single stranded DNA or RNA molecules can be electrophoretically driven in a strict linear sequence through a biological pore that can be less than eight nanometers, and can be detected given that the molecules release an ionic current while moving through the pore. The pore contains a detection region capable of recognizing different bases, with each base generating various time specific signals corresponding to the sequence of bases as they cross the pore which are then evaluated. Precise control over the DNA transport through the pore is crucial for success. Various enzymes such as exonucleases and polymerases have been used to moderate this process by positioning them near the pore's entrance.

Other than the above described relatively mature HTP/NGS sequencing methods, a number of relatively less mature and potentially more advanced HTP/NGS sequencing methods may also be used in the instant invention. Such methods may include solid-state nanopores (a version of the now commercialized Nanopore DNA sequencing); microscopy-based techniques, such as atomic force microscopy or transmission electron microscopy that are used to identify the positions of individual nucleotides within long DNA fragments (>5,000 bp) by nucleotide labeling with heavier elements (e.g., halogens) for visual detection and recording; and third generation technologies aiming to increase throughput and decrease the time to result and cost by eliminating the need for excessive reagents and harnessing the processivity of DNA polymerase. These HTP/NGS methods are described below.

In certain embodiments, the HTP/NGS is tunnelling currents DNA sequencing, which is another approach using measurements of the electrical tunnelling currents across single-strand DNA as it moves through a channel. Depending on its electronic structure, each base affects the tunnelling current differently, allowing differentiation between different bases. The use of tunnelling currents has the potential to sequence orders of magnitude faster than ionic current methods. The sequencing of several DNA oligomers and micro-RNA has already been achieved.

In certain embodiments, the HTP/NGS is sequencing by hybridization, a non-enzymatic method that uses a DNA microarray. In this method, a single pool of DNA to be sequenced is fluorescently labeled and hybridized to an array containing known sequences. Strong hybridization signals from a given spot on the array identifies its sequence in the DNA being sequenced. This method of sequencing utilizes binding characteristics of a library of short single stranded DNA oligonucleotides or DNA probes, to reconstruct a target DNA sequence. Non-specific hybrids are removed by washing, and the target DNA is eluted. Hybrids are re-arranged such that the DNA sequence can be reconstructed. The benefit of this sequencing type is its ability to capture a large number of targets with a homogenous coverage. A large number of chemicals and starting DNA is usually required. However, with the advent of solution-based hybridization, much less equipment and chemicals are necessary.

In certain embodiments, the HTP/NGS is sequencing with mass spectrometry. Matrix-assisted laser desorption ionization time-of-flight mass spectrometry, or MALDI-TOF MS, has specifically been investigated as an alternative method to gel electrophoresis for visualizing DNA fragments. With this method, DNA fragments generated by chain-termination sequencing reactions are compared by mass rather than by size. The mass of each nucleotide is different from the others, and this difference is detectable by mass spectrometry. Single-nucleotide mutations in a fragment can be more easily detected with MS than by gel electrophoresis alone. MALDI-TOF MS can more easily detect differences between RNA fragments, so researchers may indirectly sequence DNA with MS-based methods by converting it to RNA first.

In certain embodiments, the HTP/NGS is microfluidic Sanger sequencing. In this method, the entire thermocycling amplification of DNA fragments as well as their separation by electrophoresis is done on a single glass wafer (approximately 10 cm in diameter), thus reducing the reagent usage as well as cost. In some instances, the throughput of conventional sequencing can be increased through the use of microchips.

In certain embodiments, the HTP/NGS is microscopy-based techniques. This approach directly visualizes the sequence of DNA molecules using electron microscopy. The first identification of DNA base pairs within intact DNA molecules by enzymatically incorporating modified bases, which contain atoms of increased atomic number, direct visualization and identification of individually labeled bases within a synthetic 3,272 base-pair DNA molecule and a 7,249 base-pair viral genome has been demonstrated.

In certain embodiments, the HTP/NGS is RNAP sequencing. This method is based on the use of RNA polymerase (RNAP) attached to a polystyrene bead. One end of the DNA to be sequenced is attached to another bead, with both beads being placed in optical traps. RNAP motion during transcription brings the beads in closer and their relative distance changes, which can then be recorded at a single nucleotide resolution. The sequence is deduced based on the four readouts with lowered concentrations of each of the four nucleotide types, similarly to the Sanger method. A comparison is made between regions, and sequence information is deduced by comparing the known sequence regions to the unknown sequence regions.

In certain embodiments, the HTP/NGS is in vitro virus high-throughput sequencing. This is a method developed to analyze full sets of protein interactions using a combination of 454 pyrosequencing and an in vitro virus mRNA display method. Specifically, this method covalently links proteins of interest to the mRNAs encoding them, then detects the mRNA pieces using reverse transcription PCRs. The mRNA may then be amplified and sequenced. The combined method was titled IVV-HiTSeq and can be performed under cell-free conditions.

With the general embodiments of the invention described above, the Examples below serve to further illustrate certain non-limiting specific embodiments of the invention.

### EXAMPLES

Provided in this example is the first detailed molecular characterization of a distinct cancer genomic configuration, the tandem duplicator phenotype (TDP), that is significantly enriched in certain cancers, such as the molecularly related TNBC, serous ovarian and endometrial carcinomas. Data presented herein shows that TDP represents an oncogenic configuration featuring: (i) genome-wide disruption of cancer genes; (ii) loss of cell cycle control and DNA damage repair; and, (iii) increased sensitivity to cisplatin chemotherapy both in vitro and in vivo. Therefore, the TDP is a systems strategy to achieve a pro-tumorigenic genomic configuration by altering a large number of oncogenes and tumor suppressors. The TDP arises in a molecular context of joint genomic instability and replicative drive, and is consequently associated with enhanced sensitivity to cisplatin.

### Example 1 Homogeneous Distribution of TDs Across Cancer Genomes as a Systematic Measure of the TDP

Previous attempts at describing the genomic features of the TDP have relied on a basic TD count or on the proportion of TDs relative to the total number of structural variations in a cancer genome. These approaches lack in robustness, as they are prone to be influenced by observer and technical biases, such as sequencing coverage, and are not able to discriminate between the genome-wide TD prevalence that characterizes the TDP versus the abnormal TD accumulation in a few functional genomic loci, previously described in association with focal amplification. To address this problem, a reproducible metric of TD genomic distribution was developed, which is referred to herein as “TDP score.”

For each tumor sample, the total number of TDs mapped by breakpoint analysis were tallied, and then observed (Obsᵢ) and expected number of TDs (Expᵢ) for each chromosome i was compared:

$$TDP Score = - \frac{{Σ}_{i} \vert {Obs}_{i} - {Exp}_{i} \vert}{TD} + k$$

where k equals the threshold value which normalizes all values to the subsequently determined threshold for the TDP configuration (see below).

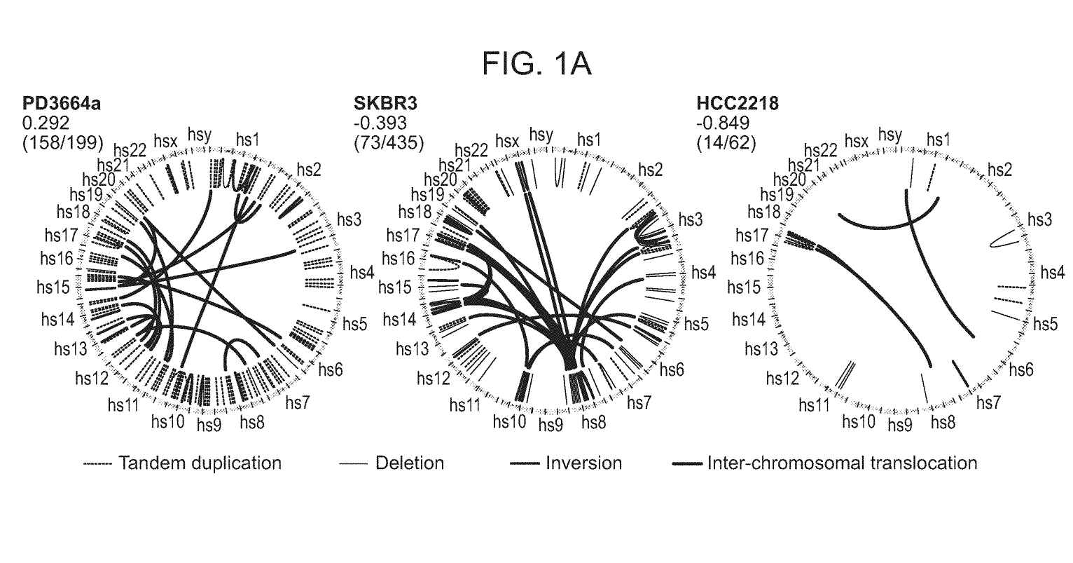

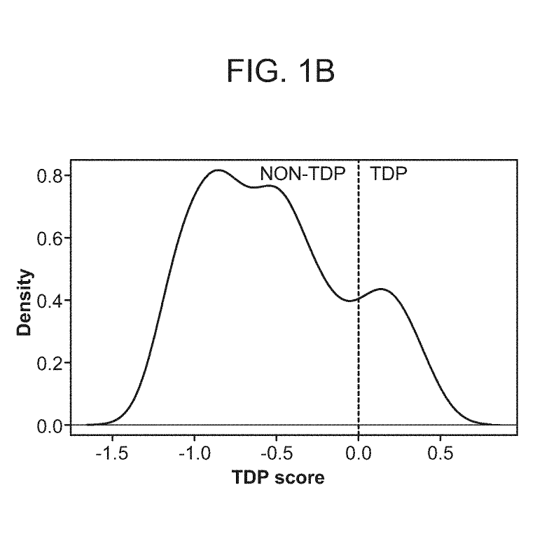

This metric is able to easily distinguish between a genomic configuration characterized by localized segmental amplifications with TDs versus the TDP, in which TDs are evenly distributed across all chromosomes (FIG. 1A).

To address the incidence and genomic properties of the TDP, whole-genome sequencing (WGS) data were combined from 277 human genomes representing 11 cancer types, including 96 breast tumors and cancer cell lines. It was observed that the TDP score distribution in this dataset follows a trimodal pattern (FIG. 6A), suggesting that cancers can be separated into distinct groups based on their propensity for TD formation. Upon visual inspection of tumors within a range of TDP scores by Circos plots, those tumors with the highest scores show the characteristic TD distribution of the TDP (FIG. 1A). In order to derive an unbiased threshold for classifying TDP tumors, the threshold was identified as the score that corresponds to two standard deviations from the second modal peak (−0.71, FIG. 6A). To simplify data presentation, the TDP score was set to zero at this defining threshold (k), resulting in positive and negative scores for TDP and non-TDP tumors, respectively (FIG. 1B). Using this threshold, 18.1% of the tumors analyzed are classified as TDPs, each showing a high number of TDs (average number of TD per sample=112.2 (23 to 416 range), modal TDP score=0.19) that are broadly distributed throughout the genome (FIG. 1A and FIGS. 6A and 6B). By contrast, non-TDP samples are either associated with an intermediate number of TDs (10 to ˜100, modal TDP score=−0.50) that are invariably clustered in specific genomic regions, or have a low number of TDs altogether (<20) indicative of a more stable genome (FIGS. 1A, 6A, and 6B).

TABLE 1
Prevalence of the TDP among different tumor types

| | Whole-genome Sequencing (WGS) | | | | | SNP-Array | | | | |
|---|---|---|---|---|---|---|---|---|---|---|
| Cancer Type | Total | TDP | % | P | | Total | TDP | % | P | |
| TNBC | 40 | 17 | 42.5 | 2.14E−04 | (E) | 94 | 37 | 39.4 | 1.23E−08 | (E) |
| non-TNBC | 56 | 6 | 10.7 | 5.27E−02 | (D) | 594 | 22 | 3.7 | 2.41E−20 | (D) |
| CA | 14 | 0 | 0 | 6.11E−02 | (ns) | 545 | 6 | 1.1 | 3.36E−31 | (D) |
| G | 16 | 0 | 0 | 4.10E−02 | (D) | 18 | 2 | 11.1 | 2.50E−01 | (ns) |
| HC | 19 | 7 | 36.8 | 2.92E−02 | (E) | NA | — | — | — | — |
| KRCCC | 3 | 0 | 0 | 5.49E−01 | (ns) | 509 | 2 | 0.4 | 4.61E−34 | (D) |
| LA | 25 | 3 | 12.0 | 1.69E−01 | (ns) | NA | — | — | — | — |
| LSCC | 18 | 5 | 27.8 | 1.24E−01 | (ns) | 364 | 31 | 8.5 | 3.43E−05 | (D) |
| MM | 7 | 0 | 0 | 2.47E−01 | (ns) | NA | — | — | — | — |
| OC | 26 | 8 | 30.8 | 4.95E−02 | (E) | 382 | 236 | 61.8 | 4.16E−94 | (E) |
| PC | 43 | 1 | 2.3 | 1.77E−03 | (D) | NA | — | — | — | — |
| EC | 10 | 3 | 30.1 | 1.76E−01 | (ns) | 481 | 123 | 25.6 | 2.80E−09 | (E) |
| Total | 277 | 50 | 18.1 | | | 2987 | 459 | 15.4 | | |

TDP status was assigned based on either whole-genome sequencing data (n = 277 tumor samples) or Affymetrix SNP 6.0 array data (SNP-array, n = 2,987 tumor samples).
P-values were computed using the binomial test.
E, enrichment; D, depletion; ns, non significant.
* Tumor samples were classified based on the stringent thresholds described in FIG. 7E.
TNBC: Triple negative breast cancer; CA: Colorectal adenocarcinoma; G: Glioblastoma; HC: Hepatocellular carcinoma; KRCCC: Kidney renal clear cell carcinoma; LA: Lung adenocarcinoma; LSCC: Lung squamous cell carcinoma; MM: Multiple myeloma; OC: Ovarian cancer; PC: prostate cancer; EC: Endometrial carcinoma.

A similar scoring method was applied to the three other basic rearrangements (deletion, inversion, and inter-chromosomal translocation), but found no evidence for distinct groups to manifest in multimodal score distributions as seen for TDs (FIG. 6C). This suggests that the TDP is not merely an indicator of genomic instability, but instead represents a unique subgroup with a distinct structural phenotype.

Previous evidence has suggested a higher frequency of the TDP in triple negative breast cancer (TNBC) and ovarian cancer. Using the more precise and quantitative TDP measure based on only the WGS dataset, the TDP was confirmed to occur statistically more frequently in TNBC, ovarian cancer and hepatocellular carcinoma, but it is significantly depleted in non-triple negative breast cancer, glioblastoma and prostate cancer (Table 1). Indeed, TDP samples were rarely observed in prostate cancer, in which chromoplexy and chromothripsis appear to be the predominant whole-genome rearrangement patterns. This suggests that different mechanisms are active in different tumor types to produce specific dominant cancer genomic configurations.

While the TDP score is based on the identification of TDs through the assignment of breakpoints, and relies on the availability of WGS data, Ng et al. estimated the prevalence of the TDP by counting the number of TD-like features from array-based copy number profiling in high-grade serous ovarian carcinoma. To compare the performance of the TDP scoring algorithm when applied to sequence versus array-based detection systems, Affymetrix SNP 6.0 array segmented copy number data was analyzed from a subset of 81 tumor genomes profiled as part of The Cancer Genome Atlas (TCGA) project to compute copy number (array)-derived TDP scores and compare them to those obtained using paired-end WGS data (FIGS. 7A and 7B). Using SNP-array copy number data alone, TDP samples could be identified with high specificity (0.95, FIGS. 7C and 7D), but lower sensitivity (0.57), likely due to the lower resolution of array data in detecting short segmental duplications. To increase the discrimination power of the SNP-array-based TDP classification, a more stringent threshold was set to categorize non-TDP samples (FIG. 7E) and improve the sensitivity of the technology to 0.80 (FIG. 7F).

The advantage of analyzing array-based data is the availability of a larger number of cancer samples. When 2,987 primary tumors were classified from several TCGA datasets profiled using the Affymetrix SNP 6.0 array, it was possible to reproduce the previous findings that the TDP is significantly enriched in TNBC (P=1.23E-08) and in ovarian cancers (OV, P=4.16E-94), while being depleted in non-TNBC (P=2.41E-20) (Table 1 and data not shown). In addition, because of the greater number of available tumors in the TCGA array dataset, it was found that endometrial carcinomas (UCECs) also are enriched in TDPs (P=2.80E-09). Interestingly, most of the UCEC samples classified as TDPs belong to the recently described cluster 4 endometrial carcinoma subtype, which is characterized by an extensive degree of copy number variations and has been shown to share a similar molecular phenotype with TNBC and ovarian carcinoma. The consistent observation of TDP enrichment/depletion across alternative cancer data sets, generated via diverse genomic technologies and analysis protocols suggests that the subject scoring approach is reproducible and generalizable.

### Example 2 TD Breakpoints Occur in Regions of Open Chromatin and Active Transcription

To investigate possible molecular mechanisms for the generation of the TDP, the genetic, epigenetic and transcriptional configurations of the chromosomal coordinates affected by TD events in TDP genomes were examined. The analysis focused on breast cancer (TNB and NTNB WGS datasets, n=23 TDP tumor genomes), since this was the best-represented tumor type in the WGS sample cohort and therefore provided adequate statistical power. But the method is not so limited.

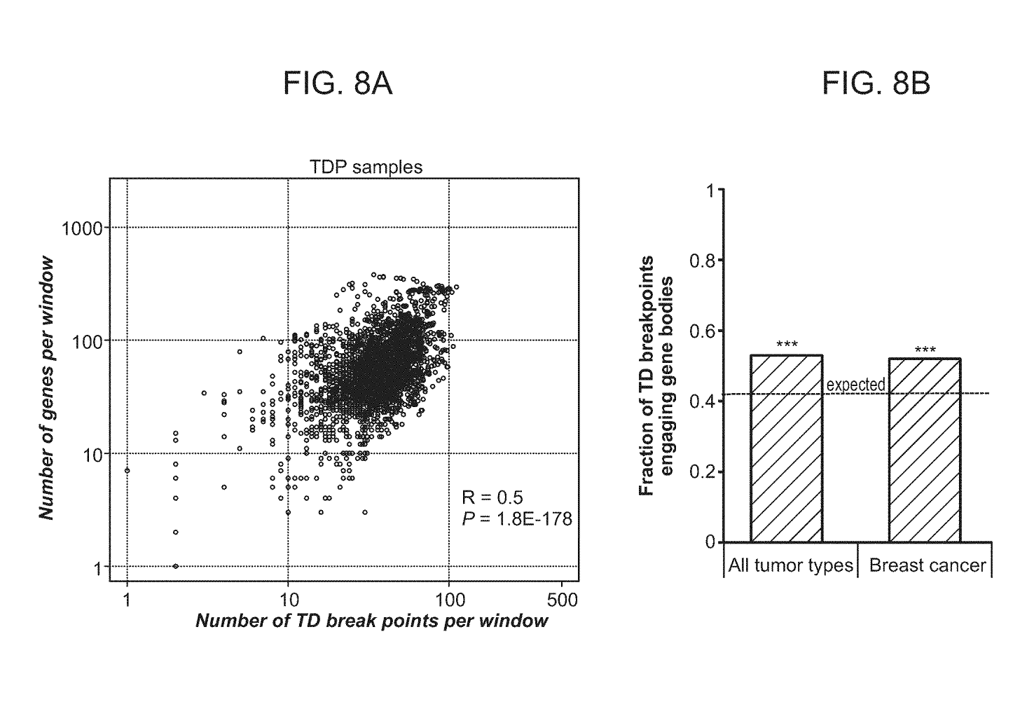

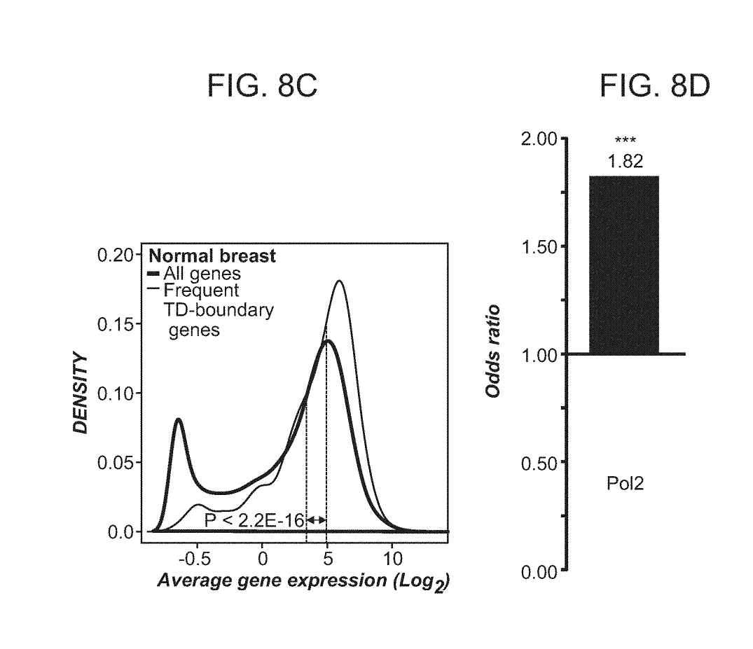

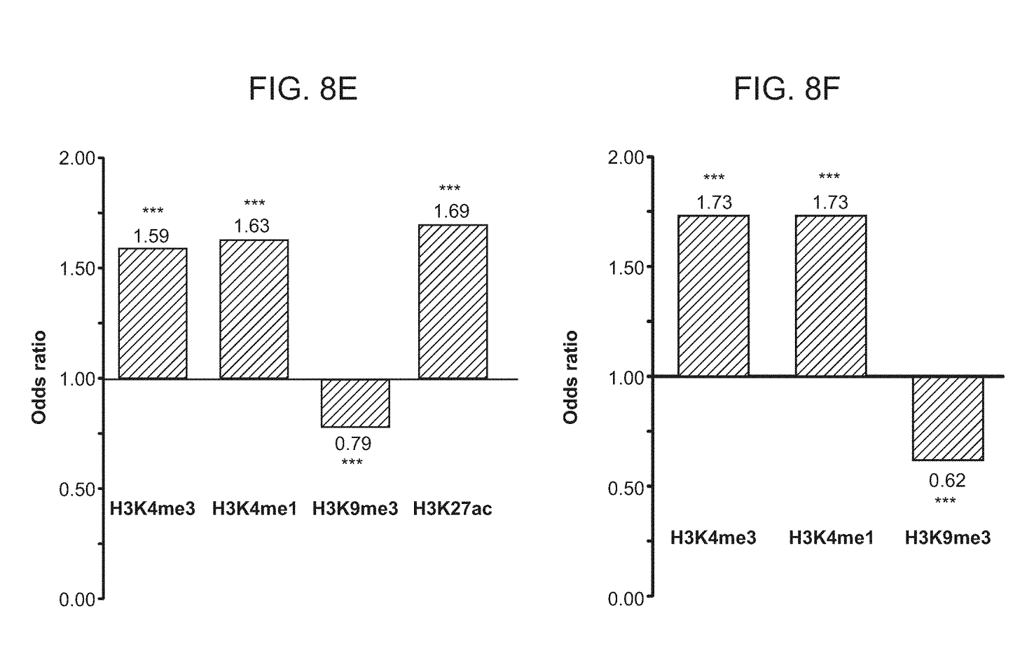

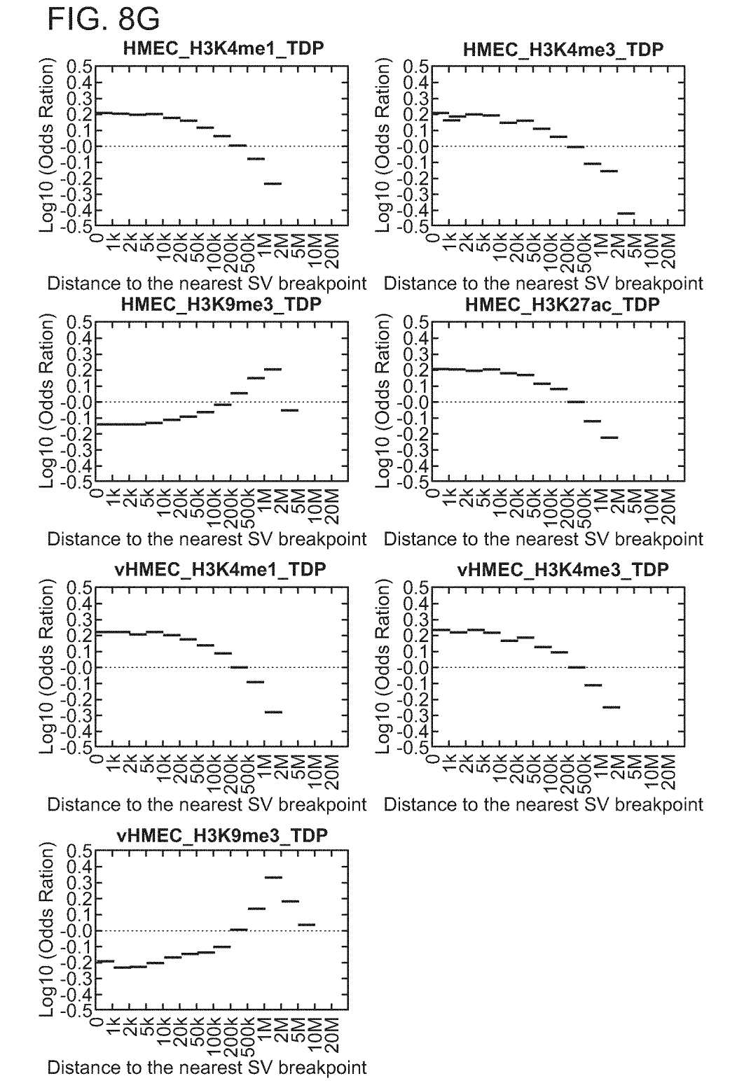

Concerning whether TDs in TDP occurred in functional regions of the genome enriched for genes, a highly significant positive correlation was observed between the number of TD breakpoints and the number of genes in local windows along the genome, (R=0.5, P=1.8E-178; 10 Mb sliding windows, 1 Mb offset) (FIG. 8A). Furthermore, TD breakpoints were biased to occur within gene bodies (exons+introns) as opposed to intergenically (FIG. 8B). The physiological expression levels of genes that are frequently affected by breast cancer TD breakpoints in the normal breast tissue were assessed. Based on a collection of 106 normal breast epithelium samples from the TCGA breast cancer dataset, genes located at the boundaries of TDs show significantly higher levels of activity in the normal breast when compared to the entire gene population (P<2.2E-16) (FIG. 8C). This observation is consistent with the positioning of TD boundaries near genes with anti-oncogenic signals, which would subsequently be disrupted during TD formation. However, it also suggests that TD formation requires transcriptional activity. Indeed, a significant enrichment of Pol2 binding sites as well as of histone modification marks associated with an open chromatin configuration (H3K4me3, H3K4me1 and H3K27ac) in the proximity of TD breakpoints (FIGS. 8D, 8E and 8F) were observed. This is in agreement with recent findings describing a strong affinity of structural variation breakpoints for genomic regions characterized by protein binding and euchromatin. By contrast, H3K9me3 signals, which mark heterochromatin, were depleted from TD breakpoint regions (FIGS. 8E and 8F). Concordant results were obtained by testing different non-overlapping symmetrical windows around the TD breakpoints, showing that significant associations between functionalized chromatin regions and TDs are maintained up to ˜200-500 Kb from the TD breakpoints (FIG. 8G). Overall, these results concordantly indicate a significantly higher likelihood for TD breakpoints to affect transcriptionally active, easily accessible chromatin regions.

### Example 3 Genomic Features of TDs in TDP and Non-TDP Tumors

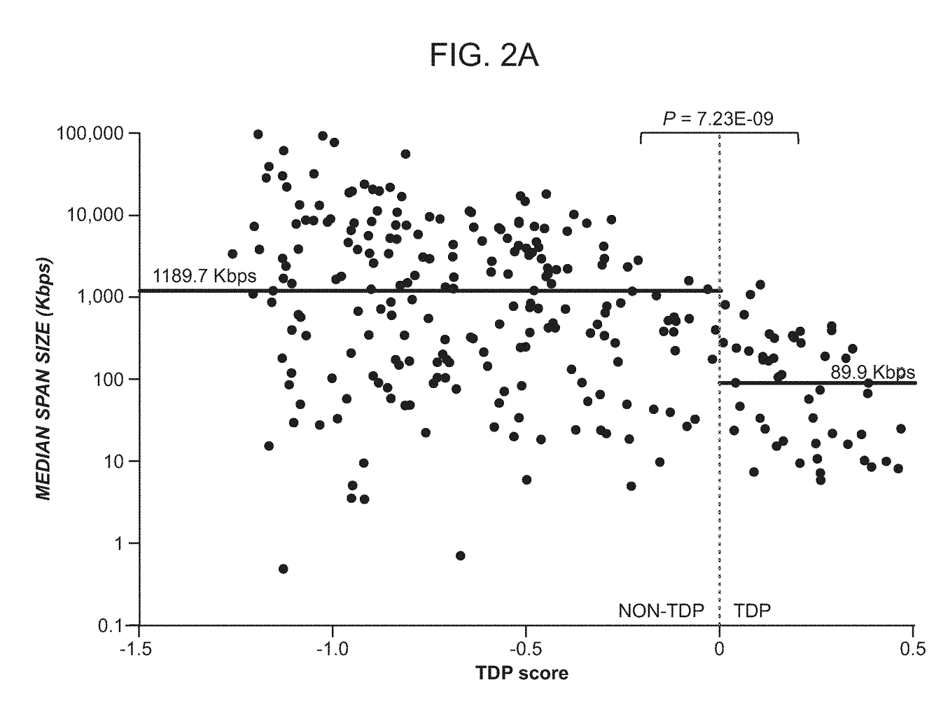

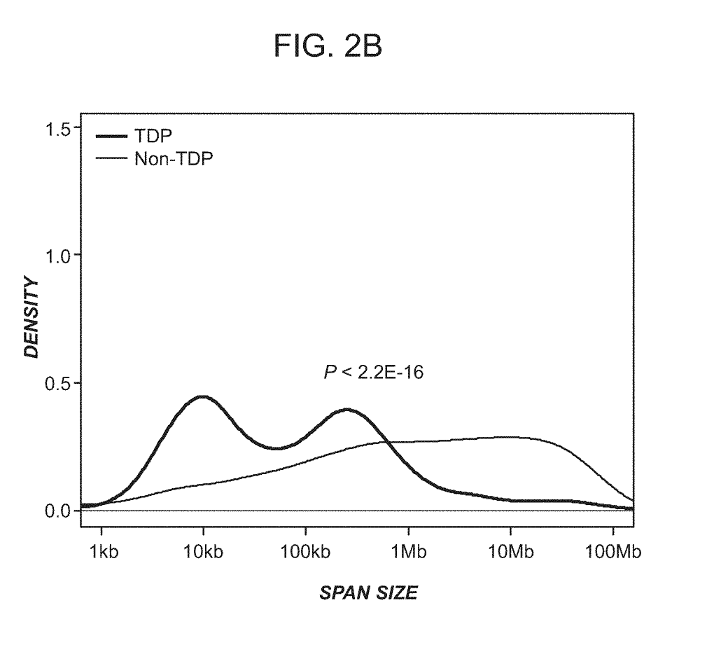

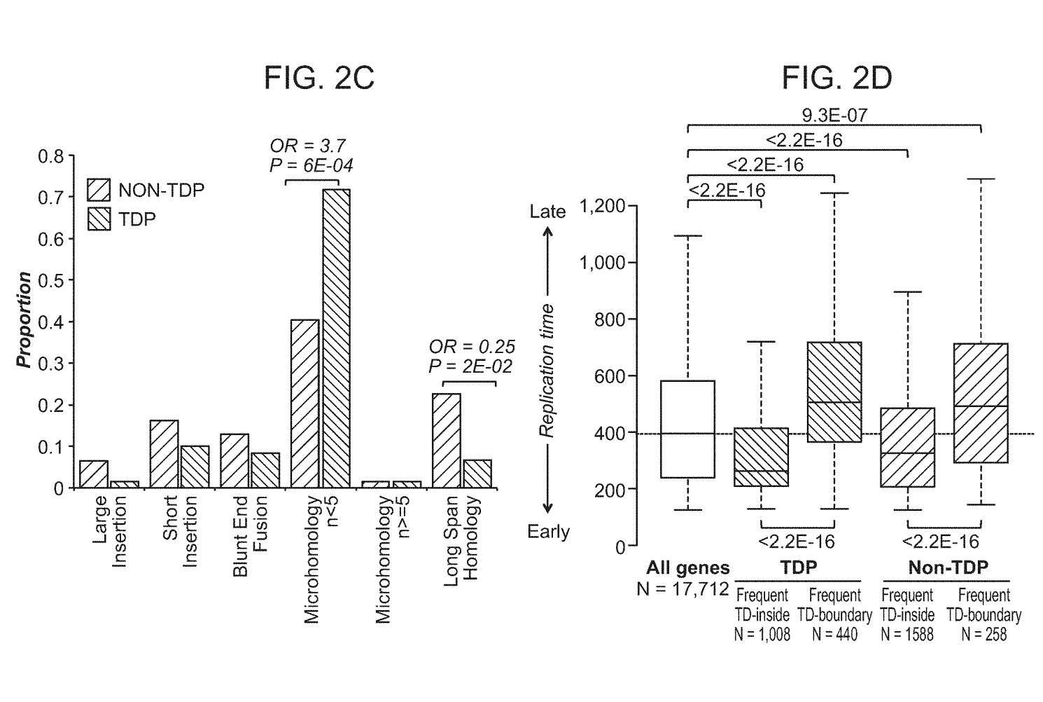

A comparison between the genomic properties of structural rearrangements occurring in TDP and non-TDP samples show a striking difference in the per-sample median TD span size, with TDP samples having significantly smaller median spans (median span size=89.9 Kb for TDPs and 1,189.7 Kb for non-TDPs, P=7.23E-09, FIG. 2A). More specifically, by plotting the distribution of the collection of all individual TD spans for TDP and non-TDP genomes (WGS dataset, n=50 and 227, respectively), it was observed that, whereas non-TDP tumors feature a continuum range of very large TDs reaching a plateau at around 1 Mb, TDP samples are characterized by two sharper TD span distribution modes, at ˜10 Kb and at ˜250 Kb (FIG. 2B). This suggests that in TDP tumors the mechanism for generating TDs may be different from that for non-TDP tumors.

Direct sequencing of the rearrangement junctions of 122 TDs from eleven different TNBC cell lines, both of TDP and non-TDP types, and analysis of the sequences at the breakpoint junctions revealed patterns indicative of specific DNA repair mechanisms. The validated breakpoint junctions were classified into those characterized by the presence of short (<10 bps) or long insertions, short (<5 bps), long or no microhomology; or long-range imperfect homology (FIG. 2C). The large majority of TDs in TDP tumors (72%, range 46-82%) show overlapping microhomology between the two DNA segments contributing to the rearrangement junction, which has been suggested as a signature of Non-Homologous End-Joining (NHEJ). Significantly, only 40% (range 27-86%) of TDs found in non-TDP tumors show a similar profile (OR=3.6, P=6E-04; FIG. 2C and data not shown). By contrast, TD rearrangements characterized by long-range imperfect homology, a signature indicative of non-allelic homologous repair (NAHR), are prevalent in non-TDP tumors (23%, range 0-50%, vs. 7%, range 0-31%, in TDPs; OR=0.25, P=2E-02; FIG. 2C and data not shown). While not wishing to be bound by any particular theory, these differences indicate that distinct DNA repair mechanisms, NHEJ vs. NAHR, may be operative in TDP and non-TDP tumors, respectively.

Recent evidence has revealed meaningful correlations between DNA replication timing, genomic instability and the emergence of DNA mutations. Indeed, a significant association was found between TD-affected genes and replication timing. Genes truncated by TD boundaries are found in late replication regions and genes spanned by TDs are enriched in early replicating regions (FIG. 2D). This specific pattern of replication timing is consistent across all samples (TDPs and non-TDPs), and it may reflect a shortage of DNA repair opportunities in late S phase, leading to an increase incidence of mis-repaired double strand breaks resulting in copy number variations. However, given that DNA replication typically encompasses ˜400-800 Kb chromosomal domains, it is plausible that the shorter TDs found in TDP genomes are generated within intra-replication timing domains, whereas, the larger, non-TDP TDs are more likely to result from the spatial proximity of distinct replication domains through the tridimensional looping of chromatin structures.

### Example 4 The TDP is Characterized by the Coordinated Perturbation of Several Cancer Genes

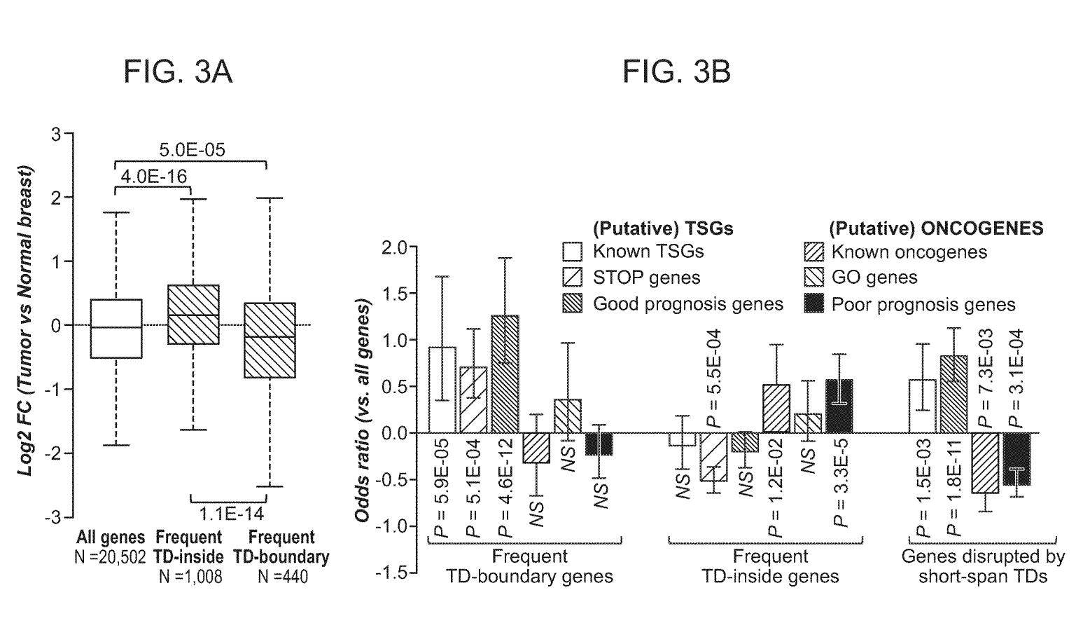

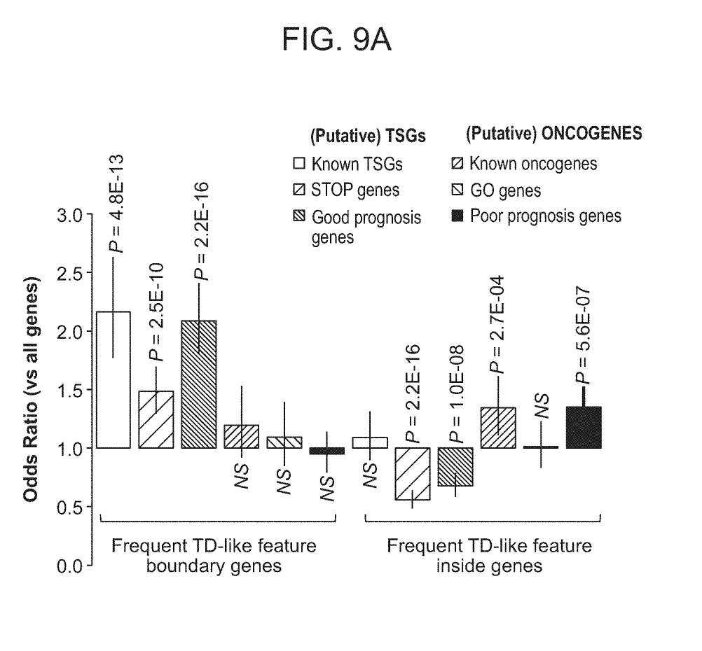

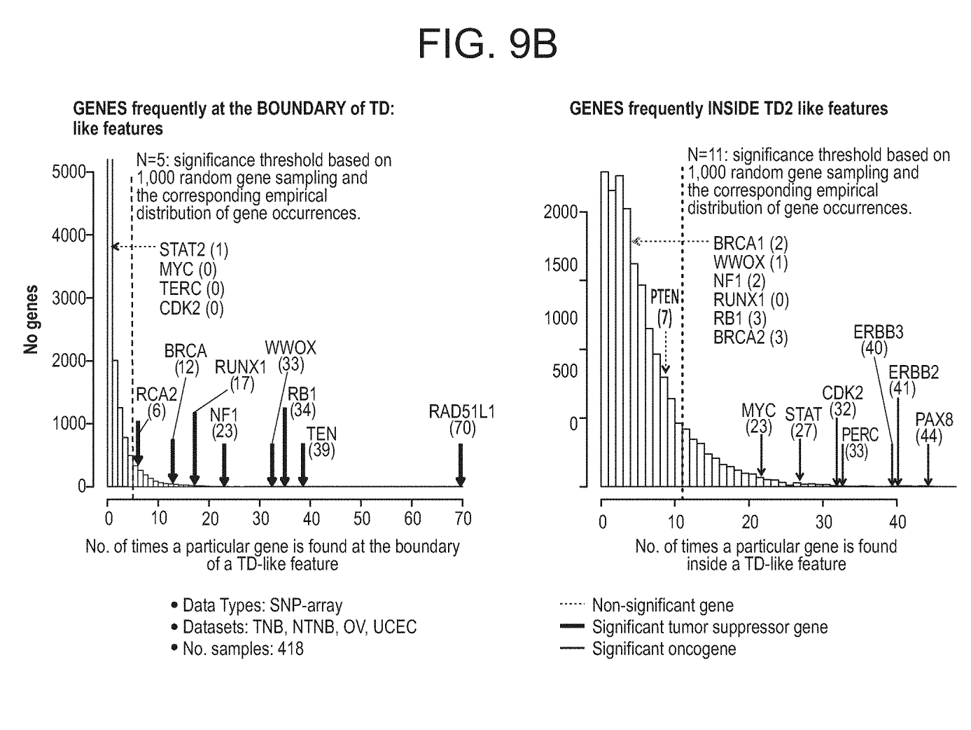

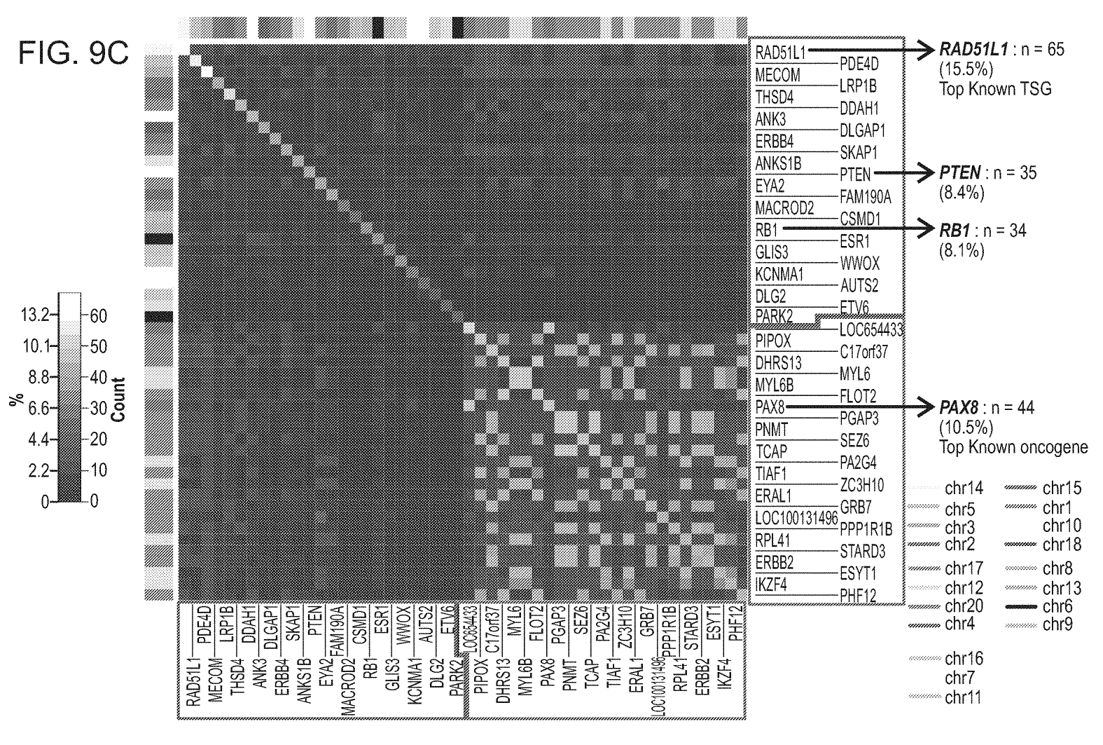

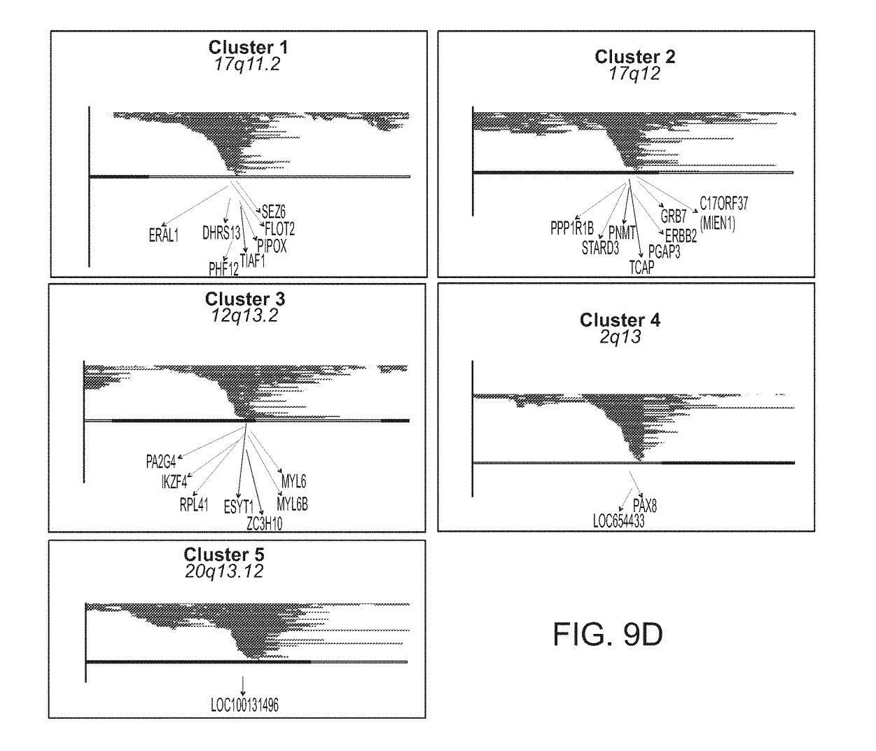

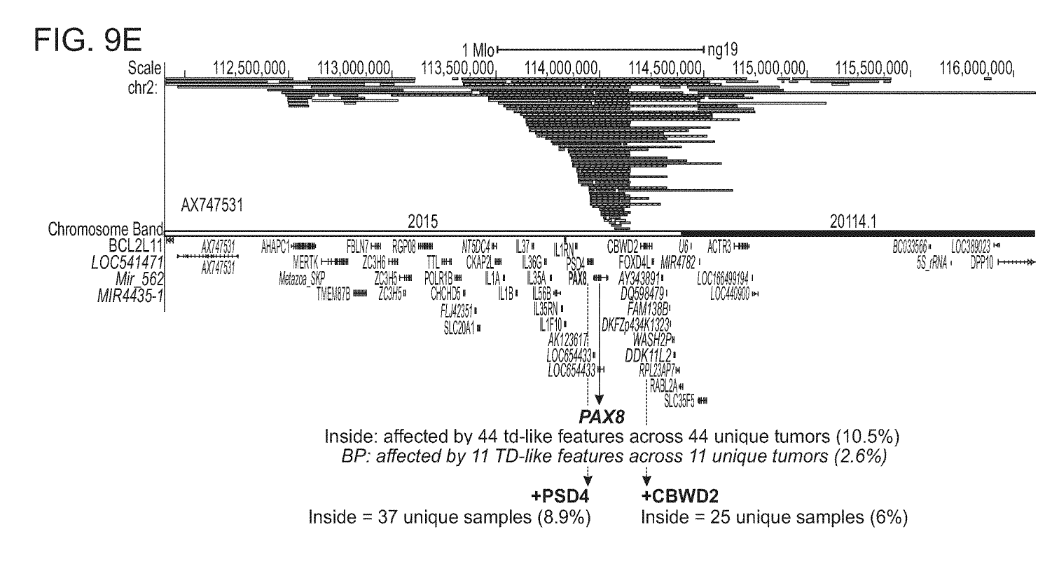

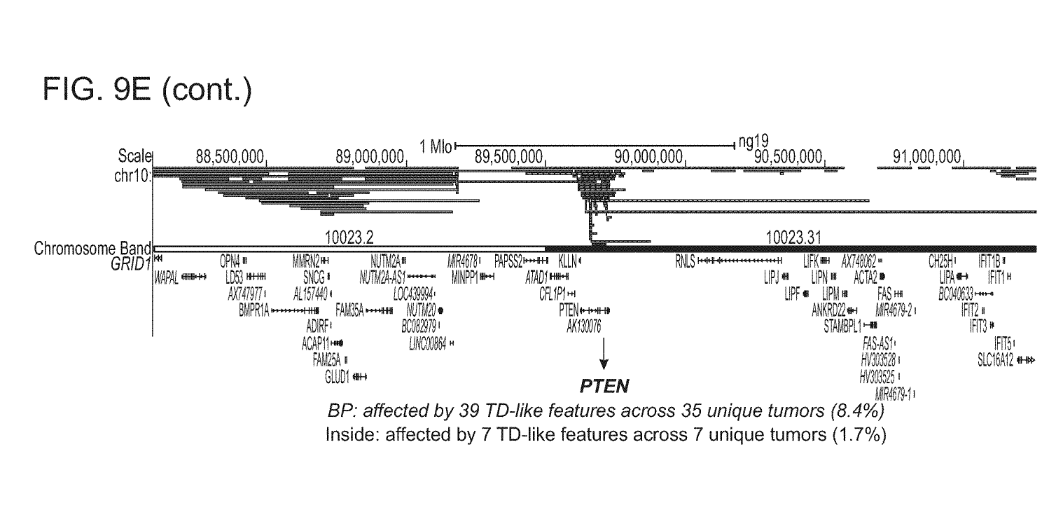

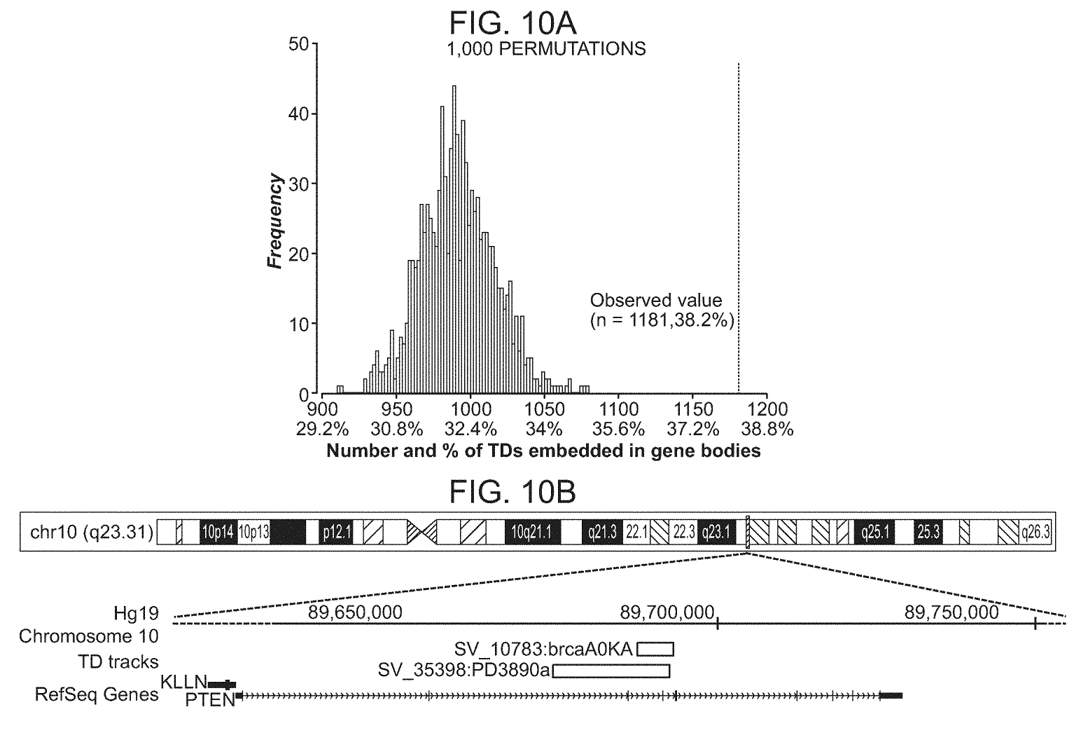

One of the most direct consequences of DNA segmental duplication is the increased expression of the genes that are entirely comprised within the rearrangement, whose copy number is thus augmented. It was hypothesized that a genomic configuration generating a large number of segmental duplications would represent a cancer genomic strategy for the modulation of hundreds of potential oncogenic signals, providing a selective advantage for the TDP cancer cell. To assess this possibility, changes in gene expression were compared between normal and tumor breast samples, with respect to the genes found to be most frequently affected by TDs in the TDP breast cancer WGS dataset (n=23, data not shown). As hypothesized, genes that are frequently found inside TDs are generally over-expressed in breast cancers when compared to the normal breast epithelium (median log₂ fold change=0.17, P=4.0E-16). In contrast, genes frequently located at the boundaries of TDs appear to be down-regulated in breast cancers (median log₂ fold change=−0.3, P=5.0E-05) (FIG. 3A). Moreover, genes frequently encompassed by TD segments are enriched in known oncogenes and genes whose increased expression levels associate with poor prognosis for breast cancer patients; whereas genes that map to TD boundaries are most significantly associated with known (P=5.9E-05) and putative tumor suppressors genes (STOP genes, P=5.1E-04; good prognosis gene, P=4.6E-12; FIG. 3B). These findings were confirmed by identifying the genes affected by TD-like features predicted using SNP-array data, which provided a significantly larger dataset (n=418 TDP tumor samples) (FIG. 9A). Indeed, well-known oncogenes such as PAX8, ERBB2 and MYC are among the most recurrent genes that are spanned by a TD across TDP samples, while known tumor suppressor genes such as RAD51L, PTEN, and RB1 populate the top list of genes affected by TD breakpoints (FIG. 9B and data not shown).

This systems strategy to generating the cancer state supposes that many different combinations of oncogenic signals would suffice as opposed to a single dominant oncogenic cassette such as that proposed for genes associated with ERBB2 amplification. To test this, the frequency of specific one-gene and multiple-gene combinations affected by tandem duplications was examined across 418 TDP genomes, assessed using SNP-array data (TNB, NTNB, OV and UCEC datasets), and it was found that only up to a maximum of 15.5% of tumors share TD-like features affecting a single common tumor suppressor gene (i.e., RAD51L1 and, at lower frequencies, WWOX, NF1, RB1, PTEN, BRCA1 FIG. 9B and data not shown), and even less frequently an oncogene (i.e., PAX8, duplicated in 10.5% of tumors, followed by ERBB2, ERBB3, TERC, STAT2, CDK2, and MYC, FIG. 9B and data not shown). The rest of the list of affected genes fall quickly in frequency. In addition, two gene combinations are relatively rare, with the top scoring gene pairs being those that map within a short distance of each other and are therefore affected by the same TDs (e.g., PAX8 and PSD4, coordinately duplicated in 8.9% of the tumors examined; or PAX8 and CBWD2 or IL1RN in 6%, FIGS. 9C, 9D and 9E). Much rarer are 2-gene combinations comprising frequent TD-boundary genes (FIG. 9C), arguing against the presence of a dominant TD-affected cancer gene or small gene set.

Intriguingly, it was observed that the shorter span TDs seen exclusively in TDP (˜10 kb) do not cause the segmental duplication of full-length genes but disrupt gene body integrity. It was found that 38.2% (1,181 out of 3,086) of the short span TDs (span<100 kb) present in the 50 TDP cancer genomes analyzed by WGS, are completely embedded within a gene body often disrupting the intron/exon structure (P<0.001, FIG. 10). Moreover, it was observed that the genes affected by these short TDs are more likely to function as anti-cancer as opposed to pro-cancer genes, as they are enriched in TSGs and putative TSGs, while being depleted for oncogenes (FIG. 3B).

Taken together, these results strongly suggest that the consequence of generating many tandem duplications is a systems mechanism to moderately augment the expression of many oncogenes and suppressing the expression of anti-oncogenes/tumor suppressors simultaneously. In this model, there is no obvious genetic driver by virtue of levels of expression or the frequency of occurrence. Given these findings, and the fact that the TDP characteristic should have been established before the generation of TDs, Applicant sought genetic signals that would distinguish TDP from non-TDP that are not found in the genes directly affected by TDs and that would appear in higher frequencies in the TDP.

### Example 5 Insights into the Molecular Background Favoring TDP Formation

In the first analysis, enrichment of specific tandem duplications was not found in the TDP tumors that could explain the unique genomic features associated with the phenotype. This result suggested that there may be intrinsic molecular differences between TDP and non-TDP tumors that induce the TDP and that the changes in gene expression arising from tandem duplicons are a consequence of the TDP.

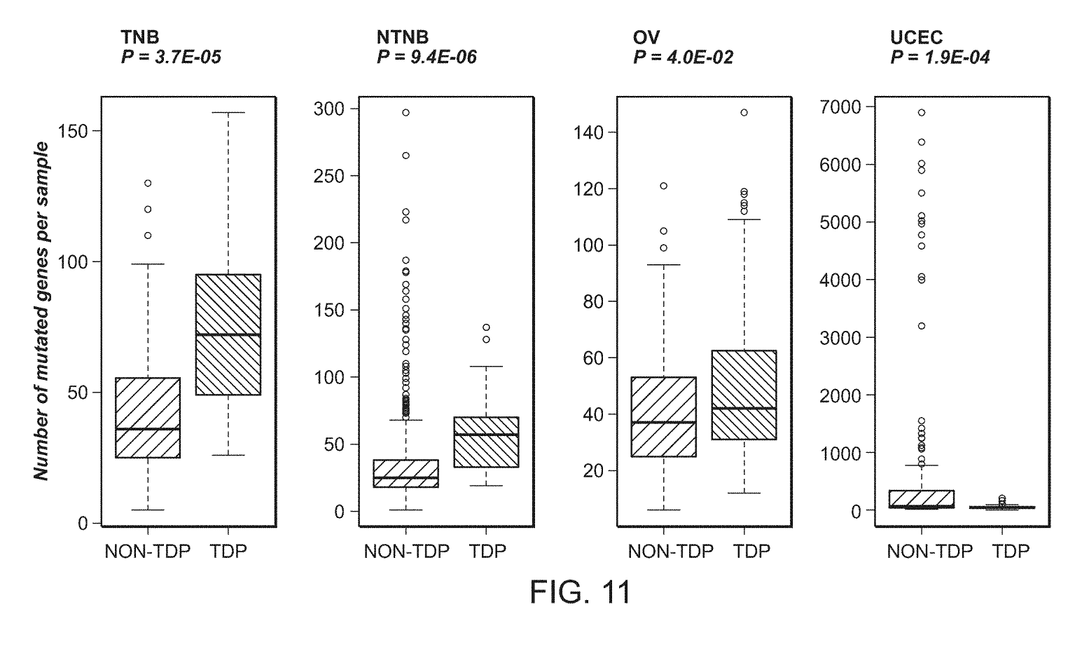

To identify factors that may correlate with the molecular mechanisms underlying the phenotype, the characteristics of TDP as compared to non-TDP samples was investigated within each of the three most highly TDP-enriched tumor data sets: TNB, OV and UCEC. In addition, analysis was extended to non-TNBCs (NTNB), which, although depleted in TDPs as a cancer group, comprised a sufficient number of TDP and non-TDP samples to perform statistical comparisons. First, the overall mutation burden was computed, as the total number of genes per sample that are affected by at least one non-silent mutation as assessed by exome sequencing. Although the TNB, the NTNB and, to a lesser extent, the OV datasets showed a significant higher mutation burden in the TDP subgroup, this trend was not consistent in the remaining dataset (UCEC, FIG. 11).

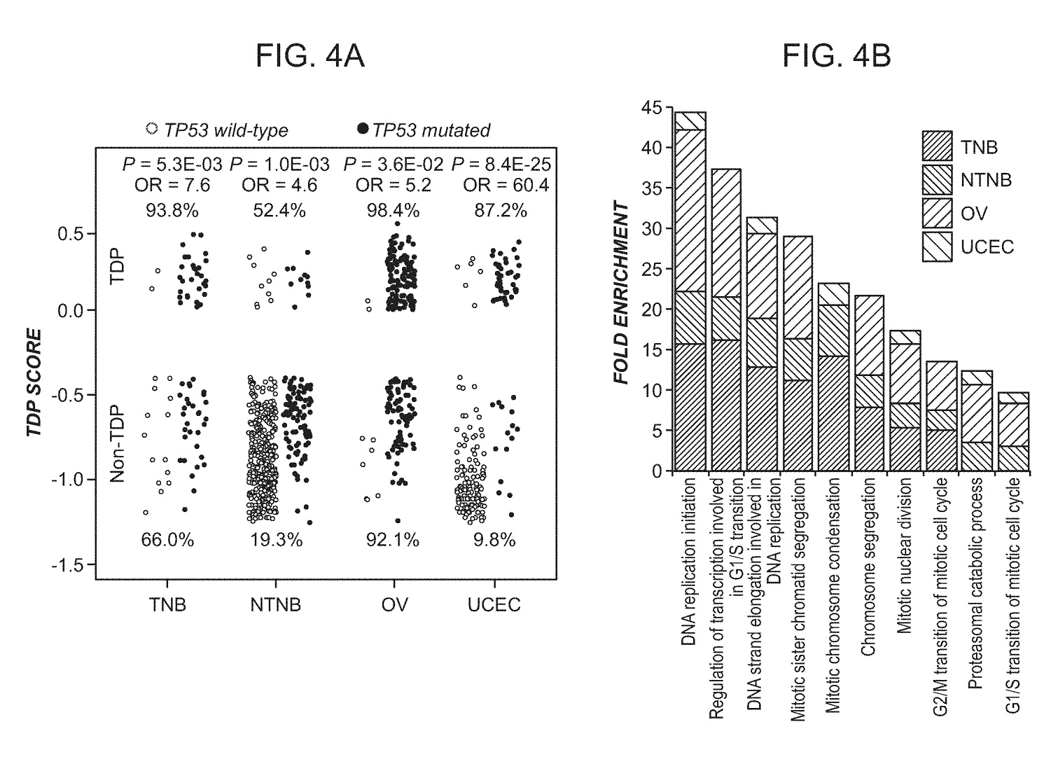

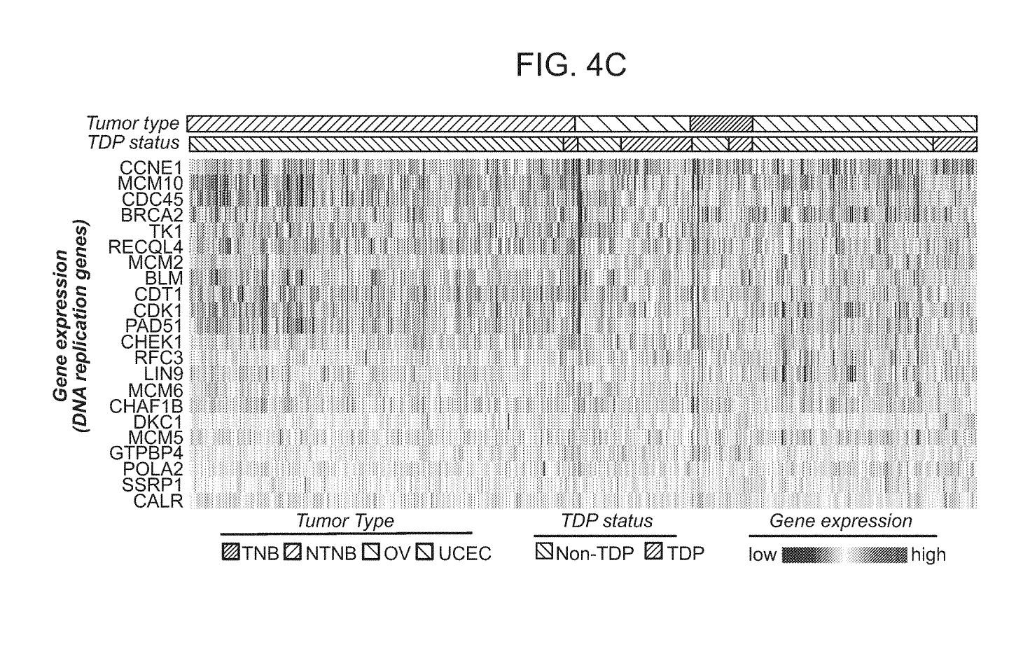

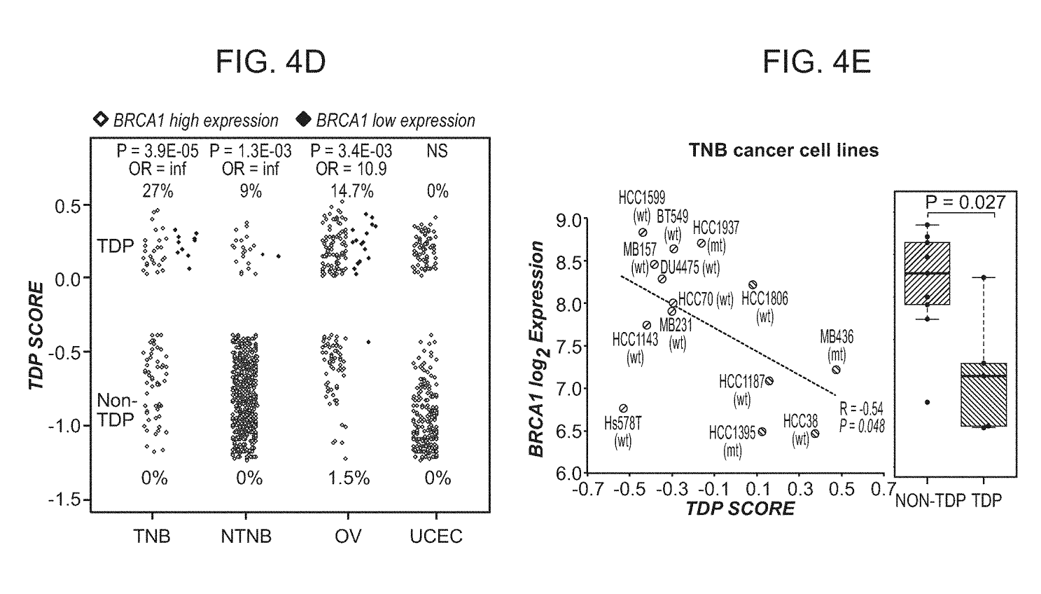

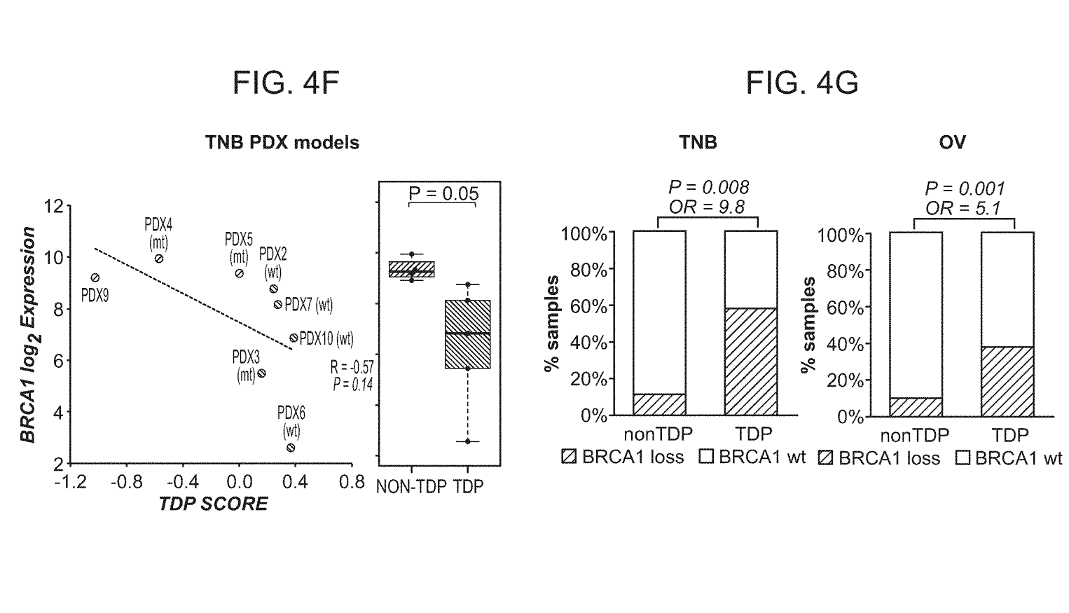

Therefore, efforts were focused on individual gene mutations to search for genes that, when mutated, are associated with the TDP. For each cancer dataset analyzed, a list of frequently mutated genes (i.e. mutated in at least 15% of cases within either the TDP or the non-TDP sample subgroups) was compiled. Mutation frequencies were then compared between TDP and non-TDP tumors using the Fisher's exact test and significant differences were assessed across cancer datasets (data not shown). Of a total of 56 frequently mutated genes, the TP53 gene is the only one whose somatic mutation rate is recurrently higher in TDP relative to non-TDP samples across different tumor types, with all of the four examined datasets showing a significant enrichment (TNB, odds ratio (OR)=7.6; NTNB, OR=4.6; OV, OR=5.2; UCEC, OR=60.4) (FIG. 4A and data not shown).

It was then asked whether TDP and non-TDP tumors show profiles of differential gene expression that distinguish these two states. Following the identification of differentially expressed genes (DEGs) between TDP and non-TDP tumors within each tumor-type dataset, a Gene Ontology enrichment analysis was performed for the lists of up- and down-DEGs, to identify biological processes most commonly perturbed in association with the TDP. Up-regulation of genes engaged in biological processes relevant to cell proliferation and DNA replication appeared to be the most robustly and consistently enriched across all four analyzed datasets (FIG. 4B). This strongly suggests that TDPs are more prone to increased/perturbed DNA replication and faster cell growth. Among the DNA replication genes most frequently up-regulated (in at least three out of the four datasets examined), CONE1 was the one with the highest cumulative fold change, followed by several critical DNA replication initiation factors, including CDT1, MCM2, MCM6 and MCM10 (FIG. 4C and data not shown).

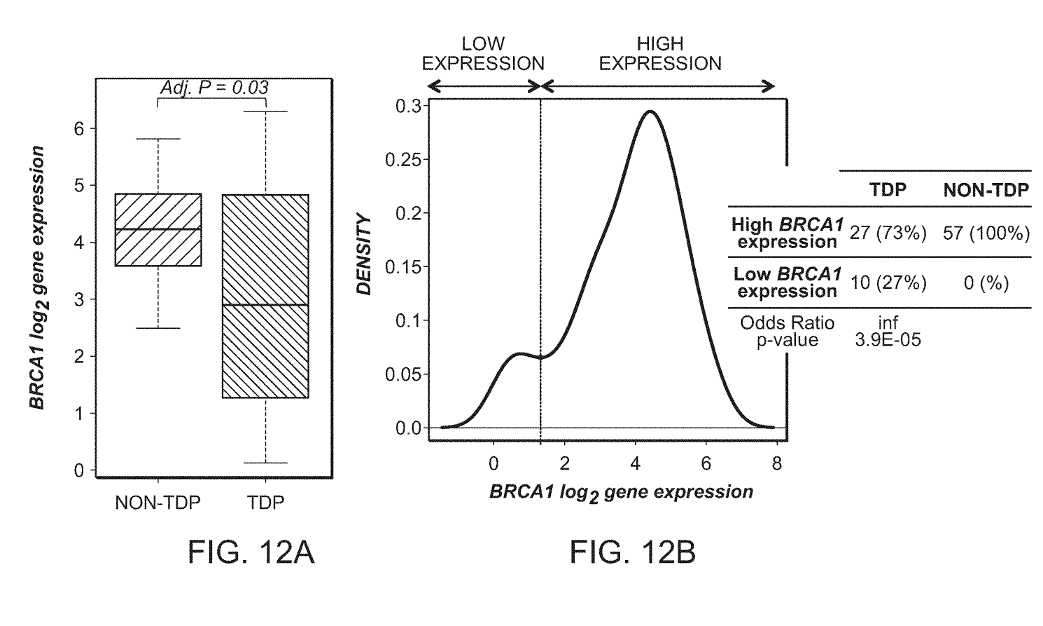

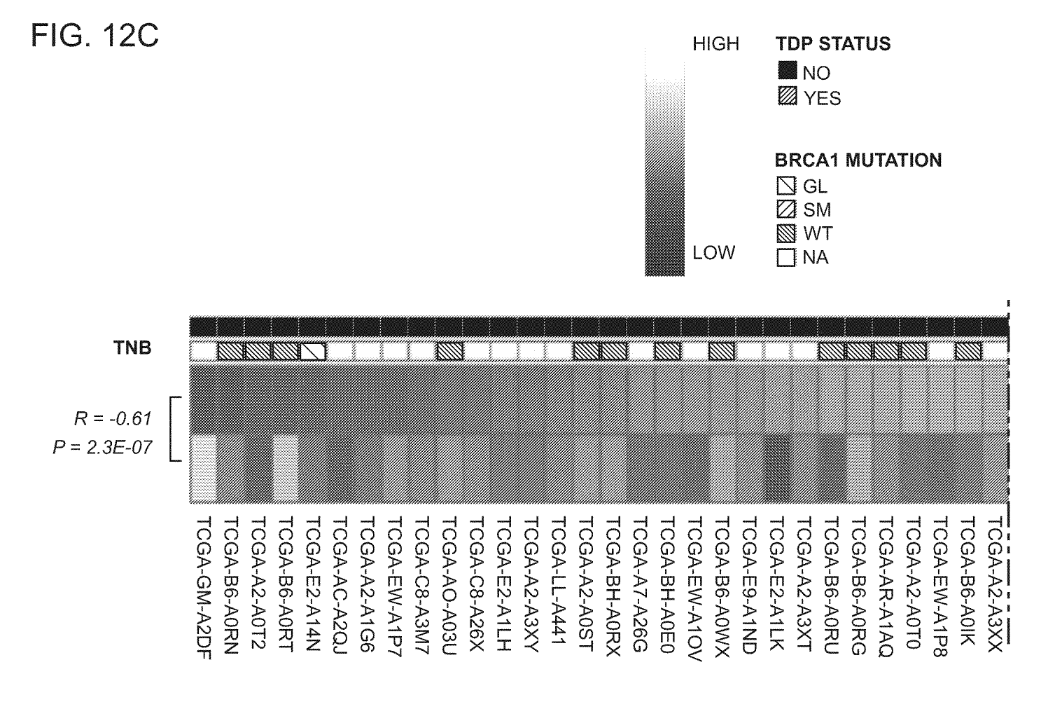

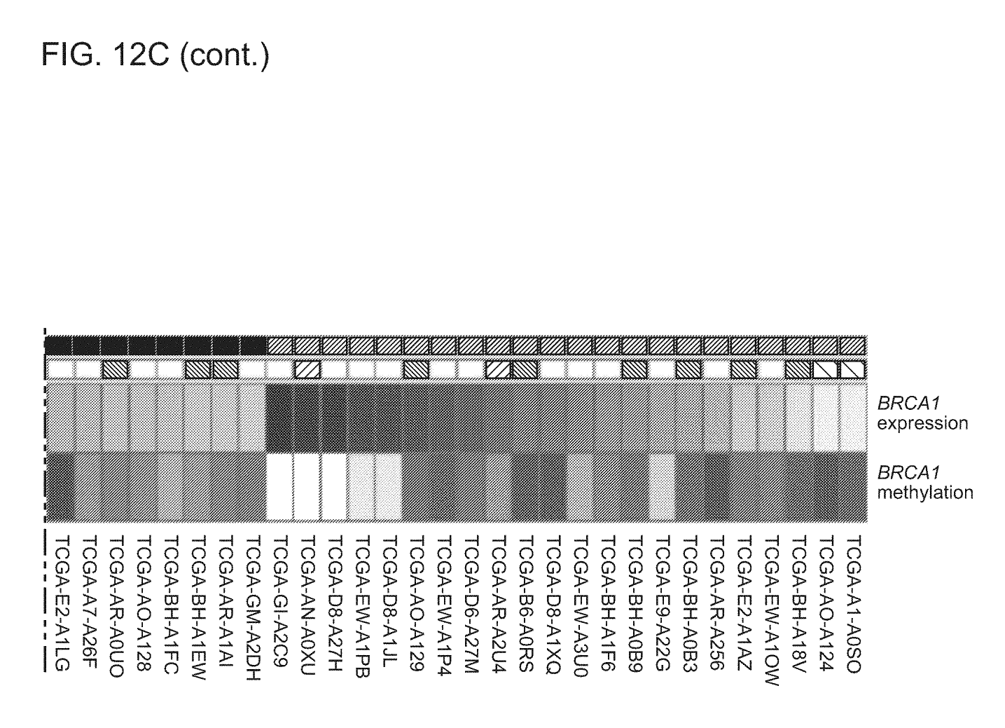

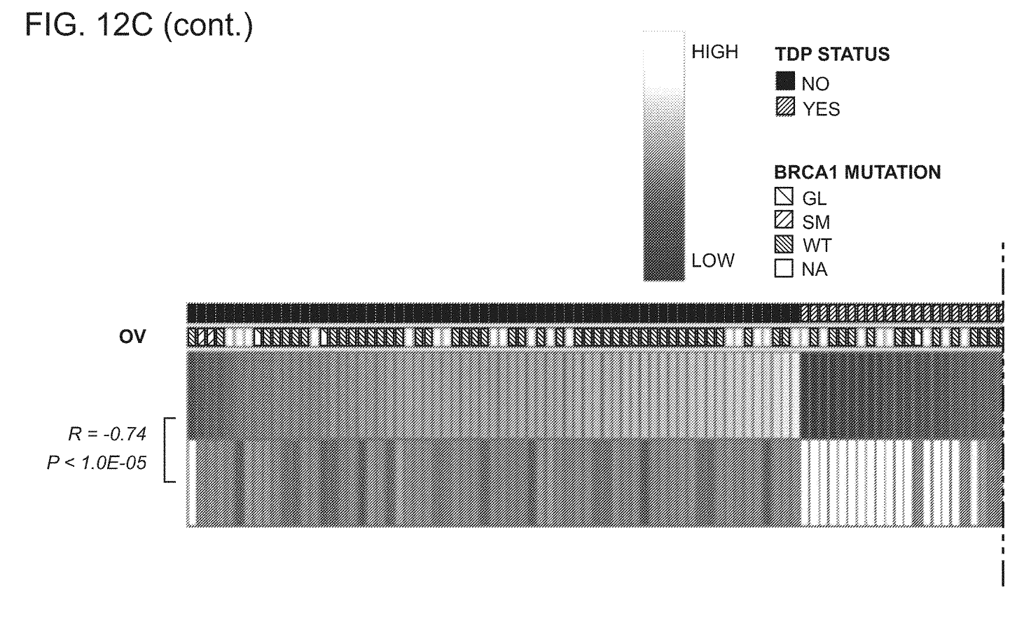

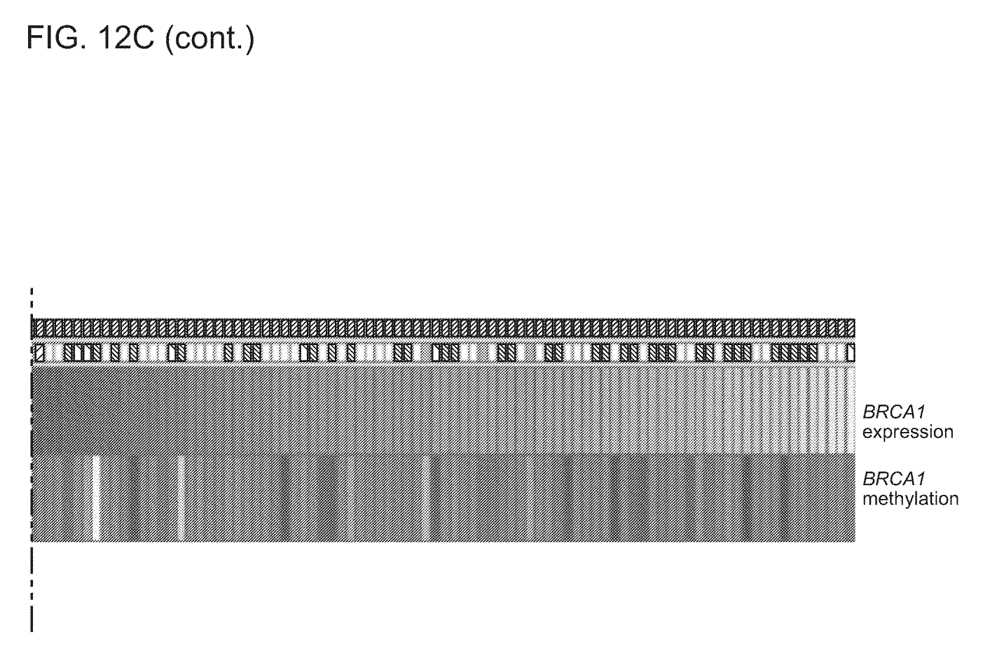

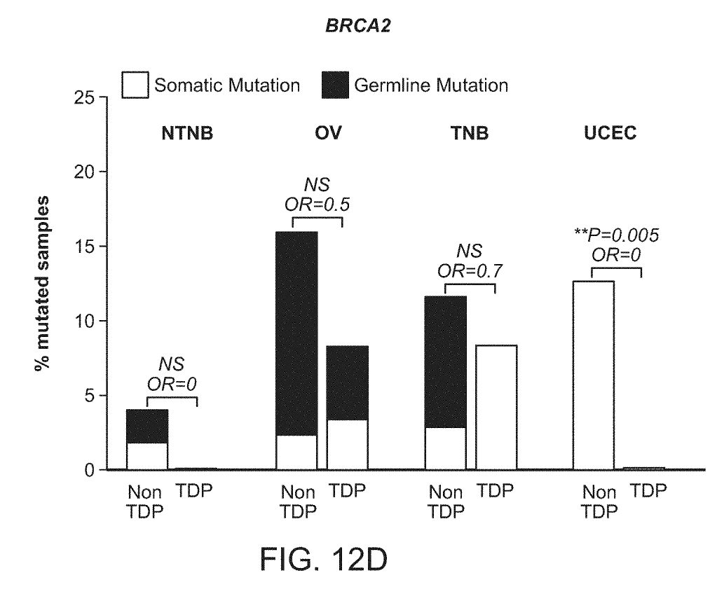

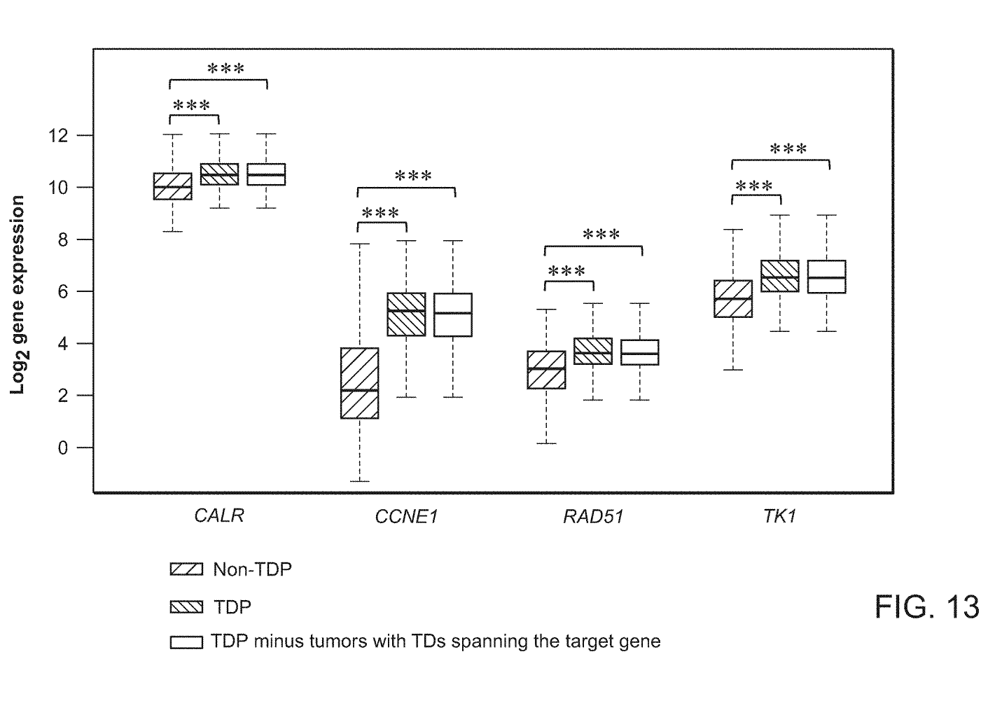

Though no multigene cassettes engaged in specific biological process appeared to be consistently down-modulated in the TDP datasets (data not shown), it was observed in the cancer subgroup of TNBC that the BRCA1 gene is among the most significantly down-regulated genes, with a greater than two-fold decrease in TDP versus non-TDP tumors (P=0.03; FIGS. 12A and 12B). Indeed, a highly significant enrichment for TDP tumors was found in BRCA1 low-expressors (27% of all TDP samples compared with 0% of non-TDP TNBC samples, P=3.9E-05). The strong association was validated between low BRCA1 expression and the TDP score in the NTNB (P=1.3E-03) and OV (P=3.4E-03) datasets (FIG. 4D), and in two other independent TNBC datasets (P=0.027 and P=0.05), all showing an overall negative correlation between BRCA1 expression level and TDP score (FIGS. 4E & 4F). There did not appear, however, to be any enrichment in BRCA1 sequence mutations that distinguishes TDP. Given the critical involvement of BRCA1 in DNA repair by homologous recombination and the paucity of NAHR at the tandem duplication breakpoints, its reduced expression raises the possibility of a role in the development of the TDP.

Furthermore, a significant association was found between BRCA1 promoter methylation status and reduced BRCA1 expression levels in the TNB (R=−0.61, P=2.3E-07) and OV (R=−0.74, P<1.0E-05E) datasets (FIG. 12C), pointing at epigenetic silencing as a key mechanism of transcriptional inactivation of BRCA1 in TDP tumors.

Whereas no enrichment in BRCA1 somatic mutations was found that distinguishes TDP, when somatic and germline mutations and promoter hypermethylation were combined, a significant increase in the frequency of BRCA1 disruption in TDP vs. non-TDP tumors was found in the TNB and OV datasets (OR=9.8 and OR=5.1, P=8.0E-03 and P=1.0E-03, respectively; FIG. 4G). On the contrary, BRCA2 mutation rates did not show any association with the TDP and, instead, appeared to be modestly but consistently higher in the non-TDP tumor sets (FIG. 12D), raising the hypothesis that the TDP is an exquisite feature of BRCA1 loss and not of BRCA2 loss.

When taken together, these results suggest that a combination of TP53 loss of function mutation, BRCA1 reduced expression and overexpression of DNA replication and cell cycle genes may be required for TDP generation.

The data established that certain multigene expression changes are strongly associated with the TDP phenotype. It was investigated whether changes were a result of the tandem duplications or preceded the induction of these structural mutations. Of the 23 differentially expressed genes involved in DNA replication and Cell Cycle associated with TDP, only four (i.e., CALR, CCNE1, RAD51, TK1) were also found inside TDs in multiple TDP samples but at modest frequencies of <5% (data not shown). After removing the TDP tumor samples harboring physical TDs spanning these four differentially expressed DNA replication and cell cycle genes, the association of these genes with the TDP remains statistically significant (FIG. 12). This suggests that their overexpression is likely to be engaged in the establishment of the TDP and not simply a consequence of the phenotype.

### Example 6 The TDP as a Genomic Marker for Drug Sensitivity

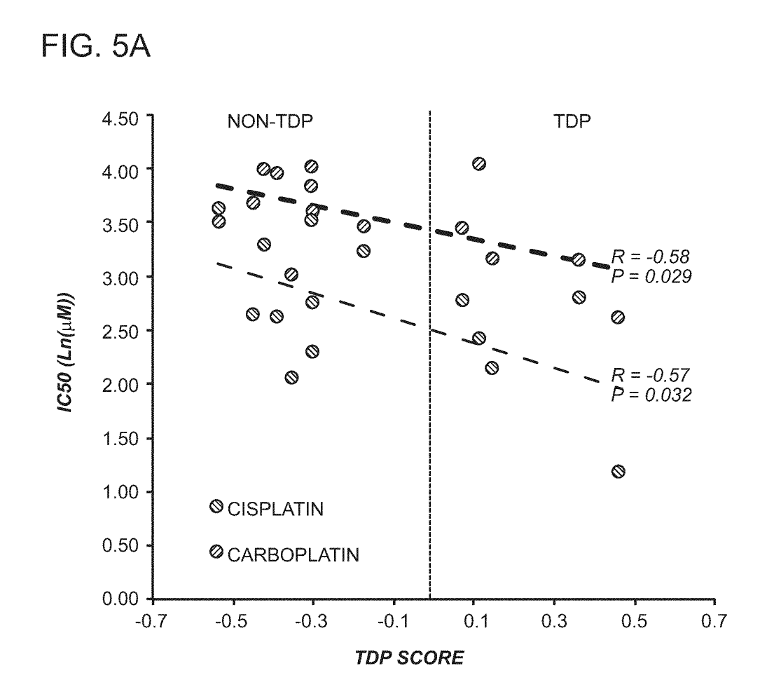

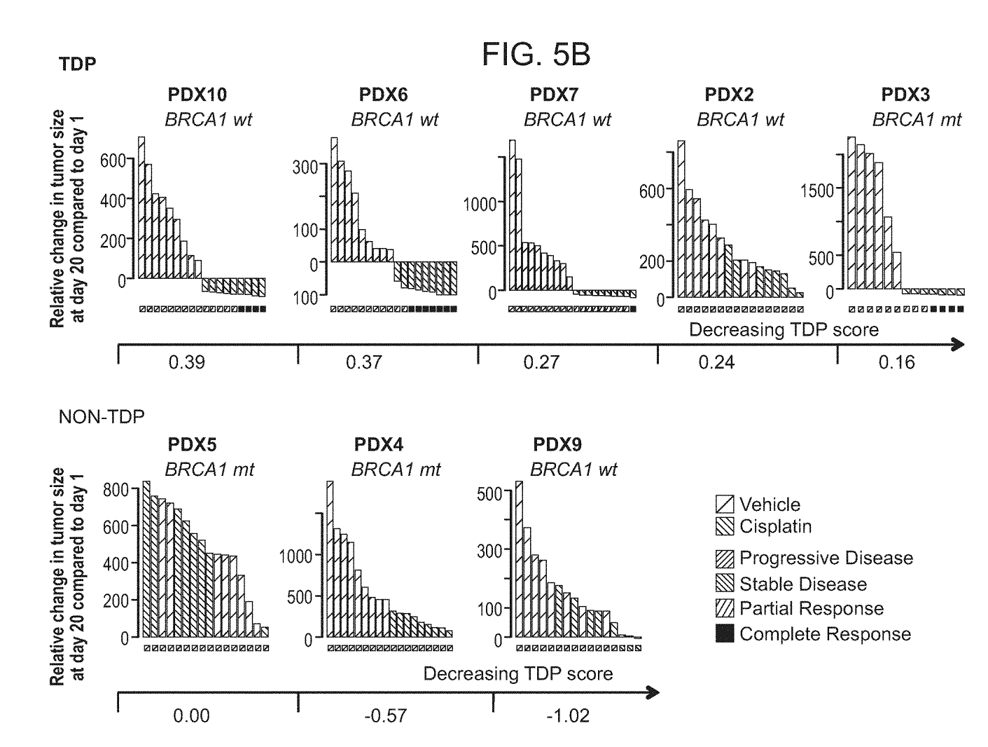

The TDP could represent a marker for drug sensitivity by searching the Genomics of Drug Sensitivity in Cancer (GDSC) database for drugs and compounds which differed in their effect between the TDP and non-TDP breast cancer cell lines. Interestingly, cisplatin was among six drugs showing a significant positive correlation between TDP scores (computed based on available WGS data) and IC₅₀ values (Table 2). Given the utility of platinum-based therapeutics as neoadjuvants in the clinical management of TNBC patients, and the reported association between platinum-based treatment clinical success and a BRCAness molecular profile, it was hypothesized that the TDP subset of TNBCs may be characterized by a better response to platinum-based chemotherapy. Thus, a total of 14 genomically characterized TNBC cell lines were tested, and significant negative correlations between IC₅₀ values relative to both cisplatin and carboplatin treatments and the TDP score (R=−0.57, P=0.032 for cisplatin; R=−0.58, P=0.029 for carboplatin) were found (FIG. 5A). By contrast, olaparib, a PARP-inhibitor shown to have anti-tumor activity in BRCA-mutated cancer patients, did not show any significant association with the TDP score (Table 3), suggesting that the sensitivity of TDP tumors to cisplatin may not be exclusively related to the mutational status of BRCA1 or BRCA2.

TABLE 2
Compounds showing increased sensitivity in TDP breast cancer cell lines
based on data from the Genomics of Drug Sensitivity in Cancer data base

| | Drug | Target | TDP | | | | | |
|---|---|---|---|---|---|---|---|---|
| Cell Line | | | MCF-7 | HCC38 | HCC1187 | HCC1395 | HCC2157 | |
| Subtype | | | ER/PR+ | TNBC | TNBC | TNBC | TNBC | |
| TDP Score | | | 0.385 | 0.367 | 0.152 | 0.118 | 0.076 | |
| IC₅₀ Ln | NVP- | ALK | na | na | 0.62 | na | 0.38 | |
| (μM) | TAE684 | | | | | | | |
| | BMS- | IGF-1R | na | na | 2.81 | na | 3.64 | |
| | 536924 | | | | | | | |
| | Cytarabine | DNA | 0.69 | 0.63 | 3.36 | 1.1 | na | |
| | | Synthesis | | | | | | |
| | Cisplatin | DNA | 2.72 | 5.07 | 4.76 | 4.96 | na | |
| | | Crosslinker | | | | | | |
| | Tipifarnib | FNTA | −1.79 | 1.81 | 2.03 | 2.7 | −1.13 | |
| | | (Ras) | | | | | | |
| | Midostaurin | KIT | −1.1 | −0.7 | 2.07 | −2.47 | 0.55 | |
| non-TDP | | | | | | | R | P-value |
| HCC1937 | T47D | MB231 | HCC1143 | HCC1954 | HCC1599 | HCC2218 | | |
| TNBC | ER/PR⁺ | TNBC | TNBC | ERBB2⁺ | TNBC | ERBB2⁺ | | |
| −0.17 | −0.269 | −0.306 | −0.424 | −0.434 | −0.447 | −0.849 | | |
| na | na | na | na | na | 2.98 | 5.37 | −0.99 | 0.009 |
| na | na | na | na | na | 5.27 | 6.22 | −0.98 | 0.017 |
| 3.32 | 3.47 | 2.18 | na | 3.41 | na | 4.68 | −0.84 | 0.005 |
| 4.92 | 4.86 | 5.57 | na | 6.94 | na | 5.99 | −0.71 | 0.032 |
| 2.2 | 3.85 | 1.76 | 2.59 | 2.66 | 5.23 | 4.32 | −0.70 | 0.012 |
| 3.2 | 1.66 | −1.27 | 3.73 | 3.62 | −0.13 | 4.42 | −0.64 | 0.026 |

TABLE 3
Correlations between cisplatin or carboplatin sensitivities
and TDP score/BRCA1 expression level in TNBC cell lines

| | | BRCA1 | Cisplatin | Carboplatin | Olaparib |
|---|---|---|---|---|---|
| | TDP | Exp. | IC₅₀ Ln | IC₅₀ Ln | IC₅₀ Ln |
| Cell Line | Score | (log₂) | (μM) | (μM) | (μM) |
| MB436 | 0.469 | 7.22 | 1.17 | 2.61 | 3.77 |
| HCC38 | 0.367 | 6.46 | 2.8 | 3.15 | 1.99 |
| HCC1187 | 0.152 | 7.07 | 2.15 | 3.17 | 3.57 |
| HCC1395 | 0.118 | 6.48 | 2.4 | 4.04 | 4.28 |
| HCC1806 | 0.08 | 8.23 | 2.76 | 3.45 | 2.40 |
| HCC1937 | −0.17 | 8.71 | 3.22 | 3.46 | 4.51 |
| BT549 | −0.3 | 8.64 | 2.28 | 4.01 | 3.09 |
| HCC70 | −0.3 | 8 | 2.75 | 3.61 | 4.19 |
| MB231 | −0.306 | 7.91 | 3.52 | 3.83 | 3.49 |
| DU4475 | −0.36 | 8.28 | 2.04 | 3.01 | na |
| MB157 | −0.39 | 8.47 | 2.62 | 3.96 | 3.16 |
| HCC1143 | −0.424 | 7.74 | 3.3 | 4 | 4.57 |
| HCC1599 | −0.447 | 8.85 | 2.65 | 3.68 | na |
| Hs578T | −0.54 | 6.76 | 3.62 | 3.51 | 4.61 |
| R/P-value | | | −0.57/0.032 | −0.58/0.029 | −0.45/0.14 |
| vs. | | | | | |
| TDP Score | | | | | |
| R/P-value | | | 0.05/0.39 | 0.25/0.07 | −0.02/1.00 |
| vs. | | | | | |
| BRCA1 Exp. | | | | | |

Remarkably, although the levels of BRCA1 expression correlate with the TDP score in the TNBC cell lines examined, significant association between BRCA1 levels and either cisplatin nor carboplatin IC₅₀ values (Table 3) was not observed, indicating that platinum sensitivity correlates more directly with the TDP score than with BRCA1 expression levels. This suggests that the TDP score, which is modulated by genes other than BRCA1, may be the key genomic predictor of cisplatin sensitivity in TNBC.

This hypothesis was explored further by testing in vivo response to cisplatin treatment in eight independent patient derived xenograft (PDX) models of TNBC. Following a three-week long cisplatin regimen, four out of the five TDP PDX models showed a remarkable partial response, with >80% tumor shrinkage of individual tumors, and in all five models cisplatin treatment caused a significant reduction in tumor growth when compared to the vehicle arm (FIG. 5B). On the contrary, none of the three non-TDP models analyzed exhibited a reduction in tumor volume after three weeks of treatment, and only one out of the three models showed a significant response to cisplatin when compared to the vehicle arm (FIG. 5B). Thus in both established cell lines and in in vivo patient derived xenografts, TDP status is strongly associated with cisplatin sensitivity.

The same eight PDX models were also tested for their sensitivity to doxorubicin and docetaxel, and it was found that the TDP was not associated with sensitivity to these other chemotherapeutic agents (data not shown). This suggests that TDP was uniquely associated with sensitivity to platinum-based chemotherapeutic agents such as cisplatin.

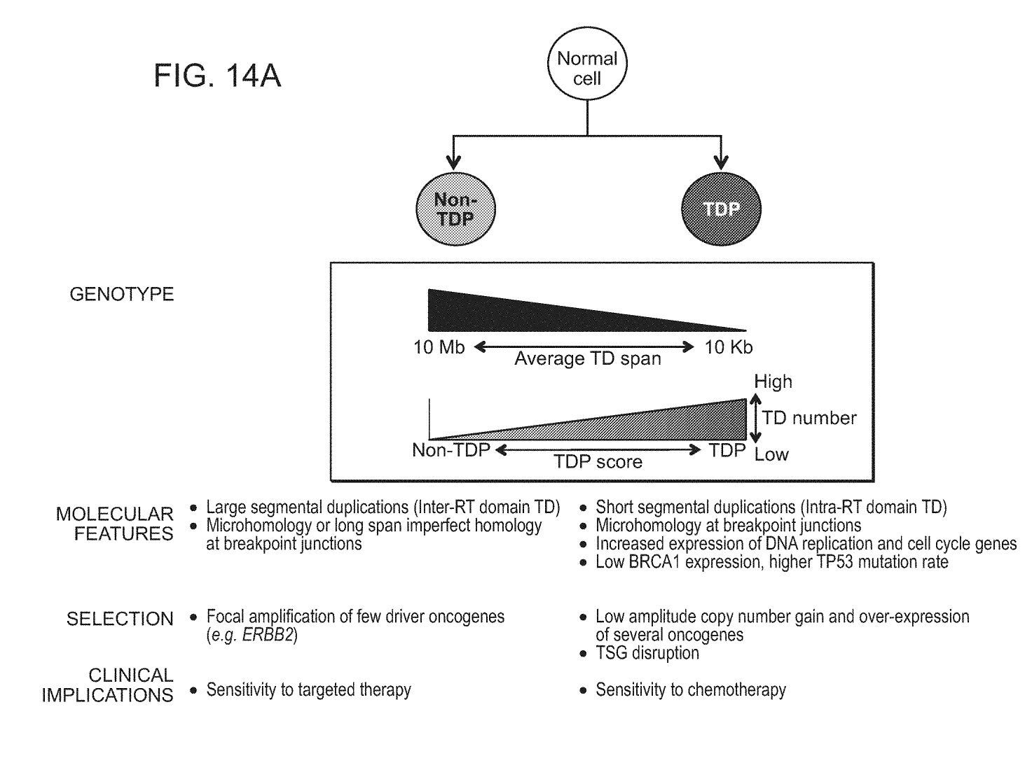

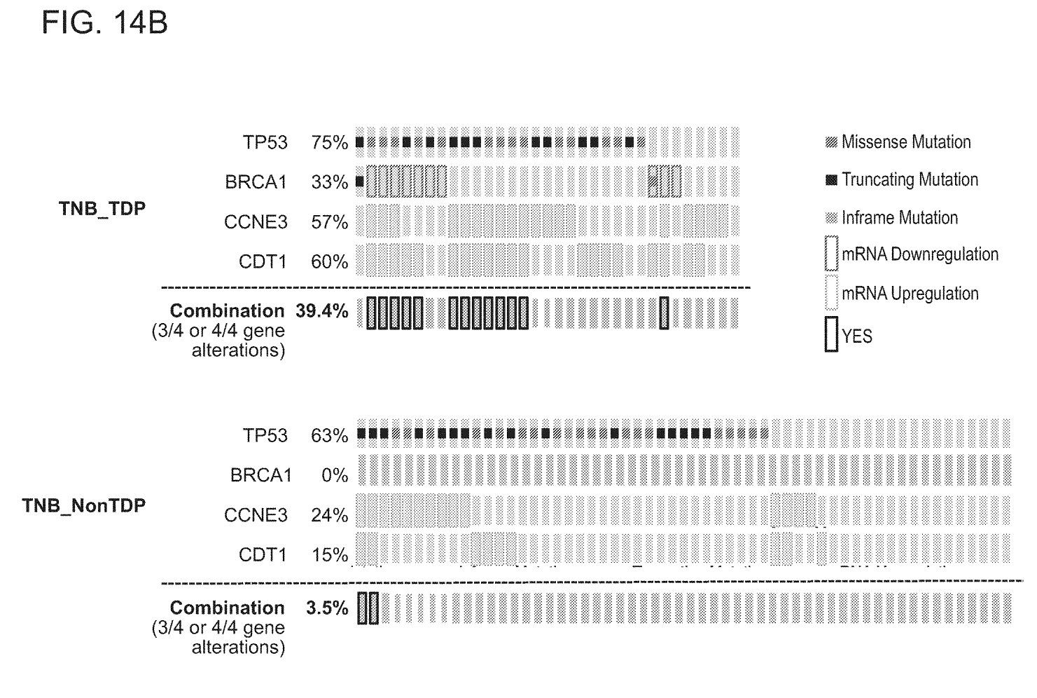

Recent studies have described previously unrecognized massive structural aberration events occurring on a genome-wide scale in human cancer. A fundamental challenge is to define a quantitative metric to systematically identify these global genomic configurations in cancer samples and to investigate the role they play in tumorigenesis. Here, the invention described herein provides an approach to the unbiased recognition of the Tandem Duplicator Phenotype (TDP). By applying this TDP scoring metric to a collection of ˜3,000 tumors with genomic data (WGS and/or SNP-array), statistical evidence was provided to show that the TDP is enriched in specific tumor types, suggesting a distinct biological mechanism underlying this phenotype that cuts across histological subtypes (FIG. 14A).

While not wishing to be bound by any particular theory, Applicant believes that DNA re-replication, such as MH-mediated break-induced replication (MMBIR) is a plausible mechanistic explanation for TD generation in the TDP chromotype.

Whereas cancers with high amplification of a single locus in non-TDP tumors depend on a dominant driver oncogene such as ERBB2 or MYC, the TDP is unusual in that there does not appear to be a discernible single cancer driver gene targeted by the TDP. Rather, different combinations of many potential drivers appear to be affected by the widespread genomic distribution of TDs. Indeed, in analysis of genes perturbed by tandem duplications in TDP, individual gene that appears to be affected in more than 15.5% of the samples examined was not found, and the level of overexpression and copy number changes are only modest by comparison (data not shown). However, the TDP configuration generates changes that affect the expression and function of hundreds of genes in a distributed manner within each tumor. Thus, TDP tumors take advantage of a systems strategy that generates genome-wide segmental tandem duplications to target the optimal expression or suppression of many cancer genes distributed across the genome. In seeking to uncover the root genetic aberrations that may underlie the induction of the TDP, gene expression and mutational profiles were examined, which are frequently found and most strongly associated with the TDP across a number of tumor types. The findings suggest that the TDP is induced by specific combinations of gene perturbations that (i) cause the loss of genome integrity (i.e., loss of TP53 and BRCA1) and (ii) drive the augmented expression of cell cycle and DNA replication genes (e.g., increased activity of CONE1, CDT1). In fact, combinations of these TDP-associated gene perturbations occur remarkably more frequently in TDP than in non-TDP TNB tumors (OR=17.2, P-value=2.1E-05, FIG. 14B). Earlier reports have suggested a BRCA1-independent mechanism for the TDP based on the absence of BRCA1 mutations in samples (breast and ovarian carcinomas) with a large number of TDs. The study here instead finds a strong negative correlation between BRCA1 gene expression and the TDP score, as well as the enrichment for BRCA1-defective tumors (assessed by the presence of somatic or germ line mutations, or promoter hypermethylation) in TNBC and ovarian carcinoma (FIGS. 4D-G). This strongly supports a previously unrecognized critical role for BRCA1 loss of function in the induction of the TDP.

Finally, quantitative assessment of the TDP may have clinical relevance. Here, an association between the extent of TDP and greater sensitivity to platinum-based chemotherapy was found, both in cell lines and in PDXs. It has been reported that breast tumors with perturbations of BRCA1 respond better to cisplatin treatment. Though our observations in vitro suggest that cisplatin sensitivity is better correlated with the TDP score than BRCA1 levels or mutational status, the TDP score integrates multiple genetic factors, such as TP53 status and select driver gene expression (e.g., CDT1 and CONE1), which may be the genetic components needed for the sensitivity phenotype. Whereas, recent neoadjuvant studies suggest that the effectiveness of cisplatin in TNBC is associated with loss of BRCA1 by mutation or low expression, data herein suggests that the BRCA1 status may be only a surrogate biomarker for the more important TDP, and that the TDP score is a more robust predictor of response to platinum-based chemotherapies independent of tumor type. Indeed, high TDP scores are enriched in TNBC, ovarian carcinoma and in the recently described cluster 4 endometrial carcinoma, which have been shown to share a similar transcriptional and molecular profile.

Given the specific molecular determinants associated with the TDP across tumor types, there is a benefit of a cisplatin and PARPi combination in treating TDP tumors.

Thus, the TDP assessment provided herein provides a unique genome-sequence-based predictive marker for platinum-based drug sensitivity, and allow for detailed interrogation of more precise mechanisms of cisplatin sensitivity.

### Example 7 Additional TDP Tumors

In this study, we have evaluated a large pan-cancer cohort comprising more than 2,700 independent tumor samples. Using our method of determining TD and TDP score as described herein, we discovered five additional tumor types that recurrently manifested the TDP configuration (i.e., TDP Score above zero). The five additional tumor types include: adrenocortical, esophageal, stomach adeno-, lung squamous cell and pancreatic adeno-carcinomas. See Table 4 below.

TABLE 4
Additional TDP Tumors

| | | | | | No. |
|---|---|---|---|---|---|
| Tumor Type | TumorType_ID | FALSE | TRUE | % TDP | Samples |
| Adrenocorti- | ACC | 12 | 5 | 29.4 | 17 |
| cal carcinoma | | | | | |
| Esophageal | ESCA | 14 | 5 | 26.3 | 19 |
| carcinoma | | | | | |
| Stomach ade- | STAD | 31 | 7 | 18.4 | 38 |
| nocarcinoma | | | | | |
| Lung squamous | LUSC | 42 | 7 | 14.3 | 49 |
| cell carcinoma | | | | | |
| Pancreatic ade- | PDAC | 340 | 33 | 8.8 | 373 |
| nocarcinoma | | | | | |

“TRUE” includes the number of tested cancers/tumors that exhibit TDP phenotype (i.e., with a positive TDP score).
“FALSE” includes the number of tested cancers/tumors that do not exhibit TDP phenotype (i.e., with a negative TDP score).
“% TDP” represents the percentage of cancers/tumors for each tumor type that has the TDP phenotype, or with a positive TDP score.

We observed that these additional TDP tumors, similar to the TNBC, ovarian carcinoma, endometrial carcinoma and hepatocellular carcinoma, all of these additional tumors exhibiting a positive TDP score. We believe that these additional TDP tumors have high susceptibility against platinum-based therapeutic agents, particularly when such platinum-based therapeutic agents are used as front-line/primary treatment. The platinum-based therapeutic agents include, e.g., cisplatin and/or carboplatin. The platinum-based therapeutic agents may therefore be used as front-line chemotherapeutic agents in these patient.

### Materials and Methods

### WGS Datasets and TDP Classification

A catalogue of somatic structural variation data was compiled from a number of WGS studies, comprising a total of 277 tumor samples (data not shown). The available structural variation information was manually curated (relative orientation and mapping coordinates of the discordant mate-pair or paired-end read clusters) from every individual study to classify each reported somatic event into one of the four basic rearrangements: deletion, tandem duplication, inversion or inter-chromosomal translocation. For studies that reported structural variation coordinates relative to the hg18 reference human genome, a lift over to hg19 was performed using the Galaxy Lift-Over tool (https://usegalaxy.org).

The procedure to calculate the TDP score is described above. A visualization of the TDP score distribution density plot across all samples suggested a trimodal distribution (FIG. 6A). The normalmixEM function of the mixtools package in R was used to fit different numbers of mixture components (up to 5) to the TDP score value distribution (SO), using default estimates as the starting values for the iterative procedure. The resulting mixture model estimates were compared using the Bayesian information criterion, and it was found that a trimodal distribution corresponded to the optimal fit.

### TCGA Genomic Datasets

Affymetrix SNP 6.0 copy number variation (CNV) datasets for primary tumor tissues were downloaded from the TCGA Data Portal in the form of level 3 CNV data type (CNV segments). Primary tumor samples from the TCGA breast invasive carcinoma dataset were classified as triple negative breast (TNB) or non-triple negative breast (NTNB) cancers, according to TCGA clinical annotations (https://tcga-data.nci.nih.gov/tcga/).

TCGA somatic mutation data for the TNB, NTNB, OV and UCEC datasets were downloaded from the UCSC Cancer Genomic Browser (https://genome-cancer.ucsc.edu), as gene-based somatic mutation calls generated by the TCGA PANCANCER Analysis Working Group. For each sample, any gene affected by at least one non-silent somatic mutation (nonsense, missense, short insertion/deletion, splice site mutation, stop codon read-through) was considered somatically mutated.

RNAseq gene expression data for the TNB, NTNB, OV and UCEC datasets were downloaded from the TCGA Data Portal in the form of level 3 RSEM raw expression estimates, generated using the TCGA RNA Sequencing Version 2 analysis pipeline. Raw gene read counts were then scale-normalized using the trimmed mean of M-values normalization method before being converted into log-counts per million with associated precision weights using the voom transformation included in the limma package in R.

### Detection of TD-Like Features Based On Copy Number Profiling

Based on the assumption that an isolated tandem duplication within any given genomic locus will result in a chromosomal segment with uniform, increased copy number compared to its two adjacent genomic regions, SNP-array genomic data was scanned for CNV profiles indicative of TD-like features, i.e., copy number segments with length ranging from 1 Kb to 2 Mb, characterized by a copy number increase of one or more units and flanked by segments of equal copy number (FIG. 7A and Ng et al.). The identified TD-like features were then used to compute TDP scores following the same metric and threshold applied for WGS data (as described in the Results section).

### Analysis of Differential Gene Expression

To identify differentially expressed genes between any two given groups of samples, the RNAseq expression dataset was first filtered and only genes whose expression value was >1 in at least n−1 samples (with n=number of samples in the smallest sample group, i.e., TDP or non-TDP) were retained for further analysis. Sample group comparisons were carried out using the moderated t statistic of the limma package in R. An FDR-adjusted P-value threshold of 0.05 was used to identify differentially expressed genes.

### Gene Ontology Enrichment Analysis

Gene enrichment analyses for Gene Ontology (GO) terms were carried out using the topGO package in R. Briefly, predefined lists of interesting genes were tested for their enrichment in GO terms against the all-gene background using the Fisher's exact test as the test statistics and the Eliminating Genes (dim) algorithm as the method for GO graph structure. GO terms with less than 10 annotated genes were removed from the analysis.

### Cell Culture and IC₅₀ Determination

All of the cell lines were purchased from the American Type Culture Collection (ATCC). They were authenticated by Short Tandem Repeat (STR) DNA profiling and regularly tested for Mycoplasma contamination using the MycoAlert PLUS Mycoplasma Detection Kit (Lonza). MB436, HCC38, HCC1187, HCC1395, MDA-MB231, HCC1937, HCC1599, HCC1143, HCC70, DU4475, MDA-MB157, HCC1806 were maintained in RPMI with 10% fetal bovine serum (FBS). BT549 was maintained in DMEM with 10% v/v FBS, and Hs578T in DMEM with 10% FBS and 0.01 mg/ml bovine insulin. IC₅₀ value determinations were obtained by plating target cells in 96 well plates at a density of 1-5×10³ cells per well. After twenty-four hours, cisplatin (Santa Cruz Biotechnology, Inc.) or carboplatin (Selleck Chemicals) were added to the culture medium in half-log serial dilutions in the range of 3.3 nM to 100 μM, in triplicate wells. Cells were incubated for 72 hours before assessing cell viability using the WST-8 assay (Dojindo Molecular Technologies, Inc.). Absorbance values were normalized to control wells (medium only) and IC₅₀ values were calculated using the IC₅₀R package. Two independent replicate experiments were carried out for each cell line and each treatment and the average IC₅₀ value from the two experiments was used for the analysis.

### Whole-Genome Sequencing of TNBC Cell Lines

Cell line genomic DNA was isolated from ˜1×10⁶ cells using a DNeasy kit (QIAGEN) and fragmented using Covaris E220 (Covaris, Woburn, Mass., USA) to a range of sizes centered on 500 bp. Paired-end DNA libraries were constructed using NEBNext DNA Library Prep Master Mix set for Illumina (New England BioLabs, Ipswich, Mass., USA) including a bead based size selection to select for inserts with an average size of 500 bps and 10 cycles of PCR. The resulting libraries were quantified by QPCR and pooled in groups of two before being sequenced on one lane of an Illumina HiSeq 2500 platform. Fastq files were paired and run through the NGSQCToolkit (v2.3, IlluQC_PRLL.pl) with a quality control cutoff of 30, before alignment to the human reference genome (NCBI Build 37 from the 1000 genomes project) using bwa (v0.7.4) and default parameters (bwa mem). The HYDRA-MULTI algorithm was used to predict structural variation events. All datasets were analyzed at the same time and structural variation events were filtered as described in. Only structural variations exclusive to individual datasets were considered for further analysis. WGS data are freely available from the Sequence Read Archive (SRA) database (http://www.ncbi.nlm.nih.gov/sra) under project ID SRP057179.

### TD-Inside and TD-Boundary Genes

A catalog of genes mapping to regions spanned by a TD (TD-inside genes, i.e., genes which are completely embedded inside a TD) in breast cancer was generated by intersecting gene bodies' coordinates (defined according to the TCGA General Annotation Files library) with TDs' coordinates for the 23 TDP breast cancer samples analyzed, and requiring TDs to overlap 100% of each gene feature. The 5% largest TDs were removed from the analysis, as they are more likely to generate gene count biases, which resulted in a total number of 3,475 TDs, with a maximum span size of 4.1 Mb. Conversely, gene features that are only partially spanned by any given TD (i.e., genes whose bodies are interrupted by at least one TD breakpoint) were labeled as TD-boundary genes.

To identify genes found inside or at the boundary of TDs at a statistically significant frequency (i.e., frequently TD-affected genes), observed gene counts were compared to expected values as estimated through 1,000 random gene samplings. For each sampling, the number of TD-inside and TD-boundary genes were computed, and the value corresponding to the median gene count+2 standard deviations was stored to build empirical distributions of expected TD-inside and TD-boundary gene counts. Frequently TD-affected genes were then identified by setting a gene count threshold equal to the round-up integer of the maximum value obtained in the empirical distributions. According to this calculation, any gene characterized by a count equal or higher than 2 was considered significantly frequent.

In a similar way, lists of genes frequently affected by TD-like features were computed using the TDs' coordinates corresponding to a total of 418 TDP tumors analyzed using SNP-array data (TNB, NTNB, OV and UCEC datasets). Based on the gene count empirical distribution obtained by generating 1,000 random gene samplings, significance thresholds was set at 5 and 11 for genes frequently found at the TD boundaries and inside TDs, respectively (FIG. 9B and data not shown).

Cancer Gene Lists A list of 1,035 known tumor suppressor genes (TSGs) was generated as the union of: (i) known recessive tumor suppressor genes according to the Cancer Gene Census; (ii) homozygously inactivated genes observed by whole-genome sequencing in the COSMIC database; (iii) genes tagged by “Entrez Query: Tumour Suppressor” in the CancerGenes database (genes which also matched the “Entrez Query: Oncogene” search were considered ambiguous and manually reassigned to the correct gene list in case of clear literature evidence, or excluded from both lists, in case of uncertainty); (iv) Human protein coding TSGs as described in the TSGene database. Of these, 1,020 genes matched gene symbols reported in the TCGA expression dataset and were used for enrichment analysis. A list of 962 known oncogenes was generated as the union of: (i) Gene tagged by “Entrez Query: Oncogene” in the CancerGenes database (genes which also matched the “Entrez Query: Tumour Suppressor” search were considered ambiguous and manually reassigned to the correct gene list in case of clear literature evidence, or excluded from both lists, in case of uncertainty); (ii) genes amplified and overexpressed in cancer; (iii) essential genes. Of these, 921 genes matched gene symbols reported in the TCGA expression data set and were used for enrichment analysis.

STOP and GO genes were identified as genes which negatively and positively regulate cell proliferation, respectively, through a genome-wide shRNA screening by Solimini et al. Of the 3,596 STOP and 1,127 GO genes identified in the study, 3,377 and 1,039 matched gene symbols reported in the TCGA expression data set respectively and were used for enrichment analysis.

Genes associated with breast cancer patients' prognosis data (good and poor prognosis genes) were identified as previously described.

### Pol2 and Histone Modification Chip-Seq Data Retrieval and Enrichment Analysis

Peaks corresponding to a total of 43 Pol2 ChIP-seq experiments across a variety of cell lines were downloaded from the ENCODE January 2011 data freeze. Histone modification peaks relative to the HMEC cell line were downloaded from ENCODE, under accession number GSE29611. Histone modification data relative to the vHMEC (variant HMEC) cell line were obtained from the NIH Roadmap Epigenomics Project (GSE16368). Peaks were called using the MACS2.09 software with the following settings “macs2 callpeak—nomodel and—shiftsize 100—broad—keep-dup=1”, and using matching input ChIP-seq datasets.

The enrichment of Pol2 binding and histone modification marks in the vicinity of TD breakpoints was calculated as described elsewhere. Briefly, for each breast cancer TD breakpoint we defined a symmetrical genomic window extending 200 Kb upstream and downstream of the breakpoint coordinate. The fraction of Pol2 binding regions or histone modification peaks falling within the collection of TD breakpoint windows were then computed. Finally, odds ratios and z-scores of the enrichment/depletion of Pol2 protein binding or histone modification marks within the defined TD breakpoint windows were calculated.

### Patient-Derived Xenografts

TNBC patient-derived xenograft models were established at The Jackson Laboratory campus in Sacramento (JAX-West) and tested for cisplatin sensitivity as previously published. All animal procedures were performed under IACUC protocol #12027. Briefly, patient tumor material acquired from biopsy or surgical resection was implanted subcutaneously into the flank of NOD-scid IL2r gamma-chain null female mice (8-10 week-old). Models were considered “established” when log-phase growth in a second passage was evident. Individual tumor-bearing mice were randomized into treatments cohorts of at least six animals each on an accrual basis when tumors reached a volume of 150 mm³ (day 0), at which point each tumor model was assessed for its response to cisplatin treatment, administrated at a dose of 2 mg/kg body weight and following a three-week regimen consisting of one IV injection per week. Changes in tumor volumes were measured twice a week for four full weeks from the beginning of the treatment or until tumor volumes reached the 1500 mm³ endpoint. Treatment outcome was evaluated in terms of total response (percentage of tumor shrinkage/growth at day 20 relative to day 0). The seven TNBC PDX models that were available as part of The Jackson Laboratory inventory of TNBC PDX Live™ tumor-bearing mice were analyzed. In combination with a high number of replicates per model (6-10 animals per treatment arm), enough power to observe the statistically significant effect of the TDP configuration on cisplatin response was obtained.

A fragment of the original engrafted tumors was used for DNA and RNA isolation. Nextera mate-pair genomic libraries were generated and sequenced on a HiSeq 2500 Illumina platform, as described elsewhere. Sequenced reads were analyzed through Xenome against a combined human Hg19 and mouse Mm10 reference to identify and remove any mouse contaminant read pairs. Structural variations were then predicted using a custom structural variation pipeline that combines the HYDRA-MULTI and DELLY algorithms. Structural variation data obtained from the peripheral blood lymphocyte DNA of four independent individuals were used to remove germline variants.

RNAseq libraries were generated following the Illumina TruSeq paired-end library preparation protocol and were sequenced on a HiSeq 2500 Illumina platform. Following the filtering of mouse reads using Xenome, human-specific paired-end reads were aligned to the hg19/GRCh37-based “UCSC gene” reference transcriptome using Bowtie2 and RSEM was used to estimate the abundance of each individual gene. Upper quartile normalization was performed within each tumor sample after discarding genes with 0 counts. Finally, gene expression levels were adjusted using a percentile rank transformation.

### OncoPrints

Oncoprints were generated using the OncoPrinter tool from the cBioPortal website.

### REFERENCES

1. Hanahan D & Weinberg R A (2011) Hallmarks of cancer: the next generation. Cell 144(5):646-674.
2. Yates L R & Campbell P J (2012) Evolution of the cancer genome. Nature reviews. Genetics 13(11):795-806.
3. Stratton M R, Campbell P J, & Futreal P A (2009) The cancer genome. Nature 458(7239):719-724.
4. Baca S C, et al. (2013) Punctuated evolution of prostate cancer genomes. Cell 153(3):666-677.
5. Stephens P J, et al. (2011) Massive genomic rearrangement acquired in a single catastrophic event during cancer development. Cell 144(1):27-40.
6. Zhang C Z, Leibowitz M L, & Pellman D (2013) Chromothripsis and beyond: rapid genome evolution from complex chromosomal rearrangements. Genes & development 27(23):2513-2530.
7. Zhang F, Carvalho C M, & Lupski J R (2009) Complex human chromosomal and genomic rearrangements. Trends in genetics: TIG 25(7):298-307.
8. Gisselsson D, et al. (2000) Chromosomal breakage-fusion-bridge events cause genetic intratumor heterogeneity. Proceedings of the National Academy of Sciences of the United States of America 97(10):5357-5362.
9. Inaki K, et al. (2014) Systems consequences of amplicon formation in human breast cancer. Genome research 24(10):1559-1571.
10. Stephens P J, et al. (2009) Complex landscapes of somatic rearrangement in human breast cancer genomes. Nature 462(7276):1005-1010.
11. Ng C K, et al. (2012) The role of tandem duplicator phenotype in tumour evolution in high-grade serous ovarian cancer. The Journal of pathology 226(5):703-712.
12. Hillmer A M, et al. (2011) Comprehensive long-span paired-end-tag mapping reveals characteristic patterns of structural variations in epithelial cancer genomes. Genome research 21(5):665-675.
13. Natrajan R, et al. (2012) A whole-genome massively parallel sequencing analysis of BRCA1 mutant oestrogen receptor-negative and -positive breast cancers. The Journal of pathology 227(1):29-41.
14. Nik-Zainal S, et al. (2012) Mutational processes molding the genomes of 21 breast cancers. Cell 149(5):979-993.
15. McBride D J, et al. (2012) Tandem duplication of chromosomal segments is common in ovarian and breast cancer genomes. The Journal of pathology 227(4):446-455.
16. Imielinski M, et al. (2012) Mapping the Hallmarks of Lung Adenocarcinoma with Massively Parallel Sequencing. Cell 150(6):1107-1120.
17. Yang L, et al. (2013) Diverse mechanisms of somatic structural variations in human cancer genomes. Cell 153(4):919-929.
18. Grzeda K R, et al. (2014) Functional chromatin features are associated with structural mutations in cancer. BMC genomics 15:1013.
19. Cancer Genome Atlas Research N, et al. (2013) Integrated genomic characterization of endometrial carcinoma. Nature 497(7447):67-73.
20. Hastings P J, Lupski J R, Rosenberg S M, & Ira G (2009) Mechanisms of change in gene copy number. Nature reviews. Genetics 10(8):551-564.
21. De S & Michor F (2011) DNA replication timing and long-range DNA interactions predict mutational landscapes of cancer genomes. Nature biotechnology 29(12):1103-1108.
22. Sima J & Gilbert D M (2014) Complex correlations: replication timing and mutational landscapes during cancer and genome evolution. Current opinion in genetics & development 25:93-100.
23. Chen C L, et al. (2010) Impact of replication timing on non-CpG and CpG substitution rates in mammalian genomes. Genome research 20(4):447-457.
24. Cancer Genome Atlas N (2012) Comprehensive molecular portraits of human breast tumours. Nature 490(7418):61-70.
25. Cancer Genome Atlas Research N (2011) Integrated genomic analyses of ovarian carcinoma. Nature 474(7353):609-615.
26. Caillat C & Perrakis A (2012) Cdtl and geminin in DNA replication initiation. Sub-cellular biochemistry 62:71-87.
27. Powell S K, et al. (2015) Dynamic loading and redistribution of the Mcm2-7 helicase complex through the cell cycle. The EMBO journal 34(4):531-543.
28. Yang W, et al. (2013) Genomics of Drug Sensitivity in Cancer (GDSC): a resource for therapeutic biomarker discovery in cancer cells. Nucleic acids research 41(Database issue):D955-961.
29. von Minckwitz G & Martin M (2012) Neoadjuvant treatments for triple-negative breast cancer (TNBC). Annals of oncology: official journal of the European Society for Medical Oncology/ESMO 23 Suppl 6:vi35-39.
30. Davis S L, Eckhardt S G, Tentler J J, & Diamond J R (2014) Triple-negative breast cancer: bridging the gap from cancer genomics to predictive biomarkers. Therapeutic advances in medical oncology 6(3):88-100.
31. Stefansson O A, Villanueva A, Vidal A, Marti L, & Esteller M (2012) BRCA1 epigenetic inactivation predicts sensitivity to platinum-based chemotherapy in breast and ovarian cancer. Epigenetics: official journal of the DNA Methylation Society 7(11):1225-1229.
32. Silver D P, et al. (2010) Efficacy of neoadjuvant Cisplatin in triple-negative breast cancer. Journal of clinical oncology: official journal of the American Society of Clinical Oncology 28(7):1145-1153.
33. Fong P C, et al. (2009) Inhibition of poly(ADP-ribose) polymerase in tumors from BRCA mutation carriers. The New England journal of medicine 361(2):123-134.
34. Farmer H, et al. (2005) Targeting the DNA repair defect in BRCA mutant cells as a therapeutic strategy. Nature 434(7035):917-921.
35. Liu P, et al. (2011) Chromosome catastrophes involve replication mechanisms generating complex genomic rearrangements. Cell 146(6):889-903.
36. Korbel J O & Campbell P J (2013) Criteria for inference of chromothripsis in cancer genomes. Cell 152(6):1226-1236.
37. Green B M, Finn K J, & Li J J (2010) Loss of DNA replication control is a potent inducer of gene amplification. Science 329(5994):943-946.
38. Finn K J & Li J J (2013) Single-stranded annealing induced by re-initiation of replication origins provides a novel and efficient mechanism for generating copy number expansion via non-allelic homologous recombination. PLoS genetics 9(1):e1003192.
39. Costantino L, et al. (2014) Break-induced replication repair of damaged forks induces genomic duplications in human cells. Science 343(6166):88-91.
40. Medvedev P, Stanciu M, & Brudno M (2009) Computational methods for discovering structural variation with next-generation sequencing. Nature methods 6(11 Suppl):S13-20.
41. Benaglia T, Chauveau D, Hunter D R, & Young D S (2009) mixtools: An R Package for Analyzing Finite Mixture Models. J Stat Softw 32(6):1-29.
42. Smyth G K (2004) Linear models and empirical bayes methods for assessing differential expression in microarray experiments. Statistical applications in genetics and molecular biology 3:Article3.
43. Alexa A, Rahnenfuhrer J, & Lengauer T (2006) Improved scoring of functional groups from gene expression data by decorrelating G O graph structure. Bioinformatics 22(13):1600-1607.
44. Frommolt P & Thomas R K (2008) Standardized high-throughput evaluation of cell-based compound screens. BMC bioinformatics 9:475.
45. Lindberg M R, Hall I M, & Quinlan A R (2014) Population-based structural variation discovery with Hydra-Multi. Bioinformatics.
46. Malhotra A, et al. (2013) Breakpoint profiling of 64 cancer genomes reveals numerous complex rearrangements spawned by homology-independent mechanisms. Genome research 23(5):762-776.
47. Futreal P A, et al. (2004) A census of human cancer genes. Nature reviews. Cancer 4(3):177-183.
48. Forbes S A, et al. (2008) The Catalogue of Somatic Mutations in Cancer (COSMIC). Current protocols in human genetics/editorial board, Jonathan L. Haines . . . [et al.] Chapter 10:Unit 10 11.
49. Higgins M E, Claremont M, Major J E, Sander C, & Lash A E (2007) CancerGenes: a gene selection resource for cancer genome projects. Nucleic acids research 35(Database issue):D721-726.
50. Zhao M, Sun J, & Zhao Z (2013) TSGene: a web resource for tumor suppressor genes. Nucleic acids research 41(Database issue):D970-976.
51. Santarius T, Shipley J, Brewer D, Stratton M R, & Cooper C S (2010) A census of amplified and overexpressed human cancer genes. Nature reviews. Cancer 10(1):59-64.
52. Solimini N L, et al. (2012) Recurrent hemizygous deletions in cancers may optimize proliferative potential. Science 337(6090):104-109.
53. Consortium E P (2012) An integrated encyclopedia of DNA elements in the human genome. Nature 489(7414):57-74.
54. Bernstein B E, et al. (2010) The NIH Roadmap Epigenomics Mapping Consortium. Nature biotechnology 28(10):1045-1048.
55. Feng J, Liu T, Qin B, Zhang Y, & Liu X S (2012) Identifying ChIP-seq enrichment using MACS. Nat Protoc 7(9):1728-1740.
56. Zhang Y, et al. (2008) Model-based analysis of ChIP-Seq (MACS). Genome Biol 9(9):R137.
57. Shultz L D, et al. (2014) Human cancer growth and therapy in immunodeficient mouse models. Cold Spring Harbor protocols 2014(7):694-708.
58. Conway T, et al. (2012) Xenome—a tool for classifying reads from xenograft samples. Bioinformatics 28(12):i172-178.
59. Lindberg M R, Hall I M, & Quinlan A R (2014) Population-based structural variation discovery with Hydra-Multi. Bioinformatics.
60. Malhotra A, et al. (2013) Breakpoint profiling of 64 cancer genomes reveals numerous complex rearrangements spawned by homology-independent mechanisms. Genome research 23(5):762-776.
61. Rausch T, et al. (2012) DELLY: structural variant discovery by integrated paired-end and split-read analysis. Bioinformatics 28(18):i333-i339. 62. Li B & Dewey C N (2011) RSEM: accurate transcript quantification from RNA-Seq data with or without a reference genome. BMC bioinformatics 12:323.
63. Cerami E, et al. (2012) The cBio cancer genomics portal: an open platform for exploring multidimensional cancer genomics data. Cancer discovery 2(5):401-404.
64. Gao J, et al. (2013) Integrative analysis of complex cancer genomics and clinical profiles using the cBioPortal. Science signaling 6(269):p 11.

All references cited herein are incorporate by reference.

What is claimed is:

1. A method of treating a cancer patient suffering from triple negative breast cancer, ovarian cancer, hepatocellular carcinoma, or endometrial carcinoma, said method comprising:
(a) obtaining a tumor sample from the cancer patient;
(b) determining tandem duplications of said tumor sample;
(c) determining a TDP Score using Formula (I):
$$TDP Score = TDP Raw Score + k = - \frac{{Σ}_{i} \vert {Obs}_{i} - {Exp}_{i} \vert}{TD} + k \qquad ( I )$$
wherein:
TD is total number of tandem duplications,
Obsᵢ is observed number of tandem duplications for each chromosome i,
Expᵢ is expected number of tandem duplications for each chromosome i, and,
k is 0.71; and
(d) administering a therapeutically effective amount of a platinum-based therapeutic agent to the cancer patient, when the TDP Score is >0.

2. The method of claim 1, wherein said determining step in step (b) is performed using whole-genome sequencing (WGS), or SNP-array analysis.

3. The method of claim 2, wherein said whole-genome sequencing (WGS) is performed using Next Generation Sequencing (NGS).

4. The method of claim 3, wherein said Next Generation Sequencing is performed using Illumia HisSeq 2500 platform.

5. The method of any one of claims 1-4, wherein said cancer patient suffers from a triple negative breast cancer.

6. The method of any one of claims 1-4, wherein said cancer patient suffers from an ovarian cancer.

7. The method of any one of claims 1-4, wherein said cancer patient suffers from a hepatocellular carcinoma.

8. The method of any one of claims 1-4, wherein said cancer patient suffers from an endometrial carcinoma.

9. The method of any one of claims 1-8, wherein the platinum-based therapeutic agent comprises cisplatin, carboplatin, oxaliplatin, nedaplatin, heptaplatin, lobaplatin, satraplatin, picoplatin, triplatin tetranitrate, phenanthriplatin, or a combination thereof.

10. The method any one of claims 1-9, wherein the platinum-based therapeutic agent comprises cisplatin or carboplatin.

11. A method of identifying and selecting a cancer patient suffering from triple negative breast cancer, ovarian cancer, hepatocellular carcinoma or endometrial carcinoma as a candidate suitable for a platinum-based therapy, said method comprising:
(a) obtaining a tumor sample from the cancer patient;
(b) determining tandem duplications of said tumor sample;
(c) determining a TDP Score using Formula (I):
$$TDP Score = TDP Raw Score + k = - \frac{{Σ}_{i} \vert {Obs}_{i} - {Exp}_{i} \vert}{TD} + k \qquad ( I )$$
wherein:
TD is total number of tandem duplications,
Obsᵢ is observed number of tandem duplications for each chromosome i,
Expᵢ is expected number of tandem duplications for each chromosome i, and,
k is 0.71; and,
(d) identifying and selecting the cancer patient as a candidate for the treatment of a platinum-based therapeutic agent, when said TDP Score is positive.

12. The method of claim 11, further comprising administering a therapeutically effective amount of the platinum-based therapy to said cancer patient.

13. The method of claim 11, wherein said determining step in step (b) is performed using whole-genome sequencing (WGS), or SNP-array analysis.

14. The method of claim 13, wherein said whole-genome sequencing (WGS) is performed using Next Generation Sequencing (NGS).

15. The method of claim 14, wherein said Next Generation Sequencing is performed using Illumia HisSeq 2500 platform.

16. The method of any one of claims 11-15, wherein said cancer patient suffers from a triple negative breast cancer.

17. The method of any one of claims 11-15, wherein said cancer patient suffers from a ovarian cancer.

18. The method of any one of claims 11-15, wherein said cancer patient suffers from a hepatocellular carcinoma.

19. The method of any one of claims 11-15, wherein said cancer patient suffers from an endometrial carcinoma.

20. The method of any one of claims 11-19, wherein said platinum-based therapeutic agent comprises cisplatin, carboplatin, oxaliplatin, nedaplatin, heptaplatin, lobaplatin, satraplatin, picoplatin, triplatin tetranitrate, phenanthriplatin, or a combination thereof.

21. The method any one of claims 11-20, wherein said platinum-based therapeutic agent comprises cisplatin or carboplatin.
